(19) United States
(12) Patent Application Publication
(10) Pub. No.: US 2009/0325311 A1
(43) Pub. Date: Dec. 31, 2009

(54) Measurement methods

(75) Inventors: Jean-Michel Lecerf, Cambridge, MA (US); William J. Riordan, Mansfield, MA (US); Frank Y. Hsieh, Lexington, MA (US); Edward Joseph Takach, Arlington, MA (US); Qing Zhu, W. Roxbury, MA (US); Guangyu Qian, Newton, MA (US); Mike Annunziato, Mansfield, MA (US); Kannappan Veeraragavan, Westborough, MA (US)

Correspondence Address: LANDO & ANASTASI, LLP, ONE MAIN STREET, SUITE 1100, CAMBRIDGE, MA, 02142, US

(73) Assignee: MILLENNIUM PHARMACEUTICALS, INC.

(21) Appl. No.: 11/725,252

(22) Filed: Mar. 19, 2007

Related U.S. Application Data
(63) Continuation of application No. 11/130,543, filed on May 16, 2005, now abandoned.
(60) Provisional application No. 60/571,382, filed on May 14, 2004.

(51) Int. Cl. G01N 33/566

(52) U.S. Cl. 436/501

(57) ABSTRACT

The invention relates to methods for measuring unconjugated molecules, e.g. antibodies, in a mixture that also includes conjugated molecules, e.g. antibodies.

### CLAIM OF PRIORITY

This application claims the benefit of U.S. application Ser. No. 11/130,543, filed on May 16, 2005 which claims the benefit of U.S. Provisional Application Ser. No. 60/571,382, filed on May 14, 2004, the entire contents of which are hereby incorporated by reference.

### TECHNICAL FIELD

This invention relates to methods for evaluating impurities, or chemical, biochemical, or bioconjugation changes (e.g., resulting from degradation or metabolism) in a sample which includes a conjugated molecule.

### BACKGROUND

Numerous therapeutic conjugates, e.g., conjugated antibodies, are in use or being developed. These compounds typically include an antigen-specific binding agent such as an antibody or antigen-binding fragment thereof, and a cytotoxic or detectable label conjugate. The conjugate can be radio-labeled. Controlling the quality of these biotherapeutic molecules is crucial for patient safety, proper dosing, and batch-to-batch reproducibility of therapeutic efficacy. Impurities resulting from the manufacturing process, including unconjugated and semi-conjugated intermediates, may affect the therapeutic performance of the compounds.

### SUMMARY

The invention is based, in part, on the development of methods for evaluating a sample of a conjugated molecule having a first member and a second member. The evaluation can include, e.g., quantitatively measuring an impurity, e.g., an unreacted intermediate; the product of breakdown of a conjugated molecule which separates the first member from the second member and results in a first member molecule (in-vitro or in-vivo) which is not conjugated to the second member; the product of partial breakdown of a conjugated molecule resulting in changes of conjugation ratios; or a conjugate-related impurity, e.g., as described herein. In one aspect, the invention provides for detecting unconjugated first members, e.g., antibody molecules, e.g., unconjugated anti-PSMA antibodies, in a sample. The method includes:

(1) Contacting the sample with a binding agent (e.g., an antibody or antigen-binding fragment thereof, or other ligand) that is specific for a conjugate (e.g., a toxin, radio-labeled element, chromogen, enzyme, fluorochrome, hapten, or biotin), conjugated to the first member, e.g., where the first member is an antibody, to deplete conjugated molecule, e.g., a conjugated molecule where the first member is an antibody molecule, from the sample and form a depleted sample. A preferred conjugate is a maytansinoid, e.g., DM1, DM4 or a maytansinol. Another preferred conjugate can be a linker, e.g., DOTA or SPP. The conjugate-specific binding agent can be immobilized on a substrate, typically a substrate which is insoluble in the samples and buffers used, which can be separated from the sample, e.g., it can be attached to beads or to the surface of a container, e.g., the surface of a microtiter plate. The sample is contacted with the conjugate-binding agent under conditions which allow depletion of the conjugated molecule from the sample. One or more rounds of depletion are performed;

(2) Contacting at least a portion of the depleted sample with a second binding agent which is specific for the first member portion, e.g., an antibody portion, of the conjugated molecules. The second, first member-specific, e.g., antibody-specific, binding agent can be immobilized on a substrate, typically a substrate which is insoluble in the samples and buffers used, which can be separated from the depleted sample, e.g., it can be attached to beads or to the surface of a container, e.g., the surface of a microtiter plate. The depleted sample is contacted with the second binding agent under conditions which allow binding to the first member molecules, e.g., antibody, in the depleted sample. (Optionally, as a control to monitor the extent of depletion, at least a portion of the depleted sample can be incubated with a binding agent specific for the conjugate, e.g., an antibody or antigen-binding fragment thereof that binds specifically to the entity conjugated to the antibody and which preferably is labeled); and

(3) Evaluating the presence or amount of first member, e.g., antibody, bound by the first member-specific agent, e.g., an antibody-specific binding agent. Evaluation can include contact of the subject antibody with an agent which binds to the subject antibody, e.g., an anti-idiotypic antibody, or an antibody to the constant region of the antibody being evaluated. Typically this agent is directly or indirectly labeled. The presence of unconjugated antibody is indicative of degradation. The evaluation can include comparing the fraction or amount of unconjugated first member, e.g., antibody, molecules in the depleted sample with a reference value, e.g., a value from a reference standard, e.g., a value from a standard curve of known concentration of conjugated or unconjugated antibody molecules. Unconjugated first member molecules, e.g., antibody molecules, can arise, e.g., from incomplete conjugation or from degradation.

In a preferred embodiment the first member and the second member, or conjugate, are linked by a disulfide S—S bond. The S—S bond can be formed between an S of the first member and an S of the second member, or between an S of the first or second member and an S of a linker molecule. In a preferred embodiment, the method detects first member molecules released from the conjugate by breakage of an S—S bond.

In a preferred embodiment the conjugate-specific binding agent is an antibody. In a preferred embodiment, the conjugate specific binding agent binds directly to the conjugate, and can be an antibody.

In a preferred embodiment the antibody-specific binding agent is an antibody. In a preferred embodiment the antigen-specific binding agent binds directly to the first member, e.g., an antibody, and can be an antibody.

In a preferred embodiment the sample includes conjugated and unconjugated antibody molecules and the method includes depleting substantially all of the conjugated antibody molecules from the sample and detecting antibody molecules remaining in the sample.

The information from the method can be used to evaluate a sample, e.g., to determine whether to use (or how to use, e.g., how much to use of) a sample, batch or preparation of conjugated molecule for a preselected purpose, e.g., for formulation, combination with another component, calculation of a dose, measurement or division into doses, e.g., unit doses, packaging, disposal, labeling, administration to a subject, or a decision on whether to execute or perform a second event. Typically, the evaluation can include comparing the fraction or amount of unconjugated first member, e.g., antibody molecules, in the depleted sample with a reference value, e.g., a value from a reference standard, e.g., a value from a standard curve of known concentration of conjugated or unconjugated antibody molecules. If the value for the sample meets a preselected criteria, e.g., it is less than a preselected value, it is used for the preselected purpose.

In a preferred embodiment the method is performed more than once on a sample, and thus allows for the evaluation, e.g., evaluation of the stability of a sample, over time. In such embodiments the method includes:

providing a first portion of the sample at a first point in time, and evaluating it by a method described herein, e.g., depleting, e.g., depleting substantially all of, the conjugated molecules from the first portion; detecting first member, e.g., antibody, molecules, remaining in the first portion, thereby determining a first level of unconjugated first member, e.g., antibody, molecules in the sample; and

providing a second portion of the sample at a second point in time and evaluating it by a method described herein, e.g., depleting, e.g., depleting substantially all of, the conjugated molecules from the second portion; and detecting first member, e.g., antibody, molecules remaining in the second portion, thereby determining a second level of unconjugated first member, e.g., antibody molecules;

thereby evaluating the sample, e.g., evaluating the stability of the conjugated molecules. Evaluating can include comparing one or both the amount detected at the first and second point in time with one or more reference values, e.g., comparing the amount of antibody detected at the first point in time with the amount detected at the second point in time. In a preferred embodiment an increase in the level of unconjugated first member, e.g., antibody, molecules between the first portion and the second portion correlates negatively with the stability of the conjugated molecule in the sample. In a preferred embodiment the levels remain the same and the sample is considered stable.

In some embodiments, the first time point occurs at or near a first preselected time or event, (e.g., within 1, 5, 10, 24, 48, or 96 hours of the first preselected time or event), and the second time point occurs at or near a second preselected time or event, (e.g., within 1, 5, 10, 24, 48, or 96 hours of the second preselected time or event). The first event can be production or purification and the second event can be formulation, combination with another component, calculation of a dose, measurement or division into doses, e.g., unit doses, packaging, disposal, labeling administration to a subject, or a decision on whether to perform or execute a second event. In some embodiments, the first time point is within 1, 5, 10, 24, 48, or 96 hours of purification of the sample, and the second time point is after storage of the sample for 1, 2, 3, 4, 5, 6, 7, 8, 10, or more months, or 1, 2, 3, 4, 5 or more years. In some embodiments, the first time point is after purification of the sample, and the second time point is after formulation of the drug substance.

In some embodiments, the sample includes a biological fluid, e.g., serum, blood, or urine. In other embodiments, the sample is from a batch of conjugated antibody molecules or the sample is a formulated conjugated antibody molecule product.

In some embodiments, the unconjugated antibody molecule is an anti-PSMA antibody molecule, and the conjugated antibody molecule is a conjugated anti-PSMA antibody molecule. In some embodiments, the anti-PSMA antibody molecule is selected from the group consisting of E99, J415, J533, and J591, e.g., deJ591. In other embodiments, the anti-PSMA antibody is an anti-PSMA antibody described herein.

In some embodiments, the conjugated molecule includes a cytotoxic conjugate, e.g., a compound emitting radiation, molecules of plant, fungal, or bacterial origin, or a biological protein or particle. In particularly preferred embodiments, the cytotoxic conjugate comprises a maytansinoid, e.g., DM1, DM4 or a maytansinol. In some embodiments, the conjugated molecule comprises a detectable conjugate, e.g., biotin or other detectable conjugate described herein.

In some embodiments, detection of first member, e.g., antibody, molecules remaining in the sample includes contacting the sample with a binding agent specific for the first member, e.g., antibody, molecules (e.g., the binding agent can be an antibody or functional fragment thereof, or other ligand specific for the antibody molecule), and detecting the binding agent. In some embodiments, the binding agent specific for the first member, e.g., antibody, molecules is directly or indirectly labeled.

In some embodiments, the binding agent specific for the first member, e.g., antibody, molecule is an anti-idiotypic antibody.

In some embodiments, the binding agent specific for the conjugate is an antibody or functional fragment thereof. In some embodiments, the binding agent specific for the conjugate is directly or indirectly labeled.

In some embodiments, depleting substantially all of the conjugated molecule, e.g., antibody, removes greater than about 90% of the conjugated molecule, e.g., greater than about 95%, 97%, 98%, or 99% of the conjugated molecule.

In some embodiments, the sample comprises a DM1 conjugated J591 antibody molecule, e.g., a DM1 conjugated deJ591 antibody molecule, e.g., a formulated DM1 conjugated deJ591 antibody molecule also referred to herein as a DS-DM1-deJ591 conjugated antibody molecule.

In some embodiments, the sample comprises less than about 5%, 4%, 3%, 2%, 1%, 0.8%, 0.5% or 0.2% of unconjugated molecule, e.g., antibody.

In a preferred embodiment two or more batches or production runs are evaluated, and compared. The method includes providing a first sample from a first batch of conjugated molecules and evaluating it by a method described herein; providing a second sample from a second batch of conjugated molecules and evaluating it by a method described herein; and comparing the results for the two batches.

In another aspect, the invention features, a method of quantifying unconjugated anti-PSMA antibody molecules in a sample that includes maytansinoid (e.g., maytansinol and more preferably, DM1 or DM4) conjugated anti-PSMA antibody molecules and unconjugated anti-PSMA antibody molecules. The method includes evaluating the sample using a method described herein, e.g., depleting substantially all of the maytansinoid conjugated antibody molecules from the sample using an antibody molecule that specifically binds the maytansinoid; and detecting any anti-PSMA antibody molecules remaining in the sample using an antibody molecule that binds the anti-PSMA antibody molecule. (The method can also be used to quantify unconjugated antibody molecules in a sample containing antibody-chelator, e.g., antibody-DOTA, molecules or antibody-linker, e.g., an antibody-SPP molecules.)

In another aspect, the invention features, a method of detecting unconjugated anti-PSMA antibody molecules in a sample that includes maytansinoid (e.g., maytansinol and more preferably, DM1 or DM4) conjugated anti-PSMA antibody molecules. The method includes evaluating the sample using a method described herein, e.g., depleting substantially all of the maytansinoid conjugated antibody molecules from the sample using an antibody molecule that specifically binds the maytansinoid, e.g., an antibody attached to a solid support; and detecting anti-PSMA antibody molecules remaining in the sample using an antibody molecule that binds the anti-PSMA antibody molecule.

In another aspect, the invention features a method of evaluating the stability of a sample comprising a maytansinoid-conjugated anti-PSMA antibody molecule (e.g., a maytansinol and preferably a DM1 or DM4 conjugated anti-PSMA antibody molecule). The method includes;

providing a first portion of the sample at a first point in time, and evaluating it by a method described herein, e.g., depleting, e.g., depleting substantially all of, the maytansinoid conjugated anti-PSMA antibody molecules from the first portion; detecting antibody molecules remaining in the first portion, thereby determining a first level of unconjugated antibody molecules in the sample; and

providing a second portion of the sample at a second point in time and evaluating it by a method described herein, e.g., depleting, e.g., depleting substantially all of, the maytansinoid conjugated anti-PSMA antibody molecules from the second portion; and detecting antibody molecules remaining in the second aliquot, thereby determining a second level of unconjugated antibody molecules;

thereby evaluating the sample, e.g., evaluating the stability of the conjugated antibody. Evaluating can include comparing one or both the amount detected at the first and second point in time with one or more reference values, e.g., comparing the amount of antibody detected at the first point in time with the amount detected at the second point in time. In a preferred embodiment an increase in the level of unconjugated antibody molecules between the first portion and the second portion correlates negatively with the stability of the maytansinoid conjugated anti-PSMA antibody molecule in the sample—a decrease in the amount of maytansinoid conjugated anti-PSMA antibody at the second time points is indicative of a lack of stability. In a preferred embodiment the levels remain the same and the sample is considered stable.

In some embodiments, the first time point occurs at or near a first preselected time or event (e.g., within 1, 5, 10, 24, 48, or 96 hours of the first preselected time or event), and the second time point occurs at or near a second preselected time or event (e.g., within 1, 5, 10, 24, 48, or 96 hours of the second preselected time or event). The first event can be production or purification and the second event can be formulation, combination with another component, calculation of a dose, measurement or division into doses, e.g., unit doses, packaging, disposal, labeling, administration to a subject, or a decision on whether to perform or execute a second event. In some embodiments, the first time point is within 1, 5, 10, 24, 48, or 96 hours of purification of the sample, and the second time point is after storage of the sample for 1, 2, 3, 4, 5, 6, 7, 8, 10, or more months, or 1, 2, 3, 4, 5 or more years. In some embodiments, the first time point is after purification of the sample, and the second time point is after formulation of the drug substance.

In some embodiments, the maytansinoid is DM1.

In some embodiments, the maytansinoid is DM4.

In some embodiments, the method also includes contacting the maytansinoid conjugated anti-PSMA antibody molecule-depleted sample with an antibody molecule that binds the anti-PSMA antibody molecule, wherein the antibody molecule that binds the anti-PSMA antibody molecule is attached to a solid support.

In some embodiments, the anti-PSMA antibody molecules are detected using a labeled antibody molecule that binds to the anti-PSMA antibody molecule. In some embodiments, the labeled antibody molecule is directly or indirectly labeled.

In some embodiments, the anti-PSMA antibody molecule binds the extracellular domain of PSMA. In some embodiments, the anti-PSMA antibody molecule is E99, J415, J533, or J591, e.g., deJ591.

In some embodiments, the method includes comparing the detected antibody molecules to a reference, e.g., a standard curve of known concentrations of conjugated or unconjugated antibody molecules.

In another aspect, the invention features a method for evaluating a process for the provision of a conjugated molecule, e.g., a conjugated antibody, e.g., a process for purifying, conjugating, formulating, adding a component to, a conjugated molecule. The method includes providing a sample of conjugated molecules made by the process and evaluating it by a method described herein, e.g., depleting, e.g., depleting substantially all of, the conjugated molecules from the sample; and detecting first member, e.g., antibody, molecules remaining in the sample.

In some embodiments, the method includes comparing the amount of unconjugated first member, e.g., antibody, molecules remaining in the sample to a reference value, e.g., the amount of unconjugated first member, e.g., antibody, molecules remaining in a sample purified using a different process.

In another aspect, the invention features, a method for evaluating a batch of conjugated antibody molecules. The method includes providing a sample from the batch of conjugated antibody molecules and evaluating it by a method described herein, e.g., depleting, e.g., depleting substantially all of. the conjugated antibody molecules from the sample; detecting antibody molecules remaining in the sample; and comparing the amount of antibody molecules remaining in the sample to a reference standard, e.g., comparing the amount of antibody molecules remaining in the sample to the amount of antibody molecules remaining in a sample obtained from a different batch, e.g., a reference batch, or to the average amount of antibody molecules remaining in a plurality of samples obtained from more than one batch of the conjugated antibody molecule.

In another aspect, the invention features, a method of providing or calculating a dosage of a conjugated antibody. The method includes: providing an evaluation of the amount of degradation of a conjugated antibody which has occurred or will occur in a test sample, wherein the evaluation is provided by a method described herein;

comparing the amount of degradation to a reference value, and providing or calculating a dosage based on the relationship of the sample value to the reference value. E.g., if the sample value is greater than the reference value the dosage is increased, if the sample value is less than a reference value the dosage is decreased.

In a preferred embodiment the method further includes providing a unit dose of the conjugated antibody.

In a preferred embodiment the method further includes providing administering the conjugated antibody to a patient.

Methods described herein can be used to quantitate the percentage of impurities in the manufacture of a conjugated antibody drug substance. Methods described herein are sensitive and can provide a quantitative measurement of unconjugated antibodies or conjugate-related impurities (as discussed below). Methods of the invention can detect unconjugated antibody at a level below 2% of the total antibody in the sample. Thus, the methods described herein can be used as a quantitative impurity assay to determine the amount of unconjugated antibody molecules, or conjugate-related impurities, for drug substance and drug product lot release. For example, after manufacturing a conjugated antibody molecule as a biotherapeutic drug, it is important to measure the amount of unconjugated antibody molecules present in the purified drug substance and drug product, because the presence of unconjugated antibody molecules will compete with the biotherapeutic drug for binding to therapeutic targets, resulting in reduction of efficacy.

In addition, it is important to measure the rate of deconjugation of the biotherapeutic reagent over time (e.g., the stability of the conjugated antibody molecule), e.g., before release to end users. The methods described herein can also be used to assess impurities (e.g., unconjugated antibody molecules or conjugate-related impurities) during formulation changes and during process development. The methods described herein can also be used for pharmacokinetic analysis, e.g., to monitor the rate of deconjugation of the drug, along with identification and quantitation of conjugated drug substance metabolites in the patient blood circulation and to aid in designing the best frequency of drug dosage. Finally, the methods described herein can be adapted for purification as well as detailed analysis of impurities by chromatography.

In another aspect, the invention features, a method of selecting a sample, or evaluating the suitability of a sample of antibody molecules for radiolabeling. The method includes:

providing a sample comprising antibody molecules; and

determining the identity and/or amount of a metal ion, and preferably determining the amount of two or more metal ions selected from the group consisting of Fe, Ni, Co, Cu, Zn, La, Ce, and Pb in the sample

wherein the level of metal ions is negatively correlated to suitability for radiolabeling, e.g., radiolabeling with a radiolabel described herein, e.g., Yttrium and Indium. In a preferred embodiment the method includes comparing the level of one or more metal ions with a reference value. In a preferred embodiment the level is less than 25, 50, 100, 150, or 200 ng/ml for each metal. If the sample level is below the preselected reference value the sample is selected and/or radiolabeled.

In a preferred example the reference value is 100 ng/ml of each metal.

In a preferred embodiment the method includes selecting the sample for radiolabeling and, optionally, radiolabeling the sample.

In a preferred embodiment the method comprises determining the amount of Fe, Ni, Co, Cu, Zn, La, Ce, and Pb metal ions in the sample. A combined total level of less than 0.25, 0.75, 1.0, 1.25, 1.50 or 2.0 ug/ml for all of the metals is an indicator that the sample is suitable for selection and/or radiolabeling. In a preferred embodiment a combined total level of less than 1 ug/ml for all of the metals is an indicator that the sample is suitable for selecting and/or radiolabeling.

The invention also includes preparations of conjugated molecules, e.g., conjugated antibodies, which have been evaluated, selected or made with methods described herein.

In another aspect, the invention features, a method of evaluating the level of an SH-bearing species in a sample. In preferred embodiments the SH-bearing species is a drug or toxin, e.g., a drug or toxin disclosed herein, e.g., DM1, DM4, or modified taxol with an SH moiety. In particularly preferred embodiments the method is used to detect an SH-bearing moiety which has been released in the breakdown of a conjugated molecule. Typically the breakdown separates the first member from the second member of the conjugated molecule and results in a moiety having an SH moiety. The method is particularly useful for detecting the breakdown of a conjugated molecule where a first and second member are linked by an S—S bond, and the breakdown releases a drug or toxin having an SH moiety. The method is particularly useful for detecting free or unconjugated DM1 or DM4 in a sample.

The method includes: contacting the sample with a capture/detection moiety (which includes a reactive thio group) which reacts with an SH-bearing species to derivatize the SH-bearing sample, and detecting the derivatized SH-bearing species.

In a preferred embodiment the capture/detection moiety has the following structure and properties:

R₁—S—S—R₂

wherein:

R₁ is an aromatic heterocycle, e.g., a 5, 6, 7, 8, or 9 membered ring, which preferably includes at least one N (and preferably no other heteroatoms). It can be substituted or unsubstituted. It preferably has a molecular weight of less than 200, and more preferably less than 100. R₁ must be such that it results in a reactive S, e.g., by the presence of an electron withdrawing N in the heterocycle. Preferred R₁ groups are unsubstituted or substituted 5, 6, or 7 member rings having 1 or 2 N atoms (and preferably no other heteroatoms). Pyridyl is a preferred R₁; and

R₂ is an aromatic heterocycle, e.g., a 5, 6, 7, 8, or 9 membered ring, which preferably includes at least one N (and preferably no other heteroatoms). It can be substituted or unsubstituted. It preferably has a molecular weight of less than 200, and more preferably less than 100. R₂ must be such that it results in a reactive S, e.g., by the presence of an electron withdrawing N in the heterocycle. Preferred R₂ groups are unsubstituted or substituted 5, 6, or 7 member rings having 1 or 2 N atoms (and preferably no other heteroatoms). Pyridyl is a preferred R₂.

In a preferred embodiment R₁=R₂.

In a preferred embodiment R₁—S—S—R₂ is a pyridine disulfide, e.g., 4,4′-pyridine disulfide or 2,2′-pyridine disulfide.

In a preferred embodiment the capture/detection moiety reacts with the SH of DM1 or DM4 to, e.g., form an S—S bond between the capture/detection moiety and the DM1 or DM4 and can be, e.g., a pyridine disulfide, e.g., 4,4′-pyridine disulfide or 2,2′-pyridine disulfide.

The capture/detection moiety should be one which can be detected with a mass spectrometer equipped with an electrospray or chemical ionization source, or specifically with LC/MS/MS. In a preferred embodiment a polar moiety, e.g., pyridyl disulfide, is used to derivatize the free or unconjugated species, e.g., DM1 or DM4. The free or unconjugated species, e.g., DM1 or DM4, can be in biological samples such as serum, plasma or urine.

In a preferred embodiment free species, e.g., DM1 or DM4, can be detected at a concentration lower than its IC₅₀ towards cells.

The method can include: contacting the sample with a capture/detection moiety which reacts with the free or unconjugated DM1 or DM4 to form derivatized DM1 or DM4 (which is preferably more stable than free or unconjugated DM1 or DM4), and detecting the derivatized DM1 or DM4.

In a preferred embodiment the capture/detection moiety reacts with the SH of DM1 or DM4 to, e.g., form an S—S bond between the capture/detection moiety and the DM1 or DM4. The capture/detection moiety should be one which can be detected with MS. In a preferred embodiment a polar moiety, e.g., pyridyl disulfide, is used to derivatize the free or unconjugated DM1 or DM4. The free or unconjugated DM1 or DM4 can be in a biological sample such as serum, plasma or urine.

The method allows for an effective means of capturing free drug or toxin, e.g., DM1 or DM4, in a biological sample, e.g., to allow determination of its concentration at one or more time points, e.g., over a time-course after dosing.

In another aspect, the invention features analyzing a sample containing a mixture of antibody molecules (or first members as defined elsewhere herein) conjugated to a second moiety, e.g., a toxin (e.g., DM1 or DM4) or a chelator (e.g., DOTA). Conjugation to the antibody can result in a heterogeneous mixture, representing various levels of conjugation. The number of DM1 or DM4 molecules conjugated to antibody or the number of DOTA molecules conjugated to antibody can be determined. In a preferred embodiment the method uses matrix-assisted laser-desorption ionization (MALDI)-time of flight (TOF) mass spectrometry (MS) to resolve and identify the different masses of antibody representing various levels of DM1, DM4 or DOTA conjugation. The use of MALDI-TOF MS provides the advantage of using an uncomplicated and rapid bimolecular measurement, to characterize both DOTA and DM1 distribution ratios to deJ591.

In a preferred embodiment, distribution ratios for conjugation of either DOTA or DM1 to antibody are determined by selecting a peak, e.g., the doubly-protonated (2+ charge state) mass spectral peak, for the conjugated antibody, and analyzing it by Gaussian deconvolution and peak fitting. In a preferred embodiment the individual levels of conjugation are quantified using the response factor for a known concentration of unconjugated antibody to create a calibration curve and using this to quantify different levels of DM1-J591 or DOTA-J591 conjugates for each identified and measured isoform-of conjugated antibody. In a preferred embodiment this analysis can be performed for samples representing unreacted material, to monitor quality control over time, or resulting from in vitro or in vivo reactions.

In a preferred embodiment the method detects at least two, and preferably at least 3, 4, 5, 6, or 7 levels of conjugation. In a preferred embodiment an average level of conjugation is determined. In a preferred embodiment the individual levels of conjugation are quantified using the response factor for a known concentration of unconjugated antibody to create a calibration curve and using this to quantify different levels of DM1-J591 or DOTA-J591 conjugates for each identified and measured isoform-of conjugated antibody. In a preferred embodiment this analysis can be performed for samples representing unreacted material, to monitor quality control over time, or resulting from in vitro or in vivo reactions.

In a preferred embodiment direct comparison of unconjugated antibody and conjugated (DOTA, DM1 or DM4) antibody is performed by placing samples in adjacent sample well locations on a MALDI sample plate.

In a preferred embodiment affinity purification using magnetic beads is used to isolate and purify both unconjugated and conjugated antibody from a biological fluid, e.g., plasma or serum, prior to MALDI-TOF MS analysis. In a preferred embodiment molecular weight cutoff spin columns are used to concentrate the affinity purified antibody to allow improved detection in MALDI-TOF MS analysis.

In a preferred embodiment the location of second moiety conjugation sites are identified using affinity tags, e.g., isotope coded affinity tags (ICAT) specific for free sulfhydril (—SH) group, to identify and locate site for loss of second moiety conjugation, e.g., a chelator or toxin, e.g., DM1 or DM4, from antibody molecule. In a preferred embodiment the conjugation sites can be determined for unreacted conjugated sample, to monitor sample stability and identify nature of impurities, and to monitor loss of second moiety conjugation from in vitro or in vivo reactions.

In another aspect, the invention features a method of detecting one or more conjugate-related impurities in a sample. The method includes:

evaluating a sample for the presence or amount of one or more conjugate-related impurities, e.g., a maytansinoid monomer (e.g., a DM1 monomer or a DM4 monomer), a maytansinoid dimer (e.g., a DM1 dimer or DM4 dimer), maytansinoid-TPA adduct (e.g., a DM1-TPA adduct or DM4-TPA adduct), 4-(2-pyridyldithio) pentanoic acid (PPA) and mercaptopyridine, in the sample. The presence of one or more conjugate-related impurities can be indicative of unreacted conjugate and/or linker; breakdown of a conjugated molecule into a first member (e.g., the binding agent, e.g., an antibody or antigen binding fragment thereof) and the conjugate and/or linker for the conjugate; partial breakdown of conjugated molecules resulting, e.g., in changes in conjugation ratios. The evaluation can include comparing the fraction or amount of one or more conjugate-related impurities in the sample with a reference value, e.g., a value from a reference standard, e.g., a value from a standard curve of known concentration of a conjugate-related impurity.

In a preferred embodiment, the evaluation includes separating one or more conjugate-related impurities from the sample. The conjugate-related impurities can be separated by, e.g., chromatography, e.g., high pressure liquid chromatography (HPLC), e.g., reverse phase HPLC. One or more rounds of separation can be performed. A preferred conjugate is a maytansinoid, e.g., DM1, DM4 or a maytansinol. Preferably, the conjugate is DM1 and the conjugate-related impurities are one or more of: DM1 monomer, DM1 dimer, DM1-TPA adduct, 4-(2-pyridyldithio) pentanoic acid (PPA) and mercaptopyridine. Preferably, the conjugate is DM4 and the conjugate-related impurities are analogous to those listed for DM1, e.g., one or more of: DM4 monomer, DM4 dimer, and DM4-TPA adduct.

In a preferred embodiment, the conjugate-related impurities are separated by loading the sample onto a separation matrix, e.g., a column, and using reverse phase HPLC. The separation matrix, provided, e.g., as a column, can include a substrate that retains the conjugate-related impurities for a longer period of time than other sample components, e.g., proteins. For example, the separation matrix can retain smaller molecules (e.g., one or more conjugate-related impurities) while allowing larger molecules (e.g., an antibody and/or antibody conjugate) to pass through the column. In some embodiments, the separation matrix or column allows for differential retention of a conjugate-related impurity from one or more different conjugate-related impurities. For example, in one embodiment, the conjugate is a maytansinoid (e.g., DM1 or DM4) and a maytansinoid monomer is retained on the column for a longer period of time than a maytansinoid dimer. In some embodiments, the conjugate is a maytansinoid (e.g., DM1 or DM4) and a maytansinoid monomer, a maytansinoid dimer, and/or a maytansinoid-linker adduct can be separated from the others. Preferably, the separation matrix is a cross-linked agarose substrate, e.g., a sepharose substrate, e.g., a sepharose substrate. Preferably the matrix is in the form of beads or granules. The separation matrix, particularly if in the form of a bead or other particle, can have a hydrophilic surface and a hydrophobic center. In one embodiment, the hydrophilic surface of the substrate is polyethylene oxide (or a functionally equivalent group, e.g., a group conferring a similar level of hydrophilicity) and/or the hydrophobic center includes hydrophobic phenyl groups (or a functionally equivalent group, e.g., a group conferring a similar level of hydrophobicity). The substrate can have a particle size of about 2.5 μm to about 10 μm, e.g., about 3.0 μm to about 7.5 μm, e.g., about 4.0 μm to about 6 μm, e.g., about 5 μm, and a pore size of about 90 to 150 Å, e.g., about 100 to 140 Å, e.g., about 110 to 130 Å, e.g., about 120 Å. Preferably, the column is equilibrated with a solution prior to loading the sample. The solution can include, e.g., acetonitrile, methanol, isopropanol, tetrahydrofuran, or trifluoroacetic acid (TFA), or mixtures thereof. The pH of the solution can be, e.g., between about 1 and about 7, e.g., between about 1.5 and about 6, e.g., between about 2 and about 5, e.g., between about 2 and 2.5. In some embodiments, the column is a Hisep™ column (Supelco, Bellefonte, Pa.) or a similar separation device.

A Hisep™ column (Supelco, Bellefonte, Pa.) or a similar separation device is suitable for use e.g., when the conjugate-related impurities are separated by reverse phase HPLC, and the solution is selected from acetonitrile, methanol, isopropanol and combinations thereof, preferably the solution is acetonitrile, e.g., 50% acetonitrile/0.01% TFA. In one embodiment, the method can be used to evaluate the presence or amount of a maytansinoid monomer and/or a maytansinoid dimer and the solution has a pH that allows for the distinction between the monomer and the dimer, e.g., the solution brings the pH to about 1 to 4, preferably about 2 to 3. For example, the solution can include acetonitrile (e.g., 50% acetonitrile, e.g., 50% acetonitrile/0.01% TFA).

In a preferred embodiment the sample includes conjugated and unconjugated antibody molecules and the method includes separating substantially all of the conjugated and unconjugated antibody molecules from the sample and detecting conjugate-related impurities remaining in the sample.

The information from the method can be used to determine whether to use (or how to use, e.g., how much to use of) a sample, batch or preparation of conjugated molecule for a preselected purpose, e.g., for formulation, combination with another component, calculation of a dose, measurement or division into doses, e.g., unit doses, packaging, disposal, labeling, administration to a subject, or a decision on whether to execute or perform a second event. Typically, the evaluation can include comparing the fraction or amount of one or more conjugate-related impurity, e.g., a DM1- or a DM4-related impurity, e.g., DM1 monomer, DM4 monomer, DM1 dimer, DM4 dimer, DM1-TPA adduct, DM4-TPA adduct, 4-(2-pyridyldithio) pentanoic acid (PPA) and mercaptopyridine, in the sample with a reference value, e.g., a value from a reference standard, e.g., a value from a standard curve of known concentration of a conjugate-related impurity. If the value for the sample meets a preselected criteria, e.g., it is less than a preselected value, it is used for the preselected purpose.

In a preferred embodiment the method is performed more than once on a sample, and thus allows for the evaluation, e.g., evaluation of the stability of a sample, over time. In such embodiments the method includes:

providing a first portion of the sample at a first point in time, and evaluating it by a method described herein, e.g., separating one or more conjugate-related impurities from the first portion of the sample; and detecting the presence or amount of one or more conjugate-related impurities in the first portion, thereby determining a first level of one or more conjugate-related impurities in the sample; and

providing a second portion of the sample at a second point in time, and evaluating it by a method described herein, e.g., separating one or more conjugate-related impurities from the second portion of the sample; and detecting the presence or amount of one or more conjugate-related impurities in the second portion, thereby determining a second level of one or more conjugate-related impurities in the sample;

thereby evaluating the sample, e.g., evaluating the stability of the conjugated molecules. Evaluating can include comparing one or both the amount detected at the first and second point in time with one or more reference values, e.g., comparing the amount of one or more conjugate-related impurities detected at the first point in time with the amount detected at the second point in time. In a preferred embodiment an increase in the level of one or more conjugate-related impurities between the first portion and the second portion correlates negatively with the stability of the conjugated molecule in the sample. In a preferred embodiment the levels remain the same and the sample is considered stable.

In some embodiments, the first time point occurs at or near a first preselected time or event (e.g., within 1, 5, 10, 24, 48, or 96 hours of the first preselected time or event) and the second time point occurs at or near a second preselected time or event (e.g., within 1, 5, 10, 24, 48, or 96 hours of the second preselected time or event). The first event can be production or purification and the second event can be formulation, combination with another component, calculation of a dose, measurement or division into doses, e.g., unit doses, packaging, disposal, labeling administration to a subject, or a decision on whether to perform or execute a second event. In some embodiments, the first time point is within 1, 5, 10, 24, 48, or 96 hours of purification of the sample, and the second time point is after storage of the sample for 1, 2, 3, 4, 5, 6, 7, 8, 10, or more months, or 1, 2, 3, 4, 5 or more years. In some embodiments, the first time point is after purification of the sample, and the second time point is after formulation of the drug substance.

In some embodiments, the sample includes a biological fluid, e.g., serum, blood, or urine. In other embodiments, the sample is from a batch of conjugated antibody molecules or the sample is a formulated conjugated antibody molecule product. In other embodiments, the sample is a commercially available stock of conjugate.

In some embodiments, the unconjugated antibody molecule is an anti-PSMA antibody molecule, and the conjugated antibody molecule is a conjugated anti-PSMA antibody molecule. In some embodiments, the anti-PSMA antibody molecule is selected from the group consisting of E99, J415, J533, and J591, e.g., deJ591, or antigen binding fragments thereof. In other embodiments, the anti-PSMA antibody is an anti-PSMA antibody described herein.

In some embodiments, the conjugated molecule includes a cytotoxic conjugate, e.g., a compound emitting radiation, molecules of plant, fungal, or bacterial origin, or a biological protein or particle. In particularly preferred embodiments, the cytotoxic conjugate comprises a maytansinoid, e.g., DM1, DM4 or a maytansinol. In some embodiments, the conjugated molecule comprises a detectable conjugate, e.g., biotin or other detectable conjugate described herein.

In some embodiments, separating substantially all of one or more conjugate-related impurities from the sample separates greater than about 90% of the conjugate-related impurity, e.g., greater than about 95%, 97%, 98%, or 99% of the conjugate-related impurity.

In some embodiments, the sample comprises a DM1 or DM4 conjugated J591 antibody molecule, e.g., a DM1 or DM4 conjugated deJ591 antibody molecule, e.g., a formulated DM1 or DM4 conjugated deJ591 antibody molecule also referred to herein as a DS-DM1-deJ591 or a DS-DM4-deJ591 conjugated antibody molecule, respectively.

In some embodiments, the sample comprises less than about 5%, 4%, 3%, 2%, 1%, 0.8%, 0.5% or 0.2% of one or more conjugate-related impurities. In one embodiment, the conjugate is a maytansinoid (e.g., DM1 or DM4) and the sample comprises: less than about 1 μM, 0.5 μM, 0.2 μM or 0.1 μM of maytansinoid monomer; less than about 1.2 μM, 1.0 μM, 0.5 μM or 0.2 μM of maytansinoid dimer; less than about 0.5 μM, 0.2 μM, 0.1 μM or 0.05 μM of maytansinoid-linker adduct (e.g., a maytansinoid-TPA adduct); less than about 1 μM, 0.5 μM, 0.2 μM or 0.1 μM of mercaptopyridine; and/or less than about 1.2 μM, 1.0 μM, 0.5 μM or 0.3 μM of 4-(2-pyridyldithio) pentanoic acid (PPA).

In a preferred embodiment two or more batches or production runs are evaluated, and compared. The method includes providing a first sample from a first batch of conjugated antibody molecules and evaluating it by a method described herein; providing a second sample from a second batch of conjugated antibody molecules and evaluating it by a method described herein; and comparing the results for the two batches.

In another aspect, the invention features, a method of detecting maytansinoid-related impurities in a sample that includes maytansinoid (e.g., maytansinol and more preferably, DM1 or DM4) conjugated anti-PSMA antibody molecules. The method includes evaluating the sample using a method described herein, e.g., separating one or more maytansinoid-related impurities from the sample, e.g., by chromatography; and detecting the presence or amount of one or more conjugate-related impurities in the sample.

In another aspect, the invention features, a method of quantifying maytansinoid-related impurities in a sample that includes maytansinoid (e.g., maytansinol and more preferably, DM1 or DM4) conjugated anti-PSMA antibody molecules. The method includes evaluating the sample using a method described herein, e.g., separating one or more maytansinoid-related impurities from the sample; and detecting the presence or amount of one or more conjugate-related impurities in the sample. In some embodiments, the method can include separating and detecting one or more conjugate-related impurities using chromatography, e.g., HPLC, e.g., reverse phase HPLC.

In another aspect, the invention features a method of evaluating the stability of a sample comprising a maytansinoid conjugated anti-PSMA antibody molecule (e.g., a maytansinol and preferably a DM1 or DM4 conjugated anti-PSMA antibody molecule). The method includes;

providing a first portion of the sample at a first point in time, and evaluating it by a method described herein, e.g., separating one or more maytansinoid-related impurities from the first portion; and detecting the presence or amount of one or more maytansinoid-related impurities in the first portion, thereby determining a first level of one or more maytansinoid-related impurities in the sample; and

providing a second portion of the sample at a second point in time, and evaluating it by a method described herein, e.g., separating one or more maytansinoid-related impurities from the second portion; and detecting the presence or amount of one or more maytansinoid-related impurities in the second portion, thereby determining a second level of one or more maytansinoid-related impurities in the sample;

thereby evaluating the sample, e.g., evaluating the stability of the maytansinoid conjugated anti-PSMA antibody molecule. Evaluating can include comparing one or both the amount detected at the first and second point in time with one or more reference values, e.g., comparing the amount of one or more maytansinoid-related impurities detected at the first point in time with the amount detected at the second point in time. In a preferred embodiment an increase in the level of one or more maytansinoid-related impurities between the first portion and the second portion correlates negatively with the stability of the maytansinoid conjugated anti-PSMA antibody molecule in the sample—a decrease in the amount of maytansinoid conjugated anti-PSMA antibody at the second time points is indicative of a lack of stability. In a preferred embodiment the levels remain the same and the sample is considered stable.

In some embodiments, the first time point occurs at or near a first preselected time or event (e.g., within 1, 5, 10, 24, 48, or 96 hours of the first preselected time or event), and the second time point occurs at or near a second preselected time or event (e.g., within 1, 5, 10, 24, 48, or 96 hours of the second preselected time or event). The first event can be production or purification and the second event can be formulation, combination with another component, calculation of a dose, measurement or division into doses, e.g., unit doses, packaging, disposal, labeling, administration to a subject, or a decision on whether to perform or execute a second event. In some embodiments, the first time point is within 1, 5, 10, 24, 48, or 96 hours of purification of the sample, and the second time point is after storage of the sample for 1, 2, 3, 4, 5, 6, 7, 8, 10, or more months, or 1, 2, 3, 4, 5 or more years. In some embodiments, the first time point is after purification of the sample, and the second time point is after formulation of the drug substance.

In some embodiments, the maytansinoid is DM1.

In some embodiments, the maytansinoid is DM4.

In some embodiments, the method includes separating one or more conjugate-related impurities using chromatography, e.g., high pressure liquid chromatography (HPLC), e.g., reverse phase HPLC.

In some embodiments, the anti-PSMA antibody molecule binds the extracellular domain of PSMA. In some embodiments, the anti-PSMA antibody molecule is E99, J415, J533, or J591, e.g., deJ591, or antigen binding fragments thereof. In some embodiments, the anti-PSMA antibody molecule is an anti-PSMA antibody or antigen binding fragment thereof described herein.

In some embodiments, the method includes comparing the detected conjugate-related impurities to a reference, e.g., a standard curve of known concentrations of conjugate-related impurities.

In another aspect, the invention features a method for evaluating a process for providing a conjugated molecule, e.g., a conjugated antibody, e.g., a process for purifying, conjugating, formulating, or adding a component to, a conjugated molecule. The method includes providing a sample of conjugated molecules made by the process and evaluating it by a method described herein, e.g., separating one or more conjugate-related impurities from the sample, e.g., by chromatography; and detecting the presence or amount of one or more conjugate-related impurities in the sample.

In some embodiments, the method includes comparing the amount of one or more conjugate-related impurities in the sample to a reference value, e.g., the amount of one or more conjugate-related impurities in a sample purified using a different process.

In another aspect, the invention features, a method for evaluating a batch of conjugated antibody molecules. The method includes providing a sample from the batch of conjugated antibody molecules and evaluating it by a method described herein, e.g., separating one or more conjugate-related impurities from the sample, e.g., by chromatography; and detecting the presence or amount of one or more conjugate-related impurities in the sample; and comparing the amount of one or more conjugate-related impurities in the sample to a reference standard, e.g., comparing the amount of one or more conjugate-related impurities in the sample to the amount of one or more conjugate-related impurities in a sample obtained from a different batch, e.g., a reference batch, or to the average amount of one or more conjugate-related impurities in a plurality of samples obtained from more than one batch of the conjugated antibody molecule.

Ranges of conjugate-related impurities described herein can be used, e.g., to evaluate a sample. Thus, the methods described herein can include evaluating whether one or more conjugate-related impurities is present in a range disclosed herein.

A conjugated molecule includes a first member which confers a first property on the conjugated molecule and a second member (referred to sometimes herein as a conjugate) which confers a second property on the conjugated molecule. Typically the conjugated molecule will include a linker moiety which joins the first member to the second member. In a preferred embodiment the first member targets the conjugated molecule or imparts a preselected property with regard to retention time or distribution in a subject.

Exemplary first members include moieties which have a specific affinity for a component found in the subject, e.g., an antibody, or one member of a ligand-receptor pair. Such first members can target the conjugated molecule to a preselected cell type, tissue or organ. The first moiety can possess a property which imparts a preselected property with regard to retention time or distribution in the subject. In preferred embodiments the first member is a ligand, e.g., a naturally occurring ligand, or a soluble form of a naturally occurring ligand, for a receptor found on a target cell. Exemplary receptors include growth factor receptors, hormone receptors, and cytokine, e.g., interleukin, receptors.

In a preferred embodiment the second member provides a therapeutic or diagnostic function. E.g., it can be useful for diagnostics or imaging, e.g., it allows detection of the conjugated molecule, e.g., it emits a signal, or interacts with, e.g., absorbs, light or energy, e.g., X or gamma rays. In other preferred embodiments it is a cell toxin, which can kill or inactivate a cell, e.g., it can be a toxic molecule, e.g., a maytansinoid, e.g., a maytansinol or DM1, or a radionuclide. A preferred conjugated molecule includes as its first member an antibody. The second member, or conjugate, can be, e.g., a toxin, a radioisotope, a chromogen, an enzyme, a fluorochrome, a hapten, or biotin.

As used herein, a “conjugated antibody molecule” includes (i) an antigen-binding polypeptide that is, or is derived from, an antibody or antigen binding fragment thereof, and retains the antigen-binding specificity of the antibody; and (ii) a conjugate, e.g., a toxin, a radioisotope, a chromogen, an enzyme, a fluorochrome, a hapten, or biotin. A number of conjugated antibody molecules are known in the art and include taxane conjugates (see U.S. Pat. No. 6,706,708); anti-VEGF antibody conjugates (see U.S. Pat. No. 6,703,020); anti-CD11a antibodies (see U.S. Pat. No. 6,703,018); gelonin conjugates (see U.S. Pat. No. 6,669,938); and anti-CD33 antibodies (see U.S. Pat. No. 6,599,505). Any conjugate, for which a binding agent is available or can be generated that binds specifically to the conjugate (or a linker used to attach the conjugate to the polypeptide), can be used in the methods described herein.

“Conjugated”, as used herein, refers to an association between a first member and a second member (directly or by way of a linker), e.g., an antibody and a conjugate. Typically it is covalent, but it need not be. Any association, covalent or non-covalent, which provides a conjugated antibody which is suitable for use in treatment is a suitable association. The first member, e.g., an antibody, and conjugate can be linked directly or through a linker. In preferred embodiments the association is not stable in a lysosome.

As used herein, a “binding agent” is an agent that binds with sufficiently high affinity that, in the case of a conjugate-specific binding agent, it is capable of depleting substantially all of the conjugated molecule, e.g., conjugated antibody, in a sample. In the case of a first member specific-binding agent, e.g., an antibody specific-binding agent, it binds with sufficiently high affinity that it allows identification of all or substantially all of the first member, e.g., antibody, in a depleted sample. In a preferred embodiment the affinity of a binding agent has an affinity constant of at least 10⁷ M⁻¹; in some embodiments, the binding agent binds with an affinity constant of 10⁸ M⁻¹ or 10⁹ M⁻¹. In some embodiments, the binding agent is an antibody or antigen binding fragment thereof. In some embodiments, the binding agent is a ligand or other binding partner (e.g., avidin, in the case of biotin). In some embodiments, the binding agent binds directly to the conjugate (e.g., an anti-maytansinoid antibody or antigen-binding fragment thereof).

A “conjugate-specific binding agent”, as used herein, is a binding agent which is sufficiently specific that when contacted with a sample, all or substantially all of it binds only to the conjugate. In preferred embodiments, it binds to the conjugate at least 10², 10³, 10⁴, 10⁵, or 10⁶ fold more than to an antibody in the sample.

A “first member specific binding agent”, e.g., an “antibody-specific binding agent”, as used herein, is a binding agent which is sufficiently specific that when contacted with a sample, all or substantially all of it binds only to the first member, e.g., an antibody. In preferred embodiments, it binds to the antibody at least 10², 10³, 10⁴, 10⁵, or 10⁶ fold more than to a conjugate in the sample.

In some embodiments, the specific binding agent binds to a linker used to attach the conjugate to the first member of the conjugated molecule (e.g., an anti-DOTA antibody or antigen-binding fragment thereof, where the conjugate is coupled to the antibody molecule via a DOTA moiety; or an anti-SPP antibody or antigen binding fragment thereof, where the conjugate is coupled to the antibody molecule via an SPP linker). A conjugate-specific binding agent can bind to any part of a linker as long as that part of the linker remains with the conjugate upon breakdown of the conjugated molecule and release of the conjugate from the first member in the sample. A first member-specific binding agent, e.g., an antibody-specific binding agent, can bind to any part of a linker as long as that part of the linker remains with the first member, e.g., an antibody, upon breakdown of the conjugated molecule and release of the conjugate from the first member in the sample.

As used herein, “antibody molecules” include whole antibodies and antigen binding fragments thereof. Examples of antibody molecules that can be used in the methods described herein include, e.g., monospecific antibody molecules, monoclonal (e.g., human or rodent) antibody molecules, recombinant or in vitro generated antibody molecules, and modified, e.g., chimeric, CDR-grafted, humanized, or deimmunized, antibody molecules.

As used herein, a “formulated product” is a preparation that is in such a form as to permit the active ingredient or ingredients to be therapeutically or prophylactically effective, and that contains no components that are toxic to the subjects to which the formulation is to be administered. Such formulations are known to those skilled in the art.

A “conjugate-related impurity”, as used herein, is a substance derived from a conjugate. A conjugate-related impurity can be, e.g., a process or degradation product of the conjugate, aggregates of the conjugate, e.g., a homodimer or heterodimer, or other chemically derived forms of the conjugate. In the context of a conjugated antibody, a conjugate-related impurity can also include a non-conjugated monomer of the conjugate. For example, a “DM1-related impurity” or “DM4-related impurity”, as used herein, includes DM1 or DM4, e.g., a DM1 monomer or a DM4 monomer; DM1 aggregates or DM4 aggregates, e.g., a DM1 dimer or a DM4 dimer; and process or degradation products of DM1 or DM4, e.g., DM1-TPA adduct, DM4-TPA adduct, 4-(2-pyridyldithio) pentanoic acid (PPA) and mercaptopyridine. In embodiments utilizing a linker described herein, a conjugate-related impurity can also include all, or a portion, of a linker described herein, or a process or degradation product of a linker described herein.

As used herein, the “EC₅₀” is the concentration producing 50% of the response. For this assay the parameter C in the 4 parameter standard curve equation is defined as the EC₅₀.

As used herein, the “lower limit of detection (LLOD)” is the lowest concentration of an analyte for which a response can be reliably distinguished from background.

As used herein, “substantially all” means at least about 90%. In some embodiments, substantially all means that at least about 95%, 97%, 98%, or 99% or more.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic representation of one embodiment of the depletion methods described herein using DS-DM1-deJ591.

FIG. 2 is a line graph showing a standard curve of a depletion using DS-DM1-deJ591.

FIG. 3 is a line graph showing the linearity of DS-DM1-deJ591 standard curve. The binding response and six concentration levels of DS-DM1-deJ591 from 11.25 ng/mL to 0.35 ng/mL were subject to linear regression fit. All parameters pertaining to the linear regression were calculated by using SOFTmax PRO4.0.

FIG. 4A is a line graph showing recovery of deJ591 in DS-DM1-deJ591.

FIG. 4B is a line graph showing recovery of deJ591 in DS-DM1-deJ591 mixed with 1 ng/mL of deJ591 or deJ591 at 1 ng/mL alone after the depletion process.

FIG. 5 is a line graphs showing recovery of deJ591 from DS-DM1-deJ591 at 180, 90 and 45 ng/mL, from 1 ng/mL of deJ591 together with DS-DM1-deJ591 at 180, 90 and 45 ng/mL and from deJ591 at 1 ng/mL alone in 6 experiments performed by two analysts.

FIG. 6 is a line graph showing an overlay comparison of MALDI-TOF mass spectra, displaying doubly-protonated ion mass region, for both unconjugated deJ591 and conjugated DOTA-deJ591. Conjugation levels are labeled from Zero DOTA (peak aligned with centroid of deJ591) to +7 DOTA; mass assignment for Zero DOTA peak is m/z 73,811 (within 5 daltons of measured deJ591, m/z 73,806); mass differences between each DOTA-deJ591 adjacent peak is an average of 518 daltons with a % CV of 3.2%.

FIG. 7 is a line graph showing an overlay comparison of MALDI-TOF mass spectra, displaying doubly-protonated ion mass region, for both unconjugated deJ591 and conjugated DM1-deJ591. Conjugation levels are labeled from Zero DM1 (peak aligned with centroid of deJ591) to +7 DM1; mass assignment for Zero DM1 peak is m/z 73,844 (within 7 daltons of measured deJ591, m/z 73,851); mass differences between each DM1-deJ591 adjacent peak is an average of 826 daltons with a % CV of 4.8%.

FIG. 8 is a line graph showing an overlay comparison of MALDI-TOF mass spectra, displaying doubly-protonated ion mass region, for PK time points 0.25, 1.0, 4.0, 8.0, and 24.0 hrs; changes in average level of DM1:deJ591 conjugation calculated using mass centroid value for each peak is indicated for each time point.

FIG. 9 is a scatter plot showing the Gaussian deconvolution peak-fitting results for mass spectra data shown in FIG. 8; relative abundance for each individual DM1-deJ591 isoform is indicated as present value for each of the PK time points.

FIG. 10 is a panel of the structures of DM1-related impurities.

FIG. 11 is a representative chromatogram of solution containing working concentrations of DM1 monomer, DM1 dimer, DM1-TPA adduct, PPA and mercaptopyridine. Injection volume was 50 μL and peak detection was at 252 nm. Chromatogram is retention time (minutes) versus absorbance (AU).

FIG. 12 is a graph of first-order least squares linear regression analysis of the area response from the DM1 monomer standard solutions. Correlation coefficient (r²), y-intercept, and slope of the regression line are indicated.

FIG. 13 is a graph of first-order least squares linear regression analysis of the area response from the DM1 dimer standard solutions. Correlation coefficient (r²), y-intercept, and slope of the regression line are indicated.

FIG. 14 is a graph of first-order least squares linear regression analysis of the area response from the DM1-TPA adduct standard solutions. Correlation coefficient (r²), y-intercept, and slope of the regression line are indicated.

FIG. 15 is a graph of first-order least squares linear regression analysis of the area response from the PPA standard solutions. Correlation coefficient (r²), y-intercept, and slope of the regression line are indicated.

FIG. 16 is a graph of first-order least squares linear regression analysis of the area response from the mercaptopyridine standard solutions. Correlation coefficient (r²), y-intercept, and slope of the regression line are indicated.

FIG. 17A is a representative chromatogram of DS-DM1-deJ591 at 252 nm.

FIG. 17B is a representative chromatogram of DS-DM1-deJ591 at 280 nm.

FIG. 18 is a graph of first-order least squares linear regression analysis of the area response from the DS-DM1-deJ591 solutions spiked with DM1 monomer. Correlation coefficient (r²), y-intercept, and slope of the regression line are indicated.

FIG. 19 is a graph of first-order least squares linear regression analysis of the area response from the DS-DM1-deJ591 solutions spiked with DM1 dimer. Correlation coefficient (r²), y-intercept, and slope of the regression line are indicated.

FIG. 20 is a graph of first-order least squares linear regression analysis of the area response from the DS-DM1-deJ591 solutions spiked with DM1-TPA adduct. Correlation coefficient (r²), y-intercept, and slope of the regression line are indicated.

FIG. 21 is a graph of first-order least squares linear regression analysis of the area response from the DS-DM1-deJ591 solutions spiked with PPA adduct. Correlation coefficient (r²), y-intercept, and slope of the regression line are indicated.

FIG. 22 is a graph of first-order least squares linear regression analysis of the area response from the DS-DM1-deJ591 solutions spiked with mercaptopyridine. Correlation coefficient (r 2), y-intercept, and slope of the regression line are indicated.

### DETAILED DESCRIPTION

Described herein are methods for quantitatively measuring impurities in a sample that includes antibody molecules, e.g., conjugated antibody molecules. A number of conjugated antibodies are known in the art, as are methods for making such antibodies. For example, conjugated antibody therapeutics include huN901 (BB-10901; ImmunoGen Inc., Cambridge, Mass.) and British Biotech); trastuzumab (Herceptin™; Genentech, Inc, South San Francisco, Calif.); Cetuximab (Erbitux™, ImClone Systems Incorporated, Branchburg, N.J.); Bevacizumab (Avastin™, Genentech, Inc, South San Francisco, Calif.); Edrecolomab (Panorex™, Johnson & Johnson, New Brunswick, N.J.); alemtuzumab (CAMPATH™, Millennium and ILEX Partners, LP, Cambridge, Mass.); gemtuzumab ozogamycin (Mylotarg™, Wyeth-Ayerst Laboratories); ibritumomab tiuxetan (Zevalin™, IDEC Pharmaceuticals Corp, San Diego, Calif.), rituximab (Rituxan™, Genentech, Inc, South San Francisco, Calif.); MDX-210 (Medarex, Princeton, N.J.); G-250 (Wilex AG, Munich, Germany); cantuzumab mertasine (huC242-DM1/SB-408075, ImmunoGen Inc., Cambridge, Mass.); EMD 72 000 (Merck KGaA, Darmstadt, Germany); and ABX-EGF (Abgenix, Fremont, Calif.). Dozens more are in development and testing. Due in part to their biologic nature, strict quality control and testing of the formulated products for each of these therapeutics and potential therapeutics is crucial to the therapeutic effectiveness, dosing and consistency between batches.

In some embodiments, the conjugated antibody is an anti-prostate specific membrane antigen (PSMA) antibody, as described herein. DM1-deJ591 is a conjugated antibody drug product composed of two intermediates: drug maytasinoid-1 (DM1) and an anti-PSMA antibody, deimmunized J591 (also known as deJ591). DM1, an analog of the well characterized cytotoxic drug, maytansine, is conjugated with deJ591 via disulfide formation (using N-succinimidyl 4-(2-pyridyldithio) pentanoate (SPP) as a bifunctional cross-linker). While not wishing to be bound by theory it is believed that the DM1 is released at PSMA sites to obliterate various cancers. Release can occur when a conjugated molecule is taken up by a cell and is disposed in a lysosome. Under lysozomal conditions, the S—S bond is cleaved releasing free DM1. The DM1 is toxic to the cell. The binding distribution of DM1 to deJ591 is important for the determination of biotherapeutic efficacy. As described herein, new analytical methods are provided to characterize DM1-deJ591 complexes during the process of biotherapeutic drug development of DS-DM1-deJ591. The depletion ELISA methods described herein can be used to assess the amount of unconjugated deJ591 present in a DM1-deJ591 drug substance and drug product.

Although the assay methods described herein were developed initially for use with DM1-deJ591, one of skill in the art will appreciate that these methods are equally applicable to other conjugated antibodies.

Depletion Assay

The depletion methods described herein typically include two steps, depletion and detection.

In the depletion step, a formulated drug sample including a conjugated antibody molecule (e.g., DS-DM1-deJ591) is subjected to at least one depletion step, typically two or more depletion steps, e.g., two or more consecutive depletion steps. In some embodiments, three, four, five, six or more depleting steps may be utilized. The depletion steps are typically carried out on a solid surface (e.g., beads, slides, microtiter wells, or other solid matrix, e.g., in a column or batch process) that has been coated with a binding agent specific for the conjugate, e.g., an anti-conjugate antibody molecule (e.g., anti-DM1 antibody molecule), to capture both free conjugate and conjugated antibody species (e.g., DM1 related species and DM1-conjugated deJ591). A number of anti-conjugate antibodies are known in the art and are commercially available. Methods for making such antibodies are also known in the art, and typically include immunizing a suitable animal using the conjugate, or a portion thereof, as an antigen. In some embodiments, the antigen is a linker that connects the conjugate itself to the antibody-derived polypeptide (e.g., as is the case with anti-DOTA antibodies, see Perico et al., J Nucl. Med. 42: 1697-1703 (2001). As described herein, DOTA is a chelator moiety used to link radioactive isotopes to peptides). Thus in some embodiments, the anti-conjugate antibody specifically recognizes a linker used to couple the conjugate to the antibody-derived peptide portion of the conjugated antibody, e.g., an anti-DOTA antibody.

The material remaining after the depletion steps is then used to determine if unconjugated antibody molecules are present in the sample. For example, material remaining after the depletion steps (or a portion thereof) can be transferred into the wells of a plate pre-coated with monoclonal anti-idiotypic antibody molecules (e.g., anti-deJ591 idiotypic antibody molecules). The amount of unconjugated antibody molecules (e.g., deJ591) can then be detected. In some embodiments, the amount of unconjugated antibody molecules is detected using a secondary antibody such as an anti-idiotype antibody molecule or an anti-human IgG antibody molecule, e.g., a biotinylated antibody molecule. The remaining material is contacted with a binding agent specific for the antibody molecule, e.g., a binding agent specific for the antibody molecule bound to a solid support. In some embodiments, to verify that there are no (or few) remaining conjugated antibody molecules present in the material remaining after the depletion steps, the material (or a portion thereof) can be contacted with an antibody molecule that detects the conjugate, e.g., an anti-DM1 or anti-DOTA antibody.

The antibodies used for detection can be labeled directly or indirectly, e.g., with a detectable substance to facilitate detection of the bound or unbound binding agent. Suitable detectable substances include, but are not limited to, various enzymes, prosthetic groups, fluorescent materials, luminescent materials, paramagnetic (e.g., nuclear magnetic resonance active) materials, chromogenic materials, quantum dots, and radioactive materials. A number of such labels, and methods of use thereof, are known in the art.

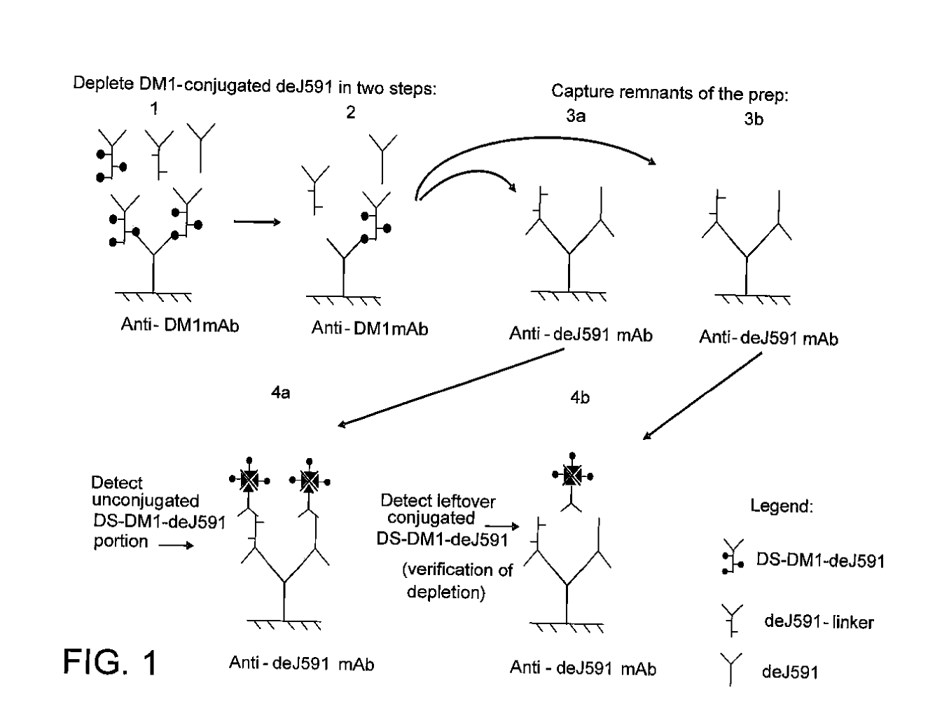

A schematic representation of the depletion methods described herein is shown in FIG. 1 (exemplified using DS-DM1-deJ591). Briefly, DS-DM1-deJ591 is placed into microtiter wells coated with anti-DM1 monoclonal antibody and subjected to two consecutive depletion steps (1, 2). After two depletion steps, the remaining material is transferred to microtiter wells coated with monoclonal anti-deJ591 idiotypic antibody (3a, 3b). The presence of unconjugated deJ591 is detected by using biotinylated donkey anti-human IgG antibody (4a). As a control for satisfactory depletion, biotinylated anti-DM1 antibody is used to detect the presence of DM1-conjugated deJ591, if any (4b). See, e.g., Example 1 herein.

Described herein is a series of experiments to qualify a depletion assay for assessing the percentage of unconjugated antibody (e.g., deJ591) in a conjugated antibody drug substance and drug product (DS-DM1-deJ591, a DM1 conjugated deJ591). The experiments performed to address the depletion aspect of the assay include 1) defining the lower limit of detection using biotinylated murine anti-DM1 antibody and biotinylated donkey anti-human IgG antibody, 2) demonstration of completeness of depletion and 3) demonstration of specificity of depletion and good recovery of spiked deJ591. The experiments conducted for the detection aspect include 1) establishment of standard curve, 2) evaluation of assay accuracy, 3) assessment of intra- and inter-assay precision, 4) determination of the lower limit of quantitation, 5) determination of the lower limit of detection, and 6) determination of linearity. The results together with their respective acceptance criteria are summarized below.

Before evaluating the depletion step, the lower limits of detection of biotinylated anti-DM1 and of donkey anti-human IgG to deJ591 were established. The limits are 0.125 ng/mL as detected by the former and 0.063 ng/mL as detected by the latter.

The depletion process is initiated by placing DS-DM1-deJ591 in microtiter wells coated with anti-DM1 antibody. The absorbance of DM1-deJ591 from 200 ng/mL to 25 ng/mL after the depletion process is less than the mean background (anti-DM1 coated wells containing all reagents except DM1-deJ591)+2 standard deviations, indicating complete removal of DM1 conjugated deJ591. The depletion process is specific for DM1-deJ591 since adding 1 ng/mL of deJ591 into DS-DM1-deJ591 results in a corresponding increase in measured deJ591. In addition, when deJ591 at 1 ng/mL is subjected to the depletion process, the recovery is 0.8 ng/mL measured with coefficients of variation (CVs) ranging from 0% to 6.3%; when deJ591 at 10 ng/mL is subjected to depletion, the recovery is 9.5 ng/mL measured with CVs ranging from 2.7% to 13.5%. Taken together, these results show that the depletion process specifically removes DM1-conjugated deJ591 through a concentration range of 200 to 25 ng/mL without decreasing the level of unconjugated deJ591.

The detection process is initiated by placing assay material into microtiter wells coated with anti-deJ591 monoclonal antibody in the detection plate. deJ591 is used to construct the standard curve used for quantitation because the purpose of this assay is to determine the unconjugated deJ591 in DS-DM1-deJ591 lots. The standard curve is sigmoidal in shape and covers a range from 90 to 0.044 ng/mL. At the linear portion of the curve (11.25 ng/mL to 0.35 ng/mL), the percent CV for each concentration is less than 15%. The mean square of the correlation coefficient (R2) and the mean slope are 0.9999 and 0.994 respectively based on results from 15 experiments. The assay accuracy as measured by evaluating deJ591 at 10 ng/mL (high), 2 ng/mL (medium) and 0.4 ng/mL (low) is well within the acceptance criterion of ±25%. The range of recovery is 80 to 104.5%. The CV of intra-assay precision for 18 determinations has a range of 0.6 to 7.4% which is well below the acceptance criterion of ≦20%. The CV of inter-assay precision for all assays is 3.1%. The lower limit of quantitation is 0.4 ng/mL and the lower limit of detection is 0.063 ng/mL. The amount of unconjugated deJ591 from all experiments (n=12) and all determinations (n=42) has consistently been less than <2% with a range of 0.09 to 1.34%. In addition to meeting the acceptance criteria, these results indicate that the depletion ELISA methods described herein are suitable for determination of the percent unconjugated antibody molecules in drug substance and drug product such as DS-DM1-deJ591.

Anti-PSMA Antibodies

Anti-PSMA antibodies suitable for use in the methods described herein are discussed in this section. In some embodiments, the conjugated antibody molecule includes an antibody that binds the extracellular domain of PSMA as described in U.S. Pat. Nos. 6,107,090, 6,136,311, and 6,649,163; U.S. Patent Application Publication Nos. 2003/0031673, 2003/0161832, 2003/0007974, and 2003/0003101; and copending U.S. patent application Ser. Nos. 10/449,379, 10/379,838, and 10/160,505, all of which are incorporated herein by reference. U.S. patent application Ser. Nos. 10/449,379, 10/379,838, and 10/160,505 describe a deimmunized J591 antibody, referred to herein as deJ591.

Typically, the anti-PSMA antibody interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866).

In some embodiments, the anti-PSMA antibody binds all or part of the epitope of an antibody described in U.S. Pat. Nos. 6,150,508, 6,107,090 and 6,136,311, PCT Publication No. WO 97/35616, PCT Publication No. WO 01/09192, and PCT Publication No. WO 02/098897 (the contents of which are incorporated herein by reference), e.g., one or more of J591, deJ591, E99, J415, J533 or fragments thereof. In other embodiments, the anti-PSMA antibody binds all or part of an epitope recognized by an antibody described in PCT Publication No.: WO 03/064606, U.S. Patent Application Publication No. 2003034903, Schülke et al. (2003) PNAS USA, 100(27):12590-12595; Graver et al. (1998) Cancer Res. 58:4787-4789 (the contents of which are incorporated herein by reference), e.g., one or more of 4A3, 7F12, 8A11, 8C12, 16F9, 026, PSMA 4.40, PSMA 3.7, PSMA 3.8, PSMA 3.9, PSMA 3.11 PSMA 5.4, PSMA 7.3, PSMA 10.3, PSMA 1.8.3, PSMA A3.1.3, PSMA A3. 3.1, Abgenix 4.248.2, Abgenix 4.360.3, Abgenix 4.7.1, Abgenix 4.4.1, Abgenix 4.177.3, Abgenix 4.16.1, Abgenix 30 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153. 1, Abgenix 4.232.3, Abgenix 4.292.3, Abgenix 4.304.1, Abgenix 4.78.1, Abgenix 4.152.1, or fragments thereof.

Conjugates

Conjugates suitable for use in the methods described herein are described in this section. In some embodiments, the conjugate includes a cytotoxic agent or moiety, e.g., a therapeutic drug, a compound emitting radiation, molecules of plant, fungal, or bacterial origin, or a biological protein (e.g., a protein toxin) or particle (e.g., a recombinant viral particle, e.g., via a viral coat protein). For example, the antibody, or antigen-binding fragment thereof, can be coupled to a radioactive isotope such as an α-, β-, or y-emitter, or a β- and γ-emitter. Examples of radioactive isotopes include iodine (¹³¹I or ¹²⁵I), yttrium (⁹⁰Y), lutetium (¹⁷⁷Lu), actinium (²²⁵Ac), praseodymium, or bismuth (²¹²Bi or ²¹³Bi). Radioactive isotopes can be conjugated to the antibody using a linker comprising a chelating agent, e.g., 1,4,7,10-tetraazacyclododecane-N,N′,N″,N′″-tetraacetic acid (DOTA). See, e.g., U.S. patent application Ser. No. 10/449,379. Not all of these species, particularly small radionuclides, will in themselves be antigenic. Thus, they will not be useful with antibody binding agents unless a linker or complexing group can contribute to antigenicity. In such cases the moiety contributing antigenicity would have to remain with the conjugate after breakdown.

The antibody (or antigen-binding fragment thereof) can be coupled to a biological protein, e.g., a molecule of plant or bacterial origin (or derivative thereof), e.g., a maytansinoid (e.g., a maytansinol, DM1 or DM4), as well as a taxane (e.g., taxol or taxotere), or calicheamicin. Other cytotoxic conjugates that can be used include cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof. The conjugate can include, but is not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

The maytansinoid can be, for example, maytansinol or a maytansinol analogue. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. No. 6,333,410, the contents of which is incorporated herein by reference. Maytansinol is describe, e.g., in U.S. Pat. No. 5,208,020 and CC-1065 is described, e.g., in U.S. Pat. Nos. 5,475,092, 5,585,499, 5,846,545.

The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include α₁-BR, α₂-BR, α₃-BR, α₄-BR, β₁-BR, β₂-BR and γ₁-BR. Iodo-complex calicheamicins include α₁-I, α₂-I, α₃-I, β₁-I, β₂-I, δ₁-I and γ₁-BR. Calicheamicin and mutants, analogs and mimics thereof are described, for example, in U.S. Pat. Nos. 4,970,198, issued Nov. 13, 1990, 5,264,586, issued Nov. 23, 1993, 5,550,246, issued Aug. 27, 1996, 5,712,374, issued Jan. 27, 1998, and 5,714,586, issued Feb. 3, 1998, the contents of which are incorporated herein by reference.

Linkers

A linker can be used to couple the conjugate to the antibody molecule. For example, a disulfide linkage can be used, as described in Saito et al., Adv. Drug Delivery Reviews, 55:199-215 (2003); inter alia. Linkers that are sensitive to the lower pH found in endosomes can also be used, including hydrazones, ketals and/or aconitic acids. A hybrid linker can also be used, e.g., a linker with two or more potential cleavage sites, e.g., a disulfide and a hydrazone. Peptidase-sensitive linkers can also be used, e.g., tumor-specific peptidases, for example, linkers sensitive to cleavage by PSA. PEG linkers can also be used (Wüest et al., Oncogene 21:4257-4265 (2002)). Exemplary linkers include hydrazone and disulfide hybrid linkers (Seattle Genetics; see Hamann et al., Bioconjugate Chem. 13:47-58 (2002); Hamann et al., Bioconjug Chem. 13(1):40-6 (2002)); SPP (Immunogen); and a variety of linkers available from Pierce Biotechnology, Inc. In some embodiments, the conjugate, e.g., a maytansinoid, can be coupled to antibodies using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2-iminothiolane, or S-acetylsuccinic anhydride.

Separation Methods

Some of the methods described herein utilize separation of components of a sample described herein, e.g., a biological sample or a stock of a chemical or drug product. Separation techniques that can be used in the methods described herein are known in the art. For example, chromatography achieves the separation of biological or chemical mixtures by the use of a mobile phase and a stationary phase. The mobile phase can be, e.g., a liquid, a gas or a supercritical fluid. The stationary phase can be, e.g., a packed or wall coated standard or capillary column, e.g., a commercially available column such as a Hisep™ column (available from Supelco, Bellefonte, Pa.) or a similar separation device. It is within the skill of those in the art to select particular mobile and stationary phases for a particular application.

The separation matrix is chosen such as to allow the separation of one or more conjugate-related impurity. In this context, separation does not require a high yield separation or purification of the impurity from the sample, but requires such separation or resolution such that the contaminant can be identified and preferably be quantified. In a preferred embodiment the separation matrix can separate one or more and preferably all of a DM1 monomer, a DM4 monomer, DM1 aggregates, e.g., a DM1 dimer, DM4 aggregates, e.g., a DM4 dimer, and process or degradation products of DM1 or DM4, e.g., DM1-TPA adduct, DM4-TPA adduct, 4-(2-pyridyldithio) pentanoic acid (PPA) and mercaptopyridine. Samples such as those described herein can be used to test a separation matrix for suitability.

Preferably, the column is packed with a substrate that allows for small molecules, e.g., molecules of the size of the impurities described herein to be retained on the column while larger molecules, e.g., molecules of about 50,000, 100,00, and more preferably 150,000 daltons or more are not retained. In one embodiment, the substrate is a has a hydrophilic polymer having hydrophobic regions such that smaller molecules can penetrate the hydrophilic network and be retained by the hydrophobic regions while larger molecules do not come into contact with the hydrophobic regions. The size of the particles and the pore size can be determined based upon the size of the antibody and the size of the conjugate-related impurity or impurities. For example, for evaluating maytansinoid-related impurities, the column can have a particle size of about 2.5 μm to about 10 μm, e.g., about 3.0 μm to about 7.5 μm, e.g., about 4.0 μm to about 6 μm, e.g., about 5 μm, and a pore size of about 90 to 150 Å, e.g., about 100 to 140 Å, e.g., about 110 to 130 Å, e.g., about 120 Å. Columns of various lengths and diameters can also be used. In one embodiment, the column is about 5 to 50 cm, e.g., about 10 to 40 cm, e.g., about 20 to 30 cm, e.g., about 25 cm, in length. The diameter of the column can be, e.g., about 1 to 10 cm, e.g., about 2 to 8 cm, e.g., about 3 to 6 cm, e.g., about 4 to 5 cm.

The mixture is injected into the mobile phase and the mobile phase then flows over the stationary phase. The different interactions of the individual components with this combination of phases creates a separation.

Different methods of chromatography are known in the art, e.g., as described in U.S. Pat. No. 5,670,054. For example, separation in a high pressure liquid chromatography (HPLC) column results in an output stream containing a series of regions having an elevated concentration of an individual component of the sample. Each of these regions appear on a chromatogram as a concentration “peak”, and can comprise visible bands within the output stream. There are a number of commercially available detectors that can be used with HPLC, including, e.g., flame ionization detectors; refractive index detectors; fluorescence detectors; UV, visible and IR detectors; and evaporative light scattering detectors (ELSD); all of which can be used in the methods described herein. Quantitation can be performed using known methods in the art, e.g., using commercially-available computer programs. An exemplary system for HPLC consists of a Waters 2695 Separations module with column heater unit, 2996 PDA detector and Millennium Data Chromatography Manager Software, Version 4.0 (available from Meadows Instrumentation, Inc., Zion, Ill.).

### EXAMPLES

The invention is further described in the following examples, which do not limit the scope of the invention described in the claims.

### Example 1

### Evaluation of the Depletion Process

The purpose of the experiments described in this section is to demonstrate the consistency and completeness of removal of DM1 conjugated deJ591 from DS-DM1-deJ591 while providing good recovery of unconjugated deJ591 after the two consecutive depletion steps.

The depletion process is initiated by placing test articles into wells of a microtiter plate that has been coated with anti-DM1 antibody (see FIG. 1). For each assay, the DS-DM1-deJ591 test article is subjected to two consecutive depletion steps. The material remaining after the two depletion steps is transferred to a separate plate coated with monoclonal anti-deJ591 idiotypic antibody. The final material is examined by biotinylated anti-DM1 antibody to ensure the completeness of depletion as well as by biotinylated donkey anti-human IgG antibody to determine the amount of unconjugated deJ591 in the DS-DM1-deJ591 preparation.

Experimentation has established that lots of DS-DM1-deJ591 tested thus far contain <2% unconjugated deJ591. The concentrations of DS-DM1-deJ591 used in the experiments to obtain these results were 225, 75 and 25 ng/mL or 180, 60 and 30 ng/mL. The test method being qualified specifies the latter concentrations.

### 1.1 Lower Limit of Detection (LLOD) for DM1-Conjugated Antibody Detected by Biotinylated Anti-DM1 Antibody and the Lower Limit of Detection for Total Antibody Detected by Biotinylated Donkey Anti-Human IgG Antibody.

After the two depletion steps, biotinylated anti-DM1 antibody is used to detect remaining DM1-conjugated deJ591 whereas biotinylated donkey anti-human IgG antibody is used to detect the presence of deJ591 related species. It is therefore necessary to establish the lower limit of detection (LLOD) when these two antibodies are used as the detecting antibodies.

Experimental design: Low concentrations of DM1-deJ591 at 4, 2, 1, 0.5, 0.25, 0.125, and 0.063 ng/mL were prepared and placed into replicate wells coated with monoclonal anti-deJ591 idiotypic antibody. After incubation and washing steps, the low level of DM1-deJ591 was measured by biotinylated anti-DM1 antibody or by biotinylated donkey anti-human IgG antibody. Three independent experiments were performed.

Acceptance criteria: Report the lowest concentration that consistently produces an optical density (OD) equal to or greater than the mean background+2 standard deviations.

Results and Discussion: The mean background (wells containing no analyte) plus 2 standard deviations from wells detected by biotinylated anti-DM1 antibody is 0.051 OD units. The lowest DM1-deJ591 concentration detected by biotinylated anti-DM1 antibody that consistently produces an OD reading ≧20.051 is 0.125 ng/mL. Thus 0.125 ng/mL is the LLOD for residual DM1-deJ591 after depletion.

For the biotinylated donkey anti-human antibody, the mean background plus 2 standard deviations determined from control wells (wells containing no analyte) is 0.112 OD units. The lowest DM1-deJ591 concentration from all experiments detected by biotinylated donkey anti-human IgG that consistently produces an OD reading ≧0.112 is 0.063 ng/mL. Thus the LLOD of DM1-deJ591 detected by biotinylated donkey anti-human IgG is 0.063 ng/mL.

It is of note that the biotinylated anti-DM1 antibody, a monoclonal antibody, is produced and biotinylated at Millennium Pharmaceuticals, Inc. whereas the biotinylated donkey anti-human IgG, a polyclonal antibody, is purchased commercially. Thus, in addition to the nature of the antibodies, the method of biotinylation, the number of biotin molecules per antibody could very well be different between these two biotinylated antibodies. Furthermore, the respective binding affinity between the two antibodies to the antibody molecule may or may not be the same. These factors certainly could contribute to the overall difference in sensitivity of the two antibodies towards the same analyte.

### 1.2 Completeness of Depletion

Completeness of depletion is defined as total removal of conjugated antibody molecule, e.g., DM1-conjugated deJ591, from a reasonable amount of sample comprising the conjugated antibody molecule after the two depletion steps. The test method being qualified specifies a range of DS-DM1-deJ591 at 180 to 30 ng/mL. To provide for a margin of error, the qualification uses a slightly wider range of 200 to 25 ng/mL.

Experimental design: Two-fold serial dilutions of DS-DM1-deJ591 from 200 to 25 ng/mL were added to replicate wells coated with anti-DM1 antibody. After two cycles of depletion, the remaining material was placed in a new plate coated with monoclonal anti-deJ591 idiotypic antibody to capture remaining unconjugated deJ591 as well as any remaining DM1-conjugated deJ591. The same concentrations of DS-DM1-deJ591 that had not been subjected to the depletion process were placed in the same new plate to serve as positive controls. The presence of DM1-conjugated deJ591, if any, after the depletion process was detected by using biotinylated anti-DM1 antibody. Three independent experiments were performed.

Acceptance criteria: The OD values from all levels of DS-DM1-deJ591 after the depletion process should be <(mean background+2 standard deviation (SD)). OD values from DS-DM1-deJ591 that have not been subjected to the depletion steps are reported. The amount of unconjugated deJ591 from all levels of DS-DM1-deJ591 should not exceed 3%.

Results and Discussion: The OD values from all levels of DS-DM1-deJ591 after the depletion process are shown in Table 1. As shown in Table 1, the OD values from experiments performed by both analysts are all below 0.051 which is mean background+2 SD, indicating complete removal of DM1-conjugated deJ591 by the depletion process. The mean OD after depletion of all experiments at all concentration levels has an OD value of 0.040±0.001. The same concentrations of DS-DM1-deJ591 measured without depletion showed high OD values as expected (Table 2).

After the depletion process, the measured percent unconjugated deJ591 in the DS-DM1-deJ591 reference standard RS-001-01 ranged from 0.99 to 1.34% for the 4 starting concentration levels (Table 3). The average amount of unconjugated deJ591 as detected by two analysts is 1.10% with a SD of 0.0003% (Table 3).

Taken together, the results from these experiments indicate that the depletion process completely removes DM1-conjugated deJ591 without affecting the assessment of unconjugated deJ591. All the results from these experiments meet acceptance criteria.

TABLE 1
Complete removal of DM1-conjugated deJ591 from DS-DM1-deJ591
after the depletion process.

| | Analyst 1 | | | Analyst 2 | | |
|---|---|---|---|---|---|---|
| | Exp't 1 | Exp't 2 | Exp't 3 | Exp't 1 | Exp't 2 | Exp't 3 |
| Concentration | Mean | Mean | Mean | Mean | Mean | Mean |
| (ng/mL) | OD | OD | OD | OD | OD | OD |
| 200 | 0.042 | 0.042 | 0.041 | 0.037 | 0.039 | 0.044 |
| 100 | 0.040 | 0.041 | 0.039 | 0.041 | 0.037 | 0.040 |
| 50 | 0.043 | 0.042 | 0.042 | 0.040 | 0.038 | 0.039 |
| 25 | 0.040 | 0.041 | 0.040 | 0.037 | 0.037 | 0.039 |

| | |
|---|---|
| Background | 0.043 + 2 × 0.004 = 0.051 |
| OD | |
| (0 analyte) ± | |
| 2 SD (n = 6) | |

DS-DM1-deJ591 at 200, 100, 50 and 25 ng/mL was subjected to two cycles of depletion to remove DM1-conjugated deJ591. The remaining material at each concentration level was transferred into wells coated with monoclonal anti-deJ591 idiotypic antibody. Biotinylated anti-DM1 antibody was used to detect the presence of DM1-conjugated deJ591 in the remaining material.

TABLE 2
Without the depletion process, all concentration levels of
DS-DM1-deJ591 exhibit high OD values

| | Analyst 1 | | | Analyst 2 | | |
|---|---|---|---|---|---|---|
| | Exp't 1 | Exp't 2 | Exp't 3 | Exp't 1 | Exp't 2 | Exp't 3 |
| Concentration | Mean | Mean | Mean | Mean | Mean | Mean |
| (ng/mL) | OD | OD | OD | OD | OD | OD |
| 200 | 3.077 | 2.955 | 2.912 | 2.588 | 3.161 | 2.860 |
| 100 | 2.955 | 2.852 | 2.867 | 2.275 | 3.060 | 2.499 |
| 50 | 2.844 | 2.727 | 2.545 | 2.078 | 2.692 | 2.081 |
| 25 | 1.443 | 1.326 | 1.176 | 1.207 | 1.198 | 1.235 |

DS-DM1-deJ591 at 200, 100, 50 and 25 ng/mL was placed in wells coated with anti-deJ591 idiotypic antibody. After incubation and washing steps, the bound DM1-deJ591 was detected with biotinylated anti-DM1 antibody.

TABLE 3
Percent unconjugated deJ591 in DS-DM1-deJ591 after the depletion process

| | % unconjugated deJ591 detected by | | | % unconjugated deJ591 detected by | | |
|---|---|---|---|---|---|---|
| Concentration | analyst 1 | | | analyst 2 | | |
| (ng/mL) | Exp't 1 | Exp't 2 | Exp't 3 | Exp't 1 | Exp't 2 | Exp't 3 |
| 200 | 1.15% | 1.05% | 1.00% | 1.05% | 1.02% | 1.34% |
| 100 | 1.10% | 1.00% | 1.00% | 1.00% | 0.99% | 1.10% |
| 50 | 1.20% | 1.20% | 1.00% | 1.01% | 1.01% | 1.18% |
| 25 | 1.20% | 1.20% | 1.20% | 1.08% | 1.07% | 1.24% |
| Range | 1.10-1.20% | 1.00-1.20% | 1.00-1.20% | 1.00-1.08% | 0.99-1.07% | 1.10-1.34% |
| Overall mean ± | 1.16 ± 0.0005% | 1.11 ± 0.001% | 1.05 ± 0.001% | 1.04 ± 0.0004% | 1.02 ± 0.0003% | 1.22 ± 0.001% |
| SD within | | | | | | |
| day by | | | | | | |
| analyst | | | | | | |

| | | |
|---|---|---|
| Overall mean ± | 1.11 ± 0.06% | 1.09 ± 0.11% |
| SD all exp. | | |
| by analyst | | |
| (n = 3) | | |

| | |
|---|---|
| Overall mean ± | 1.10 ± 0.0003% |
| SD for all | |
| exp. (n = 6) | |

DS-DM1-deJ591 at 200, 100, 50 and 25 ng/mL was subjected to two cycles of depletion to remove DM1-conjugated deJ591. The remaining material at each level was transferred and placed into wells coated with monoclonal anti-deJ591 idiotypic antibody. The percentage of unconjugated deJ591 in the remaining material was detected by biotinylated donkey anti-human IgG antibody and back calculated against the standard curve using SOFTmax PRO 4.0.

### 1.3 Specificity of Depletion and Good Recovery of Spiked DeJ591

Definition: In this ELISA, specificity of depletion implies depletion of antibody that is conjugated, e.g., to DM1 (e.g., DM1-deJ591) while not depleting unconjugated antibody (deJ591). Good recovery of spiked deJ591 is defined by the acceptance criteria.

Experimental design: DS-DM1-deJ591 at 180, 90 and 45 ng/mL, DS-DM1-deJ591 at 180, 90 and 45 ng/mL plus deJ591 at 1 ng/mL at each concentration, and deJ591 at 1 or 10 ng/mL alone were placed into duplicate wells precoated with monoclonal anti-DM1 antibody to deplete DM1-conjugated antibodies. After two cycles of depletion, the material remaining was removed and placed in a new plate coated with monoclonal anti-deJ591 idiotypic antibody. deJ591 from 90 to 0.44 ng/mL was placed in duplicate wells in the same new plate to construct the standard curve. Biotinylated donkey anti-human IgG antibody was used as the detecting antibody. The recovery from all the test articles was back calculated against the standard curve using SOFTmax PRO 4.0.

Acceptance criteria: The amount of unconjugated deJ591 from all concentrations of DS-DM1-deJ591 should not exceed 3% of each level of DS-DM1-deJ591. The % CV for all levels should not exceed 25%. The recovery of deJ591 from DS-DM1-deJ591 spiked with 1 ng/mL of deJ591 should show a proportional increase (total recovery should be within ±50% of expected value). The recovery of the deJ591 at 10 ng/mL should not differ from expected concentration by greater than ±30%.

Results and Discussion: The amount of unconjugated deJ591 recovered from all concentrations of DS-DM1-deJ591 after the depletion process is shown in Table 4. At all concentration levels, the measured amount of recovered unconjugated deJ591 is well below 3%. The range of percent unconjugated deJ591 over the 6 experiments, each containing 3 concentration levels, is 0.9 to 1.1%. The precision for a single measurement ranges from 0.1% to 11.2%. These values are well below the acceptance criterion of ≦75%. There is no significant effect of concentration level on the precision of the measurement within the range studied.

The percent recovery of deJ591 at 10 ng/mL after the depletion process ranges between 86 to 113% (Table 6). The mean recovery is 99.67% for analyst 1 and 90.67% for analyst 2. These recoveries meet the acceptance criterion of ±30% of the expected level (i.e., 70-130%). The precision for the determination has a mean CV of 13.54% for analyst 1 and 2.78% for analyst 2. The overall precision from 6 experiments performed by 2 analysts has a CV of 10.5% with a mean recovery of 95.17% (Table 6).

When 1 ng/mL of deJ591 alone is subjected to the depletion process, in all 6 experiments, the recovery is 0.8 ng/mL (Table 4, last column). When 1 ng/mL of deJ591 is added to 3 concentration levels of DS-DM1-deJ591 before the depletion process, the measured total deJ591 after the depletion process increases as expected (Table 4 and Table 5). The recovery of added deJ591 is calculated by Equation 1.

$$\% recovery = 100 \times ( \frac{( \begin{matrix}measured deJ 591 in spiked sample - \\ measured deJ 591 in unspiked sample\end{matrix} )}{amount of deJ 591 in spike} ) \qquad Equation 1$$

The calculated recovery for the spike ranges from 70 to 100% which is well within ±50% of expected value (Table 5). The precision for recovery of deJ591 at 1 ng/mL has CVs ranging from 0.3 to 3.6% (Table 4). The precision for recovery of total deJ591 in the combination experiments has CVs ranging from 0.0% to 6.3% (Table 4).

The ability to consistently recover the added unconjugated deJ591 indicates that the depletion process is specific. The inability to achieve 100% recovery in most conditions is probably due to reduced retrieval of material after the two depletion steps. In the experiments presented here, the starting volume of analyte used for the depletion process is 100 μL. After two depletion steps the final volume that can be transferred to the detection plate is less than 100 μL. Consequently, the recovery is less than 100%. Based on this finding the test method will be revised to use 110 μL as the starting volume for the depletion process in order to retrieve 100 μL of analyte for analysis in the detection step. This means that the highest DS-DM1-deJ591 concentration used for depletion will be 198 ng. This qualification has demonstrated that complete depletion is obtained with a starting amount of 200 ng [100 μL at 200 ng/mL]. Therefore the change to a starting volume of 110 μL is within the qualified levels for depletion.

TABLE 4
Determination of deJ591 in DS-DM1-deJ591 and recovery of deJ591 after the
depletion process

| | | | | | | |
|---|---|---|---|---|---|---|
| | | | 180 ng/mL | 90 ng/mL | 45 ng/mL | |
| 180 ng/mL | 90 ng/mL | | DS- | DS-DM1- | DS-DM1- | |
| DS- | DS- | 45 ng/mL | deJ591 + | deJ591 + | deJ591 + | 1 ng/mL |
| DM1- | DM1- | DS-DM1- | 1 ng/mL | 1 ng/mL | 1 ng/mL | deJ591 |
| deJ591 | deJ591 | deJ591 | deJ591 | deJ591 | deJ591 | only |

Experiment 1, Analyst 1

| | | | | | | | |
|---|---|---|---|---|---|---|---|
| Amount free | 1.7 | 0.8 | 0.4 | 2.5 | 1.6 | 1.2 | 0.8 |
| deJ591 recovered | | | | | | | |
| (ng/mL) | | | | | | | |
| % of total DS- | 0.9 | 0.9 | 0.9 | N/A | N/A | N/A | N/A |
| DM1-deJ591 | | | | | | | |
| % CV | 0.6 | 2.2 | 0.2 | 1.3 | 0.7 | 0.4 | 1.5 |

Experiment 2, Analyst 1

| | | | | | | | |
|---|---|---|---|---|---|---|---|
| Amount free | 2.0 | 0.9 | 0.5 | 2.7 | 1.7 | 1.3 | 0.8 |
| deJ591 recovered | | | | | | | |
| (ng/mL) | | | | | | | |
| % of total DS- | 1.1 | 1.0 | 1.1 | N/A | N/A | N/A | N/A |
| DM1-deJ591 | | | | | | | |
| % CV | 2.4 | 0.1 | 0.1 | 0.6 | 1.1 | 2.4 | 1.1 |

Experiment 3, Analyst 1

| | | | | | | | |
|---|---|---|---|---|---|---|---|
| Amount free | 2.0 | 0.9 | 0.5 | 2.9 | 1.8 | 1.3 | 0.8 |
| deJ591 recovered | | | | | | | |
| (ng/mL) | | | | | | | |
| % of total DS- | 1.1 | 1.0 | 1.1 | N/A | N/A | N/A | N/A |
| DM1-deJ591 | | | | | | | |
| % CV | 2.4 | 2.6 | 3.7 | 0.8 | 0.0 | 1.4 | 0.3 |

Experiment 1, Analyst 2

| | | | | | | | |
|---|---|---|---|---|---|---|---|
| Amount free | 1.9 | 0.9 | 0.5 | 2.9 | 1.8 | 1.3 | 0.8 |
| deJ591 recovered | | | | | | | |
| (ng/mL) | | | | | | | |
| % of total DS- | 1.1 | 1.0 | 1.1 | N/A | N/A | N/A | N/A |
| DM1-deJ591 | | | | | | | |
| % CV | 1.3 | 0.5 | 1.2 | 0.1 | 0.8 | 0.2 | 1.9 |

Experiment 2, Analyst 2

| | | | | | | | |
|---|---|---|---|---|---|---|---|
| Amount free | 1.8 | 0.9 | 0.5 | 2.8 | 1.7 | 1.3 | 0.8 |
| deJ591 recovered | | | | | | | |
| (ng/mL) | | | | | | | |
| % of total DS- | 1.0 | 1.0 | 1.1 | N/A | N/A | N/A | N/A |
| DM1-deJ591 | | | | | | | |
| % CV | 1.4 | 0.3 | 0.4 | 0.1 | 1.2 | 0.2 | 1.8 |

Experiment 3, Analyst 2

| | | | | | | | |
|---|---|---|---|---|---|---|---|
| Amount free | 1.7 | 0.9 | 0.5 | 2.6 | 1.7 | 1.3 | 0.8 |
| deJ591 recovered | | | | | | | |
| (ng/mL) | | | | | | | |
| % of total DS- | 0.9 | 1.0 | 1.1 | N/A | N/A | N/A | N/A |
| DM1-deJ591 | | | | | | | |
| % CV | 4.5 | 11.2 | 8.2 | 2.9 | 6.1 | 6.3 | 3.6 |

DS-DM1-deJ591 at 180, 90 and 45 ng/mL with or without 1 ng/mL of deJ591 was subjected to two cycles of depletion to remove DM1-conjugated deJ591. The remaining material at each concentration level was removed and placed into wells coated with monoclonal anti-deJ591 idiotypic antibody. The % of unconjugated deJ591 in the remaining material was detected by biotinylated donkey anti-human IgG antibody and back calculated against the standard curve.

TABLE 5
Recovery of 1 ng/mL of deJ591 in three concentration levels of DS-DM1-
deJ591

| | | | | | | |
|---|---|---|---|---|---|---|
| | | | 180 ng/mL | 90 ng/mL | 45 ng/mL | |
| | | | DS-DM1- | DS-DM1- | DS-DM1- | |
| 180 ng/mL | 90 ng/mL | 45 ng/mL | deJ591 + | deJ591 + | deJ591 + | 1 ng/mL |
| DS-DM1- | DS-DM1- | DS-DM1- | 1 ng/mL | 1 ng/mL | 1 ng/mL | deJ591 |
| deJ591 | deJ591 | deJ591 | deJ591 | deJ591 | deJ591 | only |

Experiment 1, Analyst 1

| | | | | | | | |
|---|---|---|---|---|---|---|---|
| deJ591 | 1.7 ng/mL | 0.8 ng/mL | 0.4 ng/mL | 2.5 ng/mL | 1.6 ng/mL | 1.2 ng/mL | 0.8 ng/mL |
| recovered | | | | | | | |
| Without | N/A | N/A | N/A | 0.8 ng/mL | 0.8 ng/mL | 0.8 ng/mL | 0.8 ng/mL |
| deJ591 | | | | | | | |
| from DS- | | | | | | | |
| DM1- | | | | | | | |
| deJ591 | | | | | | | |
| Recovery | N/A | N/A | N/A | 80% | 80% | 80% | 80% |

Experiment 2, Analyst 1

| | | | | | | | |
|---|---|---|---|---|---|---|---|
| deJ591 | 2.0 ng/mL | 0.9 ng/mL | 0.5 ng/mL | 2.7 ng/mL | 1.7 ng/mL | 1.3 ng/mL | 0.8 ng/mL |
| recovered | | | | | | | |
| Without | N/A | N/A | N/A | 0.7 ng/mL | 0.8 ng/mL | 0.8 ng/mL | 0.8 ng/mL |
| deJ591 | | | | | | | |
| from DS- | | | | | | | |
| DM1- | | | | | | | |
| deJ591 | | | | | | | |
| Recovery | N/A | N/A | N/A | 70% | 80% | 80% | 80% |

Experiment 3, Analyst 1

| | | | | | | | |
|---|---|---|---|---|---|---|---|
| deJ591 | 2.0 ng/mL | 0.9 ng/mL | 0.5 ng/mL | 2.9 ng/mL | 1.8 ng/mL | 1.3 ng/mL | 0.8 ng/mL |
| recovered | | | | | | | |
| Without | N/A | N/A | N/A | 0.9 ng/mL | 0.9 ng/mL | 0.8 ng/mL | 0.8 ng/mL |
| deJ591 | | | | | | | |
| from DS- | | | | | | | |
| DM1- | | | | | | | |
| deJ591 | | | | | | | |
| Recovery | N/A | N/A | N/A | 90% | 90% | 80% | 80% |

Experiment 1, Analyst 2

| | | | | | | | |
|---|---|---|---|---|---|---|---|
| deJ591 | 1.9 ng/mL | 0.9 ng/mL | 0.5 ng/mL | 2.9 ng/mL | 1.8 ng/mL | 1.3 ng/mL | 0.8 ng/mL |
| recovered | | | | | | | |
| Without | N/A | N/A | N/A | 1.0 ng/mL | 0.9 ng/mL | 0.8 ng/mL | 0.8 ng/mL |
| deJ591 | | | | | | | |
| from DS- | | | | | | | |
| DM1- | | | | | | | |
| deJ591 | | | | | | | |
| Recovery | N/A | N/A | N/A | 100% | 90% | 80% | 80% |

Experiment 2, Analyst 2

| | | | | | | | |
|---|---|---|---|---|---|---|---|
| deJ591 | 1.8 ng/mL | 0.9 ng/mL | 0.5 ng/mL | 2.8 ng/mL | 1.7 ng/mL | 1.3 ng/mL | 0.8 ng/mL |
| recovered | | | | | | | |
| Without | N/A | N/A | N/A | 1.0 ng/mL | 0.8 ng/mL | 0.8 ng/mL | 0.8 ng/mL |
| deJ591 | | | | | | | |
| from DS- | | | | | | | |
| DM1- | | | | | | | |
| deJ591 | | | | | | | |
| Recovery | N/A | N/A | N/A | 100% | 80% | 80% | 80% |

Experiment 3, Analyst 2

| | | | | | | | |
|---|---|---|---|---|---|---|---|
| deJ591 | 1.7 ng/mL | 0.9 ng/mL | 0.5 ng/mL | 2.6 ng/mL | 1.7 ng/mL | 1.3 ng/mL | 0.8 ng/mL |
| recovered | | | | | | | |
| Without | N/A | N/A | N/A | 0.9 ng/mL | 0.8 ng/mL | 0.8 ng/mL | N/A |
| deJ591 | | | | | | | |
| from DS- | | | | | | | |
| DM1- | | | | | | | |
| deJ591 | | | | | | | |
| Recovery | N/A | N/A | N/A | 90% | 80% | 80% | 80% |

The experiments were performed as described in the legend of Table 4. % Recovery is [(measured deJ591 in spiked sample − measured deJ591 in unspiked sample)/amount of deJ591 in spike] × 100

TABLE 6
Recovery of 10 ng/mL deJ591 after the depletion process

| | Analyst 1 | Analyst 2 |
|---|---|---|
| | % Recovery | % Recovery |
| Experiment 1 | 86 | 93 |
| Experiment 2 | 100 | 91 |
| Experiment 3 | 113 | 88 |
| Mean/analyst (n = 3) | 99.67 | 90.67 |
| RSD/analyst (n = 3) | 13.50 | 2.52 |
| % CV/analyst (n = 3) | 13.54 | 2.78 |
| Overall mean (n = 6) | | 95.17 |
| Overall % CV (n = 6) | | 10.50 |

deJ591 at 10 ng/mL was placed into the depletion plate. After depletion, the remaining material was removed and placed into wells coated with monoclonal anti-deJ591 idiotypic antibody. The amount of deJ591 in the remaining material was detected by biotinylated donkey anti-human IgG antibody and back calculated against the standard curve. % recovery was calculated as (observed value/expected value) × 100.
RSD, relative standard deviation, RSD = SD/mean.

### Example 2

### Evaluation of the Detection Process

The purpose of the detection process in the depletion ELISA is to quantitate the amount of unconjugated antibody, e.g., deJ591, in a conjugated antibody sample, e.g., DS-DM1-deJ591, after removal of the conjugated antibody as well as to ensure the removal process has been completed from the depletion process. This is achieved by placing the remaining material which has been through the depletion process into wells coated with monoclonal anti-deJ591 idiotypic antibody and detecting the material with donkey anti-human IgG antibody. An anti-DM1 antibody may also be used as a control for the depletion process. The optical density readings obtained from the wells detected with donkey anti-human IgG antibody are used to calculate the concentration of deJ591 in DS-DM1-deJ591 based on the deJ591 standard curve which is located in the same plate. The low level of optical density readings (≦background+2 SD) from the wells detected with anti-DM1 antibody is indicative of completeness of removal of DM1-conjugated species. The completeness of depletion and the specificity of depletion have been addressed in Example 1. This example addresses the details of the detection process. All the experiments listed below are performed independently by 2 analysts.

### 2.1 Standard Curve

Definition: The standard curve is the mathematical relationship between the analyte concentration in the standard samples and the binding response. For this ELISA, the standard curve is described by the four-parameter equation:

Y=[(A−D)/(1+(X/C)̂B]+D Equation 2

where Y is the response; X is the concentration; A is the Y-value corresponding to the asymptote at low values of the X-axis; D is the Y-value corresponding to the asymptote at high values of the X-axis; C is the X-value corresponding to the midpoint between A and D; for this assay C is considered to be the EC50. The coefficient B is the slope which reflects how rapidly the curve makes its transition from the asymptotes in the center of the curve.

The standard curve for the depletion ELISA was generated using a specific lot of deJ591 with at least 7 concentration levels to cover a dynamic range of no less than three magnitudes. In addition, concentration levels are placed at the two asymptotic regions of the sigmoidal curve.

Experimental design: Twelve 2-fold serial dilutions of the deJ591 lot starting from 90 ng/mL to 0.044 ng/mL were placed into wells coated with anti-deJ591 idiotypic antibody. After incubation and washing steps, biotinylated donkey anti-human IgG was added to each well. The bound biotinylated antibody was detected by the addition of streptavidin-horseradish peroxidase (HRP) and a chromogenic enzyme substrate solution, TMB (3,3′,5,5′-tetramethylbenzidine). The absorbance was measured at 650 nm. The dose-response relationship is fitted to a 4-parameter equation using SOFTmax PRO4.0. The standard curve in duplicate was run at least three times during three independent days. A 4-parameter logistic model fit to equation 2 above was applied to the concentration-response relationship. The percent CV was determined for each point on each standard curve. The goodness of fit of the data to the calculated curve was represented by the R² for each curve. The mean and the range of the % CVs for each concentration and of the R² for the curves was calculated and reported.

Acceptance criteria: The percent CV of each concentration within the range of antibody concentration within the standard curve should be no more than 15%. The square of the correlation coefficient (R²) for the standard curve should be no less than 0.980, the slope (coefficient B) of the standard curve should be no less than 0.800 and no more than 1.200.

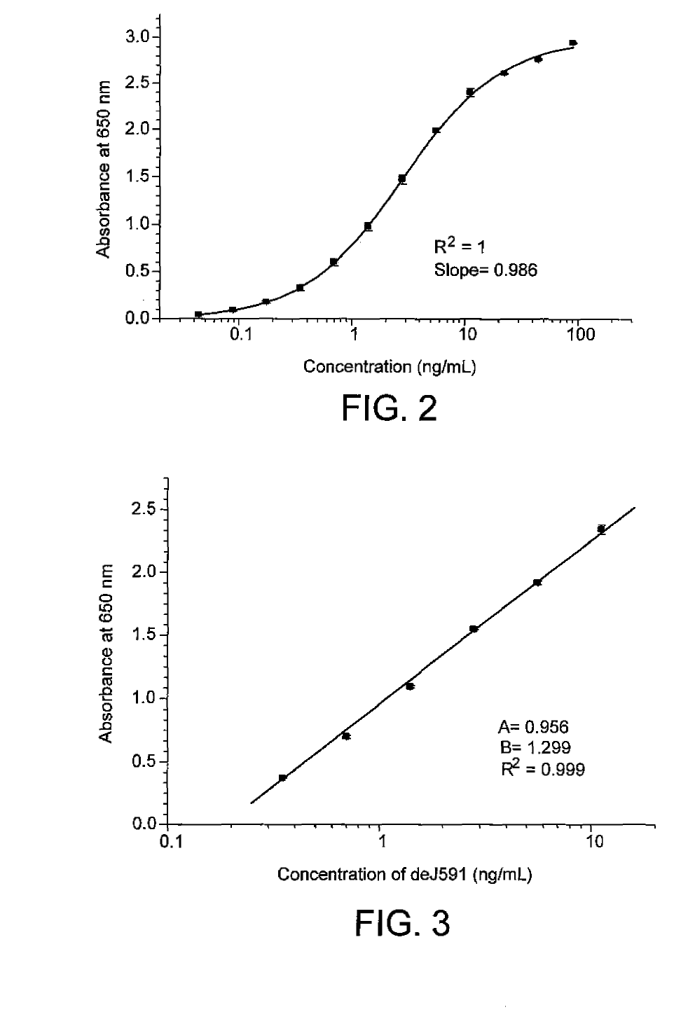

Results and Discussion: The standard curve for this depletion ELISA is constructed with 2-fold serial dilutions of deJ591, starting from 90 ng/mL to 0.044 ng/mL. A representative standard curve which is sigmoidal in shape is shown in FIG. 2. The linear portion of the curve is between 11.25 ng/mL to 0.35 ng/mL (FIG. 3). The % CV of each concentration within the range of quantitation (11.25 to 0.35 ng/mL) is less than 15% with an overall range of 0.3 to 11.4% (Table 8). The % CV of concentration levels located outside the linear range especially levels at the high values on the X-axis are quite high. The overall range of CV from high concentration levels outside of the linear range is 1.0 to 88.1%. The overall range of CV from low concentration levels outside of the linear range is 0 to 16.0% (Table 8). These types of observations are common because the responses outside of the linear portion of the curve are approaching a plateau. The high CVs at the extreme ends are consequences of such responses.

The mean R² and slope (coefficient B) from 15 standard curves are 0.9999 and 0.994 respectively (Table 7). These results meet all acceptance criteria set for the standard curve.

TABLE 7
Slope and R² of deJ591 standard curve

| Experiment No. | Slope | R² |
|---|---|---|
| 1 | 0.924 | 1 |
| 2 | 0.956 | 1 |
| 3 | 0.904 | 1 |
| 4 | 1.016 | 1 |
| 5 | 1.007 | 1 |
| 6 | 0.993 | 1 |
| 7 | 1.007 | 1 |
| 8 | 0.974 | 1 |
| 9 | 1.068 | 0.999 |
| 10 | 1.091 | 1 |
| 11 | 0.964 | 1 |
| 12 | 0.986 | 1 |
| 13 | 0.995 | 1 |
| 14 | 1.051 | 1 |
| 15 | 0.972 | 1 |
| Mean | 0.994 | 0.9999 |
| RSD | 0.05 | 0.0003 |
| % CV | 5 | 0.03 |

The slope and the R² of each standard curve are calculated by SOFTmax PRO 4.0 according to the 4- parameter equation.

TABLE 8
Percent coefficient of variation of 12 concentration levels of standard curve of a
depletion ELISA using DS-DM1-deJ591

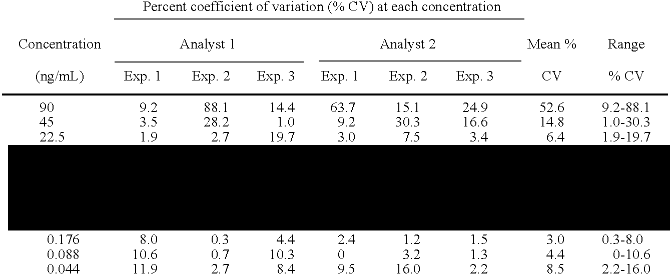

The % CV of each concentration on the standard curve is calculated by using SOFTmax PRO 4.0. The shaded portions denote the concentration levels located on the linear portion of the curve (11.25 to 0.352 ng/mL)

2.1.1 Accuracy for deJ591 Measured without Depletion

Definition: Accuracy reflects the closeness of the observed value to the true value. It is determined by analysis of samples containing known amounts of the analyte. Accuracy is expressed as (observed value/expected value)×100.

Experimental design: Three concentrations, 10 ng/mL, 2 ng/mL, and 0.4 ng/mL of analyte covering the high, medium and low concentrations on the standard curve were prepared in assay diluent and run in triplicate. Three independent assays were performed on 3 separate days.

Acceptance criteria: The mean observed value for each concentration should be within ±25% of the expected value.

Results and Discussion: Table 9 shows the recovery of deJ591 at 10, 2 and 0.4 ng/mL without depletion. The observed value for each concentration level is within 25% (75 to 125%) of the expected value. The lowest recovery among the three concentration levels is from deJ591 at 0.4 ng/mL. This concentration is also the lower limit of quantitation (LLOQ) of deJ591 (section 2.1.4). The mean recovery for all three levels is 87.07% for analyst 1 and 90.21% for analyst 2. The average recovery between the two analysts for all three levels is 88.64%. The overall range of recovery is between 80.0 to 104.5%. When the results are expressed as residuals which are [(observed value−expected value)/expected value]×100, the mean residual from 6 experiments performed by 2 analyst is −11.36% (Table 9). These results meet the acceptance criteria set for accuracy of within ±25% of the expected value.

TABLE 9
Recovery of deJ591 at starting concentrations of 10, 2.0 and 0.4 ng/mL

| | Observed concentration and % recovery by | | | Observed concentration and % recovery by | | |
|---|---|---|---|---|---|---|
| Spiked | Analyst 1 | | | Analyst 2 | | |
| concentration | Experiment 1 | Experiment 2 | Experiment 3 | Experiment 1 | Experiment 2 | Experiment 3 |
| 10 ng/mL | 9.15 (91.5%) | 9.11 (91.1%) | 10.0 (100%) | 10.45 (104.5%) | 9.66 (96.6%) | 9.58 (95.8%) |
| 2.0 ng/mL | 1.66 (83.0%) | 1.62 (81.0%) | 1.84 (92.0% | 1.80 (90.0%) | 1.73 (86.5%) | 1.77 (88.5%) |
| 0.4 ng/mL | 0.34 (85.0%) | 0.32 (80.0%) | 0.32 (80.0%) | 0.33 (82.5%) | 0.33 (82.5% | 0.34 (85.0%) |
| Mean % | 86.50% | 84.03% | 90.67% | 92.33% | 88.53% | 89.77% |
| recovery/exp. | | | | | | |
| Mean % | 87.07% | | | 90.21% | | |
| recovery/ | | | | | | |
| analyst | | | | | | |
| Mean % | 88.64% | | | | | |
| recovery for | | | | | | |
| 2 analysts | | | | | | |
| Mean % | −13.50% | −15.97% | −9.33% | −7.67% | −11.47% | −10.23% |
| residual/exp. | | | | | | |

| | | |
|---|---|---|
| Mean % | −12.93% | −9.79% |
| residual/ | | |
| analyst | | |

| | |
|---|---|
| Mean % | −11.36% |
| residual for 2 | |
| analysts | |

deJ591 at 10, 2 and 0.4 ng/mL were prepared and placed in triplicates into wells coated with anti-deJ591 idiotypic antibody. The recovery from each concentration level is back calculated using SOFTmax PRO 4.0 against the standard curve which was run in the same plate.

2.1.2 Precision for deJ591 Measured without Depletion

Definition: The precision of an analytical method reflects the closeness of individual measures of an analyte when the procedure is applied repeatedly to multiple aliquots of a single homogeneous sample. Precision is usually expressed as % CV. Precision is further divided into intra-assay precision which assesses the variability among replicates on the same plate and inter-assay precision which measures variability among independent experiments.

Experimental design: Three concentrations of analyte at 10 ng/mL, 2 ng/mL, and 0.4 ng/mL were prepared in diluent. Each concentration was run in triplicate for a minimum of 3 times on 3 separate days. The recovery from each concentration level was back calculated against the standard curve which is included in each experiment in the same plate. The % CV from each concentration level was calculated and reported. The % CV for each level from all assays was also calculated and reported.

Acceptance criteria: The intra-assay % CV for all 3 levels should not exceed 20%. As immunoassays are inherently less precise than chromatographic assays, the % CV of inter-assay precision for all concentration levels should not exceed ±30%.

Results and Discussion: The % CV of intra-assay precision for high, medium and low concentration levels are all <20% (Table 10). The range of % CV for intra-assay precision is between 0.5 to 7.4%. The inter-assay precision for the two analysts demonstrates % CVs of 3.7% and 2.7%, respectively (Table 10). The overall % CV for three concentration levels performed six times in triplicate is 3.1%. These results meet all acceptance criteria.

TABLE 10
Precision of deJ591 at 10, 2.0 and 0.4 ng/mL

| Spiked | % CV by Analyst 1 | | | % CV by Analyst 2 | | |
|---|---|---|---|---|---|---|
| concentration | Experiment 1 | Experiment 2 | Experiment 3 | Experiment 1 | Experiment 2 | Experiment 3 |
| 10 ng/ml | 3.3 | 2.2 | 7.4 | 1.0 | 4.5 | 4.8 |
| 2.0 ng/ml | 3.8 | 0.5 | 4.8 | 1.6 | 1.3 | 0.6 |
| 0.4 ng/ml | 4.1 | 1.3 | 5.5 | 4.3 | 3.8 | 2.7 |
| Mean % CV | 3.7 | 1.3 | 5.9 | 2.3 | 3.2 | 2.7 |
| by day | | | | | | |

| | | |
|---|---|---|
| Mean % CV | 3.7 | 2.7 |
| by analyst | | |

| | |
|---|---|
| Mean % CV | 3.1 |
| by two | |
| analyst | |

deJ591 at 10, 2 and 0.4 ng/mL were prepared and placed in triplicates into wells coated with anti-deJ591 idiotypic antibody. The precision from each concentration level was calculated using SOFTmax PRO 4.0 against the standard curve which was run in the same plate.

2.1.3 Lower Limit of Quantitation (LLOQ) of deJ591

Definition: LLOQ is the lowest concentration of the analyte that can be measured with acceptable accuracy and precision.

Experimental design: To determine the LLOQ of deJ591, 5 two-fold serial dilutions of analyte starting from 0.8 ng/mL were prepared. Each concentration was run in triplicate in three separate experiments. After the assays, the accuracy and the precision from each concentration level was calculated.

Acceptance Criteria: The measured value for the lowest concentration should be within 75 to 125% of the expected value. The intra-assay precision at the LLOQ should have a % CV not more than 25%. The inter-assay precision at the LLOQ should have a % CV not more than 30%.

Results and Discussion: The results from 6 experiments performed by two analysts are shown in Table 11. The concentration level that meets the recovery requirement of 75 to 125% of expected value from all experiments is 0.40 ng/mL. For each experiment, the intra-assay % CV of analyst 1 at that concentration level (0.40 ng/mL) is 7.3%, 0.6% or 0.9%. The intra-assay % CV of analyst 2 for the same concentration level from three experiments is 1.3%, 4.4% and 2.2% respectively. The % CV of inter-assay precision for analyst 1 is 2.93% whereas it is 2.70% for analyst 2. Therefore the LLOQ of deJ591 is defined as 0.40 ng/mL.

TABLE 11
Determination of LLOQ of deJ591 standard curve

| | Analyst 1 | | | Analyst 2 | | |
|---|---|---|---|---|---|---|
| Concentration | Obs. Conc. | | | Obs. Conc. | | |
| (ng/mL) | (ng/mL) | % CV | % Recovery | (ng/mL) | % CV | % Recovery |
| | Experiment 1 | | | | | |
| 0.80 | 0.621 | 4.8 | 77.6% | 0.708 | 1.1 | 88.5% |
| 0.40 | 0.308 | 7.3 | 77% | 0.32 | 1.3 | 80.1% |
| 0.20 | 0.149 | 3.6 | 74.5% | 0.152 | 1.9 | 76% |
| 0.10 | 0.075 | 2.5 | 75% | 0.065 | 2.8 | 65% |
| 0.05 | 0.038 | 24.7 | 76% | 0.03 | 6.6 | 60% |
| | Experiment 2 | | | | | |
| 0.80 | 0.655 | 2.8 | 81.9% | 0.612 | 0.9 | 76.5% |
| 0.40 | 0.315 | 0.6 | 78.8% | 0.33 | 4.4 | 82.5% |
| 0.20 | 0.152 | 2.2 | 76.1% | 0.159 | 2.6 | 79.5% |
| 0.10 | 0.073 | 11.5 | 73% | 0.065 | 4.2 | 65% |
| 0.05 | 0.033 | 44.2 | 66% | 0.028 | 5.2 | 46.4% |
| | Experiment 3 | | | | | |
| 0.80 | 0.679 | 0.3 | 84.9% | 0.681 | 2 | 85.1% |
| 0.40 | 0.32 | 0.9 | 80% | 0.316 | 2.2 | 78.9% |
| 0.20 | 0.153 | 3.8 | 76.5% | 0.156 | 2.2 | 78% |
| 0.10 | 0.064 | 7.5 | 64% | 0.084 | 2 | 84% |
| 0.05 | 0.014 | 73.2 | 28% | 0.045 | 4 | 90% |

Microtiter wells were coated with anti-deJ591 antibody. Five 2-fold serial dilutions of deJ591 from 0.80 ng/mL to 0.05 ng/mL were prepared and run in triplicate.

2.1.4 Lower Limit of Detection (LLOD) of deJ591

Definition: LLOD is the lowest concentration of an analyte for which the response can be reliably distinguished from background.

Experimental design: The background OD values from 10 plates were evaluated. The OD from the mean background+2 SD were determined. The value was then interpolated from the standard curve. The average from the 10 interpolated values is the LLOD of the deJ591.

Acceptance criteria: Report value that is near background+2 SD.

Results and Discussion: The mean background OD value from 10 plates plus 2 standard deviations is 0.117. The mean corresponding concentration for the OD value is 0.063 ng/mL

### 2.1.5 Linearity of Standard Curve

Definition: The linearity of an analytical method is its ability to elicit test results that are directly proportional to the concentration of the analyte within a given range.

Experimental design: To establish that there is a linear relationship between the measured concentration and the dilution factor, 3 independent experiments conducted by two analysts were performed in duplicate using serially diluted samples covering at least 5 concentrations.

Acceptance criteria: Report results of R² (square of correlation coefficient), parameter A (y-intercept) and parameter B (slope) of the regression line.

Results and Discussion: As shown in FIG. 3 and in Table 12, deJ591 is linear at concentrations ranging from 11.25 to 0.35 ng/mL. The linear regression line has a mean R² of 0.991±0.006 and a mean slope of 1.32±0.081 (Table 12). The parameter A has a mean of 0.868±0.051. The % CV for all three parameters is <10%. (Table 12).

TABLE 12
Correlation coefficient, y-intercept, and slope of deJ591 from 11.25 to
0.35 ng/mL

| | A (y-intercept | | |
|---|---|---|---|
| Experiment No. | of the line) | B (slope) | R² |
| 1 | 0.899 | 1.420 | 0.996 |
| 2 | 0.956 | 1.299 | 0.999 |
| 3 | 0.843 | 1.402 | 0.987 |
| 4 | 0.819 | 1.198 | 0.977 |
| 5 | 0.854 | 1.307 | 0.992 |
| 6 | 0.836 | 1.295 | 0.997 |
| Mean | 0.868 | 1.320 | 0.991 |
| RSD | 0.051 | 0.081 | 0.008 |
| % CV | 5.88 | 6.14 | 0.81 |

The binding response and six concentration levels of deJ591 from 11.25 ng/mL to 0.0.35 ng/mL were subject to linear regression fit. All parameters pertaining to the linear regression were calculated by using SOFTmax PRO 4.0.

2.2 Accuracy Precision and Linearity of Measuring Unconjugated DeJ591 in DS-DM1-deJ591

The experiments presented in Section 2.1 examined the accuracy, precision and linearity of deJ591 measured in the absence of DS-DM1-deJ591 and without performing the depletion step. Data obtained by measuring the amount of unconjugated deJ591 in the presence of DS-DM1-deJ591 are presented in Tables 4 and 5.

The accuracy of the measurement of unconjugated deJ591 in DM1-deJ591 was further assessed by determining the recovery of added deJ591. One ng/mL deJ591 was added to 180, 90 and 45 ng/mL of DM1-deJ591. Using the value of 1.1% for the mean percent of unconjugated deJ591 in DM1-deJ591 reference standard RS-001-01 (Table 3), the expected amount of deJ591 as calculated by equation 3, is shown in Table 13, row 2.

Expected deJ591=1+(0.011×DS-DM1-deJ591 concentration) Equation 3

Table 14 shows the % recovery that was calculated using equation 4 and the respective expected values of deJ591 from Table 13, row 2 which were calculated using equation 3 above.

$$\% recovery = 100 \times \frac{measured deJ 591}{expected deJ 591} \qquad Equation 4$$

This recovery calculation differs somewhat from that presented in Table 5. The present calculation defines the theoretical value for unconjugated deJ591 in DS-DM1-deJ591 as the mean amount measured in 4 samples each tested in 6 depletion experiments (Table 3). In Table 5, the recovery addresses only the deJ591 added to DS-DM1-deJ591 or in assay diluent only. The recovery in Table 5 was calculated using Equation 1.

$$\% recovery = 100 \times ( \frac{( \begin{matrix}measured deJ 591 in spiked sample - \\ measured deJ 591 in unspiked sample\end{matrix} )}{amount of deJ 591 in spike} ) \qquad Equation 1$$

The recovery of deJ591 after the depletion process in a total of 6 experiments performed by two analysts as calculated by using equation 4 is shown in Table 14. The overall recovery was 90.8%±6.7%. Recoveries ranged from 80.3% to 101.0% for individual assessments. The mean inter-assay precision was 8.5% CV for Analyst 1 and 6.1% CV for Analyst 2. The overall precision for all levels was 7.4% CV with a range of 0.0% to 12.4% CV. There was no significant difference in recovery or precision among the three concentration levels of DS-DM1-deJ591 tested. All results met the acceptance criteria for recovery between 70 and 130% and percent CV of no more than +30% (Table 14).

TABLE 13
Theoretical concentration and measured concentration of deJ591 in DS-DM1-
deJ591 and DS-DM1-deJ591 plus deJ591 or deJ591 alone after the depletion process

| | | | | 180 ng/mL | 90 ng/mL | 45 ng/mL | |
|---|---|---|---|---|---|---|---|
| | 180 ng/mL | 90 ng/mL | 45 ng/mL | DS-DM1- | DS-DM1- | DS-DM1- | 1 ng/mL |
| | DS-DM1- | DS-DM1- | DS-DM1- | deJ591 + 1 ng/mL | deJ591 + 1 ng/mL | deJ591 + 1 ng/mL | deJ591 |
| | deJ591 | deJ591 | deJ591 | deJ591 | deJ591 | deJ591 | only |
| Theoretical | 1.98 | 0.99 | 0.495 | 2.98 | 1.99 | 1.495 | 1 |
| deJ591 | (ng/mL) | (ng/mL) | (ng/mL) | (ng/mL) | (ng/mL) | (ng/mL) | (ng/mL) |
| (ng/mL)* | | | | | | | |

Experiment 1, Analyst 1

| | | | | | | | |
|---|---|---|---|---|---|---|---|
| deJ591 | 1.7 | 0.8 | 0.4 | 2.5 | 1.6 | 1.2 | 0.8 |
| recovered | (ng/mL) | (ng/mL) | (ng/mL) | (ng/mL) | (ng/mL) | (ng/mL) | (ng/mL) |

Experiment 2, Analyst 1

| | | | | | | | |
|---|---|---|---|---|---|---|---|
| deJ591 | 2.0 | 0.9 | 0.5 | 2.7 | 1.7 | 1.3 | 0.8 |
| recovered | (ng/mL) | (ng/mL) | (ng/mL) | (ng/mL) | (ng/mL) | (ng/mL) | (ng/mL) |

Experiment 3, Analyst 1

| | | | | | | | |
|---|---|---|---|---|---|---|---|
| deJ591 | 2.0 | 0.9 | 0.5 | 2.9 | 1.8 | 1.3 | 0.8 |
| recovered | (ng/mL) | (ng/mL) | (ng/mL) | (ng/mL) | (ng/mL) | (ng/mL) | (ng/mL) |
| Mean and | 1.9 ± 0.17 | 0.87 ± 0.06 | 0.47 ± 0.06 | 2.7 ± 0.2 | 1.7 ± 0.1 | 1.3 ± 0.06 | 0.8 ± 0 |
| SD of | (ng/mL) | (ng/mL) | (ng/mL) | (ng/mL) | (ng/mL) | (ng/mL) | (ng/mL) |
| recovered | | | | | | | |
| deJ591 by | | | | | | | |
| analyst 1 | | | | | | | |

Experiment 1, Analyst 2

| | | | | | | | |
|---|---|---|---|---|---|---|---|
| deJ591 | 1.9 | 0.9 | 0.5 | 2.9 | 1.8 | 1.3 | 0.8 |
| recovered | (ng/mL) | (ng/mL) | (ng/mL) | (ng/mL) | (ng/mL) | (ng/mL) | (ng/mL) |

Experiment 2, Analyst 2

| | | | | | | | |
|---|---|---|---|---|---|---|---|
| deJ591 | 1.9 | 0.9 | 0.5 | 2.9 | 1.8 | 1.3 | 0.8 |
| recovered | (ng/mL) | (ng/mL) | (ng/mL) | (ng/mL) | (ng/mL) | (ng/mL) | (ng/mL) |

Experiment 3, Analyst 2

| | | | | | | | |
|---|---|---|---|---|---|---|---|
| deJ591 | 1.7 | 0.9 | 0.5 | 2.6 | 1.7 | 1.3 | 0.8 |
| recovered | (ng/mL) | (ng/mL) | (ng/mL) | (ng/mL) | (ng/mL) | (ng/mL) | (ng/mL) |
| Mean & | 1.8 ± 0.1 | 0.9 ± 0 | 0.5 ± 0 | 2.7 ± 0.15 | 1.7 ± 0.05 | 1.3 ± 0 | 0.8 ± 0 |
| SD of | (ng/mL) | (ng/mL) | (ng/mL) | (ng/mL) | (ng/mL) | (ng/mL) | (ng/mL) |
| recovered | | | | | | | |
| deJ591 | | | | | | | |
| (ng/mL) | | | | | | | |
| by analyst 2 | | | | | | | |
| Mean and | 1.8 ± 0.1 | 0.9 ± 0 | 0.5 ± 0 | 2.8 ± 0.15 | 1.7 ± 0.06 | 1.3 ± 0 | 0.8 ± 0 |
| SD of | (ng/mL) | (ng/mL) | (ng/mL) | (ng/mL) | (ng/mL) | (ng/mL) | (ng/mL) |
| recovered | | | | | | | |
| deJ591 | | | | | | | |
| (ng/mL) | | | | | | | |
| by 2 | | | | | | | |
| analysts | | | | | | | |

Theoretical (or expected) deJ591 is calculated based on the presence of 1.1% of unconjugated deJ591 in DS-DM1-deJ591. When 1 ng/mL of deJ591 was assed to DS-DM1-deJ591, the theoretical deJ591 is calculated by equation 3 where expected deJ591 = 1 + (0.011 × DS-DM1-deJ591 concentration).

TABLE 14
Accuracy and Precision for Measurement of Unconjugated deJ591 in DS-DM1-deJ591 After the Depletion Process

| | | | | | | | | | | | Overall Recovery by | |
|---|---|---|---|---|---|---|---|---|---|---|---|---|
| | Analyst 1 Recovery | | | | | Analyst 2 Recovery | | | | | level (n = 6) | |
| | | | | mean ± | | | | | mean ± | % | mean ± | |
| Test article | Run 1 | Run 2 | Run 3 | SD | % CV | Run 1 | Run 2 | Run 3 | SD | CV | SD | % CV |
| 180 ng/mL DS- | 85.9% | 101.0% | 101.0% | 96.0% ± | 9.1% | 96.0% | 96.0% | 85.9% | 92.6% ± 5.8% | 6.3% | 94.3% ± 6.9% | 7.3% |
| DM1-deJ591 | | | | 8.7% | | | | | | | | |
| 90 ng/mL DS- | 80.8% | 90.9% | 90.9% | 87.5% ± | 6.7% | 90.9% | 90.9% | 90.9% | 90.9% ± 0.0% | 0.0% | 89.2% ± 4.1% | 4.6% |
| DM1-deJ591 | | | | 5.8% | | | | | | | | |
| 45 ng/mL DS- | 80.8% | 101.0% | 101.0% | 94.3% ± | 12.4% | 101.0% | 101.0% | 101.0% | 101.0% ± 0.0% | 0.0% | 97.6% ± 8.2% | 8.4% |
| DM1-deJ591 | | | | 11.7% | | | | | | | | |
| 180 ng/mL DS- | 83.9% | 90.6% | 97.3% | 90.6% ± | 7.4% | 97.3% | 94.0% | 87.2% | 92.8% ± 5.1% | 5.5% | 91.7% ± 5.5% | 6.0% |
| DM1-deJ591 + | | | | 6.7% | | | | | | | | |
| 1 ng/mL deJ591 | | | | | | | | | | | | |
| 90 ng/mL DS- | 80.4% | 85.4% | 90.5% | 85.4% ± | 5.9% | 90.5% | 85.4% | 85.4% | 87.2% ± 2.9% | 3.3% | 86.3% ± 3.8% | 4.4% |
| DM1-deJ591 + | | | | 5.0% | | | | | | | | |
| 1 ng/mL deJ591 | | | | | | | | | | | | |
| 45 ng/mL DS- | 80.3% | 87.0% | 87.0% | 84.7% ± | 4.6% | 87.0% | 87.0% | 87.0% | 87.0% ± 0.0% | 0.0% | 85.4% ± 5.0% | 3.2% |
| DM1-deJ591 + | | | | 3.9% | | | | | | | | |
| 1 ng/mL deJ591 | | | | | | | | | | | | |
| Overall Recovery | | | | 89.8% ± | 8.5% | | | | 91.9% ± 5.6% | 6.1% | | |
| by analyst | | | | 7.6% | | | | | | | | |
| (n = 18) | | | | | | | | | | | | |
| Overall Recovery | | | | 90.8% ± | 7.4% | | | | | | | |
| (n = 36) | | | | 6.7% | | | | | | | | |

Recovery was calculated according to equation 4 where % recovery = 100 × (measured deJ591/expected deJ591)

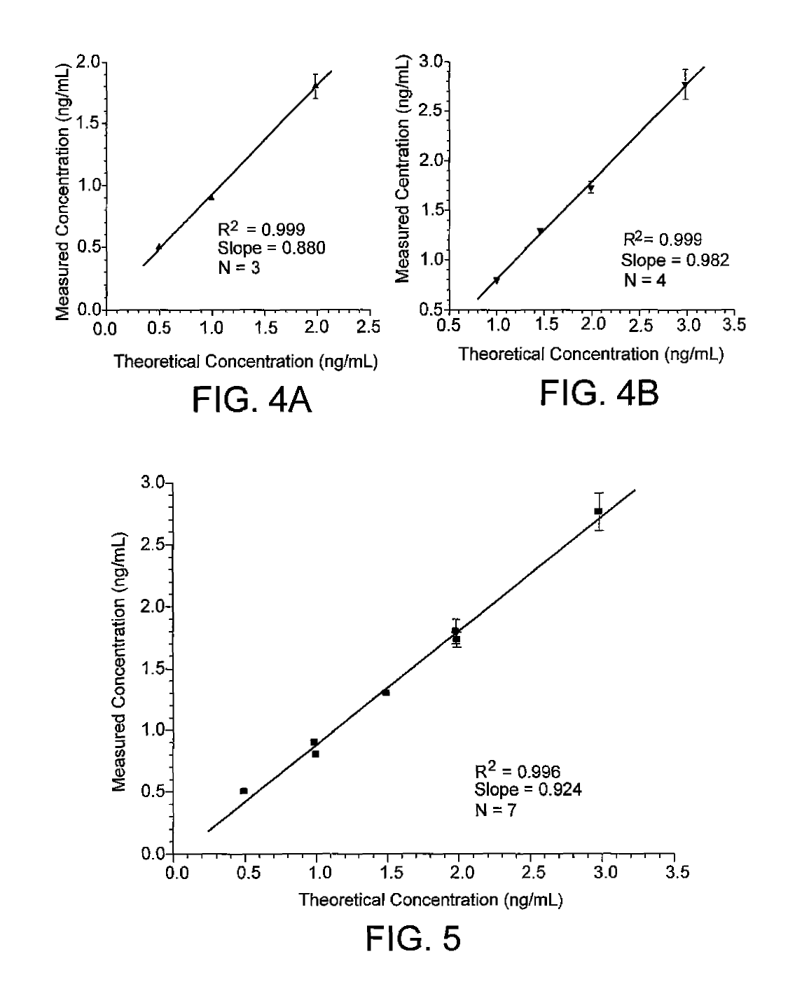

The measured amounts of unconjugated deJ591 in DS-DM1-deJ591 determined in the experiments presented in Table 13 have been used to assess the linearity of the assay by plotting the measured value against the expected value calculated by Equation 3. FIG. 4A plots the data from 6 experiments performed by two analysts. The data clearly fit a linear relationship between measured amount and theoretical amount of unconjugated deJ591 in DS-DM1-deJ591. FIG. 4B shows a linear relationship between measured amount and theoretical amount of free deJ591 in DS-DM1-deJ591 mixed with 1 ng/mL of deJ591 and deJ591 in diluent only from 6 experiments performed by two analysts. FIG. 5 shows that the combined data also fit a linear relationship; the R² for the combined data is 0.996. These results demonstrate that the linearity of the entire assay meets the same criterion as the standard curve. The results of the DS-DM1-deJ591 depletion studies are summarized in Table 15.

TABLE 15
Qualification Summary of DS-DM1-deJ591 Depletion ELISA

| Qualification parameter | Acceptance Criteria | Results |
|---|---|---|
| Lower limit of detection of DS- | Report results | 0.125 ng/mL |
| DM1-deJ591 by anti-DM1- | | |
| biotin | | |
| Lower limit of detection of DS- | Report results | 0.063 ng/mL |
| DM1-deJ591 by anti-human | | |
| IgG-biotin | | |
| Complete removal of DM1- | OD from all concentration levels | All below |
| conjugated deJ591 | after depletion ≦ mean | (mean background + 2 SD) |
| | background + 2SD | |
| Determine the amount of | <3% | All below 3%, with a range of 0.09 |
| unconjugated deJ591 in 200 to | | to 1.34% |
| 25 ng/mL of DS-DM1- | | |
| deJ591after depletion process | | |
| Specificity-recovery of deJ591 | Recovery of 70 to 130% | Recovery of 1 ng/mL deJ591 is |
| at 1 or 10 ng/mL after the | | 80% in all 6 experiments. |
| depletion process | | Recovery of 10 ng/mL deJ591 is |
| | | 90.67% with a range of 86 to |
| | | 113%. |
| Standard curve | CV for each concentration within | CV for concentrations on the |
| | the linear portion should be | linear portion is between 0.3 to |
| | ≦15%, | 11.4%, |
| | R² should be ≧0.980, | mean R² (n = 15) is 0.9999 ± 0.0003, |
| | slope should be ≧0.800 but | mean slope (n = 15) is 0.994 ± 0.05 |
| | ≦1.200 | |
| Assay accuracy at 10, 2 and 0.4 ng/mL | ±25% of expected | All within ±25%; accuracy of −12.04% |
| | | for analyst 1 and of −9.79% |
| | | for analyst 2 with a |
| | | combine range of −20 to 4.5% |
| Assay precision at 10, 2 and 0.4 ng/mL | ≦20% for intra-assay CV, ≦30% | Intra-assay CV is all within 20% |
| | for inter-assay CV | with a range of 0.5 to 7.4%. Inter- |
| | | assay CV is 3.1% |
| Lower limit of quantitation of | Report lowest concentration with | 0.40 ng/mL |
| deJ591 | a recovery of ≧75% and a CV | |
| | of ≦30% | |
| Lower limit of detection of | Report lowest value that is above | 0.063 ng/mL |
| deJ591 | (background + 2 SD) | |
| Assay linearity of the entire | 5 or more concentrations | 7 concentration levels of deJ591 |
| assay | demonstrate a good | demonstrated good linear |
| | concentration-response relation | relationship to expected with an R² |
| | by linear regression fit | of 0996; |
| | CV of each concentration ≦15% | the CV's ranged from 0.0 to |
| | | 11.2% |

### Example 3

### Method for Determine the Percentage of Unconjugated DeJ591 in Drug Substance and Drug Product of DM1-DeJ591 (DS-DM1-DeJ591)

The assay described in this example includes two parts, a depletion part and a detection part. Each part was performed in its respective plate, namely a depletion plate and a detection plate. The assay was performed sequentially starting with the depletion process. After completion of the depletion process, samples to be analyzed were transferred to the detection plate. To allow final transfer of 100 μL of material after being subjected to two consecutive depletion steps, sample solution at 10 μL instead of 100 μL was added into designated wells to initiate the depletion process.

TMB which is stored at 4° C. was brought to room temperate in the dark before use. 11 mL from stock was removed for each microtiter plate and allowed at least 1 hour for warming.

Reagent preparation before assay:

Coating buffer—0.05 M Carbonate-Bicarbonate Buffer in diH2O: One capsule of Carbonate-Bicarbonate Buffer pH 9.6 was dissolved in 100 mL of de-ionized water. The pH was adjusted as necessary. The coating buffer was filtered through a 150 mL 0.22 μm filter system. The coating buffer was stored at room temperature for no more than one month from date of preparation.

Blocking solution/Assay diluent—1% BSA in PBS: 2.5 g of BSA was dissolved in 250 mL PBS and filter through a 250 mL 0.22 μm filter system. The blocking solution/assay diluent was stored at 4° C. for no more than one month from date of preparation.

Wash buffer—0.05% Tween 20 in PBS: 5 mL of Tween 20 was added to 1 L of 10×PBS, and the volume brought up to 10 L with purified de-ionized water collected from Millipore System with resistance of 18 MΩ.

Preparation of Anti-DM1 antibody in coating buffer: This reagent was used as coating reagent for the depletion plate. This procedure was performed two times on the same plate.

Anti-DM1 Antibody in Coating Buffer Preparation:

The volume of anti-DM1 antibody was determined from the stock solution. For this assay, one control and two test articles were run on the same plate. For each depletion step, 16 wells were needed for each sample. Each assay well was coated with 100 μL of anti-DM1 at 10 μg/mL in coating buffer. About 11 mL (11,000 μL) of anti-DM1 in coating buffer was used per plate.

Example:

Starting concentration of anti-DM1 antibody=5.56 mg/mL=5,560 μg/1,000 μL

Required volume of anti-DM1 in coating solution=11,000 μL

Required anti-DM1 concentration=10 μg/mL=10 μg/1,000 μL

X=required volume of anti-DM1 antibody from stock

$$X = \frac{( \begin{matrix}Required volume * \\ Required concentration of anti \text{-} DM 1 antibody\end{matrix} )}{Starting concentration of anti \text{-} DM 1 antibody}$$

$$X = \frac{11 \text{,} 000 μL * 10 μg / 1000 μL}{5560 μg / 1000 μL} = 19.8 μL$$

Round the value to the nearest whole integer Y, Y=20 μL

Calculate required volume of coating buffer Z:

Z=Final volume (11,000 μL)−Rounded volume (Y), Z=11000 μL−20 μL

Z=10,980 μL Coating buffer

Preparation of anti-deJ591 antibody in coating buffer: This reagent was used as coating reagent for the detection plate. The entire plate except wells in column 12 were coated with 100 μL of anti-deJ591 antibody at 10 μg/mL. About 10 mL of anti-deJ591 at 10 μg/mL in coating buffer was used per plate. Anti-deJ591 antibody was diluted from stock solution to 10 μg/mL with coating buffer.

Anti-deJ591 Antibody in Coating Preparation:

Example:

Starting concentration of anti-deJ591 antibody=1.0 mg/mL=1,000 μg/1,000 μL

Required volume of anti-deJ591 in coating buffer=10 mL

=10,000 μL

Required anti-deJ591 concentration=10 μg/1,000 μL

X=required volume of anti-deJ591 from stock

$$X = \frac{( \begin{matrix}Required volume * \\ Required concentration of anti \text{-} deJ 591 antibody\end{matrix} )}{Starting concentration of anti \text{-} deJ 591 antibody}$$

$$X = \frac{10 \text{,} 000 μL * 10 μg / 1000 μL}{1000 μg / 1000 μL} = 100 μL .$$

Calculate required volume of coating buffer Z:

Z=Final volume (10,000 μL)−volume of antibody (100 μL), Z=10,000 μL−100 μL, Z=9,900 μL

A 96 Well Flat Bottom Nunc-Immunoplate MaxiSorb Surface Microplate was coated with 100 μL per well of anti-DM1 antibody at 10 μg/mL in coating buffer and was marked as a depletion plate. A second 96 Well Flat Bottom Nunc-Immunoplate MaxiSorb Surface Microplate was coated with 100 μL per well of anti-deJ591 antibody at 10 μg/mL in coating buffer and marked as a detection plate. Both plates were incubated for one hour in 25°±2° C. incubator.

The plates were then washed once with wash buffer using a microplate washer and the wells blocked with 320 μL of blocking solution per well. The plates were then incubated overnight at 2-8° C.

After overnight blocking, the plates were brought to room temperature (approximately 1 hour or longer).

Preparation of reference samples for depletion: The working concentrations of DS-DM1-deJ591 reference standard and test article(s) were 180, 90 and 45 ng/mL. This was prepared by first performing two serial 1:100 fold dilutions from stock.

Example: Stock concentration of DS-DM1-deJ591-RS-001-01 is 5.2 mg/mL.

Step 1. In an Eppendorf tube, 10 μL of DS-DM1-deJ591 was combined with 990 mL of diluent so that the concentration of the solution was 52 μg/mL=52,000 ng/mL.

Step 2. In an Eppendorf tube, 10 μL of DS-DM1-deJ591 was combined with 990 mL of diluent with 990 mL of diluent. The concentration of the solution was 520 ng/mL=520 ng/1000 μL.

Required starting working DS-DM1-deJ591 concentration was 180 ng/mL. To have enough for serial 2-fold dilutions and for transfer into the wells, 2 mL of 180 ng/mL DS-DM1-deJ591 was prepared.

X=required volume of DS-DM1-deJ591 from Step 2 (520 ng/mL) to prepare 2 mL of DS-DM1-deJ591 at 180 ng/mL

$$X = \frac{( \begin{matrix}Required volume * \\ Required concentration of anti \text{-} DM 1 \text{-} deJ 591\end{matrix} )}{Starting concentration of DS \text{-} deJ 591 \text{-} DM 1}$$

$$X = \frac{2 \text{,} 000 μL * 180 ng \text{/} 1000 μL}{520 ng \text{/} 1000 μL} = 692 μL$$

The required volume of assay diluent Z to bring 2 mL of DS-DM1-deJ591 at 180 ng/mL in assay diluent was calculated:

Z=Final Volume (2,000 μL)−Volume (X)

Z=2,000 μL−692 μL

Z=1,308 μL assay diluent

Two fold serial dilutions of 180 ng/mL of DS-DM1-deJ591 was performed to obtain 90 ng/mL and 45 ng/mL in Eppendorf tubes.

Preparation of test article(s) samples for depletion: The working concentration for the test article(s) were the same as the reference samples at 180 ng/mL, 90 ng/mL and 45 ng/mL These samples were prepared as described above taking into consideration that the stock concentration of each test article may or may not be the same as DS-DM1-deJ591.

Depletion

The blocked depletion plate was washed once with wash buffer using a microplate plate washer and 110 μL of DS-DM1-deJ591 and test article(s) was transferred to the wells.

110 μL of assay diluent was added to some of the wells. The assay plate was covered with plate sealer or plate cover and placed on a plate shaker located in a 25°±2° C. incubator at 500±50 rpm for 1 hour.

The plate was removed from the plate shaker. Multi-channel pipettes were set to 110 μL. Material in column 1 in Table 16 below was transferred to column 7, material from column 2 was transferred to column 8, material from column 3 was transferred to column 9, material from column 4 was transferred to column 10, material from column 5 was transferred to column 11, and material from column 6 was transferred to column 12. The assay plate was covered with plate sealer or plate cover and placed on plate shaker located in a 25°±2° C. incubator.

TABLE 16
Plate design and sample distribution for depletion process

| | | | | | | DS-DM1- | | DS-DM1- | | DS-DM1- | |
|---|---|---|---|---|---|---|---|---|---|---|---|
| DS-DM1- | | DS-DM1- | | DS-DM1- | | deJ591 | | deJ591 | | deJ591 | |
| deJ591 | | deJ591 Test | | Test | | deJ591 | | Test | | Test | |
| Reference | | Article A | | Article B | | Reference | | Article A | | Article B | |
| 1 | 2 | 3 | 4 | 5 | 6 | 7 | 8 | 9 | 10 | 11 | 12 |

| | | | | | | |
|---|---|---|---|---|---|---|
| A | 180 ng/mL | 180 ng/mL | 180 ng/mL | 180 ng/mL | 180 ng/mL | 180 ng/mL |
| B | 90 ng/mL | 90 ng/mL | 90 ng/mL | 90 ng/mL | 90 ng/mL | 90 ng/mL |
| C | 45 ng/mL | 45 ng/mL | 45 ng/mL | 45 ng/mL | 45 ng/mL | 45 ng/mL |
| D | Diluent | Diluent | Diluent | Diluent | Diluent | Diluent |
| E | 180 ng/mL | 180 ng/mL | 180 ng/mL | 180 ng/mL | 180 ng/mL | 180 ng/mL |
| F | 90 ng/mL | 90 ng/mL | 90 ng/mL | 90 ng/mL | 90 ng/mL | 90 ng/mL |
| G | 45 ng/mL | 45 ng/mL | 45 ng/mL | 45 ng/mL | 45 ng/mL | 45 ng/mL |
| H | Diluent | Diluent | Diluent | Diluent | Diluent | Diluent |

| | |
|---|---|
| Depletion step 1 | Depletion step 2 |

During incubation, deJ591 standards were prepared for calibration curve and three levels of deJ591 control samples.

Preparation of calibration curve and control samples: The calibration curve of the detection plate was constructed with twelve 2-fold serially diluted deJ591 starting from 90 ng/mL to 0.044 ng/mL. The control samples for the standard curve are deJ591 at 8, 2 and 0.5 ng/mL.

Preparation of deJ591 Standards:

Example: The stock concentration of deJ591 was 5.2 mg/mL. Due to the high stock concentration, the following steps were performed to prepare a working stock solution of 90 ng/mL.

Step 1. In an Eppendorf tube, 10 μL of deJ591 at 5.2 mg/mL was combined with 990 μL of assay diluent so that the concentration of deJ591 was 52 μg/mL=52,000 ng/mL.

Step 2. In a second Eppendorf tube, 10 μL of deJ591 at 52,000 ng/mL was combined with 990 μL of assay diluent so that the concentration of deJ591 was 520 ng/mL=520 ng/1,000 μL.

Required starting working concentration of deJ591 was 90 ng/mL.

X=Required amount of deJ591 needed from Step 2 to prepare deJ591 at 90 ng/mL

$$X = \frac{( \begin{matrix}Required volume * \\ Required concentration of deJ 591\end{matrix} )}{Starting concentration of deJ 591}$$

$$X = \frac{1 \text{,} 000 μL * 90 ng \text{/} 1000 μL}{520 ng \text{/} 1000 μL} = 173 μL$$

The required volume of assay diluent Z was calculated to bring working stock of deJ591 to 90 ng/mL in assay diluent:

Z=Final Volume (1,000 μL)−Volume (173 μL), Z=1,000 μL−173 μL, Z=827 μL assay diluent

173 μL of deJ591 at 520 ng/mL was combined with 827 μL of assay diluent in an Eppendorf tube. 11 serial 2-fold dilutions were performed to achieve concentrations of 90, 45, 22.5, 11.25, 5.63, 2.81, 1.41, 0.70, 0.35, 0.18, 0.09, and 0.44 ng/mL. The two fold serial dilutions can be carried out either in wells of a U-bottom microtiter plate (110 μL per well to allow transfer of 100 μL per well to an assay plate) or in 11 Eppendorf tubes (500 μL per tube).

Preparation of 3 Levels of deJ591 Control Samples at 8, 2 and 0.5 ng/mL (deJ591 Controls #1, #2 and #3).

The same reagent obtained from Step 2 described above was used.
Example:

The concentration of deJ591 obtained after Step 2 was 520 ng/mL. In an Eppendorf tube, 100 μL of deJ591 at 520 ng/mL was combined with 900 μL of assay diluent so that the concentration of deJ591 was 52 ng/mL=52 ng/1000 μL.

The required starting working concentration of deJ591 for control #1 was 8 ng/mL.

X=Required amount of deJ591 needed from 52 ng/mL to prepare deJ591 at 8 ng/mL

$$X = \frac{( \begin{matrix}Required volume * \\ Required concentration of deJ 591\end{matrix} )}{Starting concentration of deJ 591}$$

$$X = \frac{1 \text{,} 000 μL * 8 ng \text{/} 1000 μL}{52 ng \text{/} 1000 μL} = 153.8 μL$$

X (153.8 μL) was rounded to next integer Y; Y=154 μL

The required volume of assay diluent Z was calculated to bring working stock of deJ591 to 8 ng/mL in assay diluent:

Z=Final Volume (1,000 μL)−Volume (154 μL), Z=1,000 μL−154 μL, Z=846 μL assay diluent

154 μL of deJ591 at 52 ng/mL was combined with 846 μL of assay diluent in an Eppendorf tube.

Two 4-fold serial dilutions were performed to obtain two additional levels of controls, #2 and #3 (2 ng/mL and 0.5 ng/mL respectively). This was performed by transferring 250 μL from concentration above and adding it to a tube containing 750 μL of assay diluent.

The detection plate was washed once with wash buffer using a microplate washer. A multi-channel pipette was set to a volume of 100 μL and was used to transfer from column 7 of the depletion plate to column 1 of the detection plate, column 8 of depletion plate was transferred to wells in column 2 of the detection plate.

TABLE 17
Coating design and sample distribution for detection plate

| | DS-DM1- | | DS-DM1- | | DS-DM1- | |
|---|---|---|---|---|---|---|
| | deJ591 | | deJ591 Test | | deJ591 Test | |
| | Reference | | Article A | | Article B | |
| | 1 | 2 | 3 | 4 | 5 | 6 |
| A | 180 ng/mL | 180 ng/mL | 180 ng/mL | 180 ng/mL | 180 ng/mL | 180 ng/mL |
| B | 90 ng/mL | 90 ng/mL | 90 ng/mL | 90 ng/mL | 90 ng/mL | 90 ng/mL |
| C | 45 ng/mL | 45 ng/mL | 45 ng/mL | 45 ng/mL | 45 ng/mL | 45 ng/mL |
| D | Diluent | Diluent | Diluent | Diluent | Diluent | Diluent |
| E | 180 ng/mL | 180 ng/mL | 180 ng/mL | 180 ng/mL | 180 ng/mL | 180 ng/mL |
| F | 90 ng/mL | 90 ng/mL | 90 ng/mL | 90 ng/mL | 90 ng/mL | 90 ng/mL |
| G | 45 ng/mL | 45 ng/mL | 45 ng/mL | 45 ng/mL | 45 ng/mL | 45 ng/mL |
| H | Diluent | Diluent | Diluent | Diluent | Diluent | Diluent |

Coat wells with anti-deJ591 antibody

| | deJ591 Reference | | | | | |
|---|---|---|---|---|---|---|
| | 7 | 8 | 9 | 10 | 11 | 12 |
| A | 90 ng/mL | 90 ng/mL | 0.35 ng/mL | 0.35 ng/mL | Blank | Empty |
| B | 45 ng/mL | 45 ng/mL | 0.18 ng/mL | 0.18 ng/mL | Blank | Empty |
| C | 22.5 ng/mL | 22.5 ng/mL | 0.09 ng/mL | 0.09 ng/mL | Blank | Empty |
| D | 11.3 ng/mL | 11.3 ng/mL | 0.04 ng/mL | 0.04 ng/mL | Blank | Empty |

| | | | | | |
|---|---|---|---|---|---|
| E | 5.6 ng/mL | 5.6 ng/mL | Control #1 | Blank | Empty |
| | | | 8 ng/mL | | |
| F | 2.8 ng/mL | 2.8 ng/mL | Control #2 | Blank | Empty |
| | | | 2 ng/mL | | |
| G | 1.4 ng/mL | 1.4 ng/mL | Control #3 | Blank | Empty |
| | | | 0.5 ng/mL | | |
| H | 0.7 ng/mL | 0.7 ng/mL | Blank | Blank | Empty |

Coat wells with anti-deJ591 antibody

Briefly, wells, except wells in column 12, were coated with 100 μL of anti-deJ591 antibody at 10 μg/mL. Samples in wells located in rows A to H, columns 1 to 6 were transferred from the depletion plate. Wells located in columns 7 and 8, 9A to 9D, 10A to 10D were used for the standard curve, deJ591 from 90 ng/mL to 0.04 ng/mL. Controls #1, #2, and #3 denote three levels of concentrations of deJ591 at 8, 2 and 0.5 ng/mL respectively. Wells located in H9, H10 and column 11 are designated as blank in the SOFTmax PRO.

The detection plate was placed on a plate shaker located in a 25°±2° C. incubator set at 500±50 rpm for 1 hour.

The following reagents were prepared:

Biotinylated mouse anti-DM1 antibody. Biotinylated anti-DM1 antibody was used at 1:20,000 dilution (1:20 K dilution). The dilution was made by first combining 5 μL of stock solution with 4995 μL of assay diluent in a 15 mL conical tube to obtain a 1:1,000 dilution. After thorough mixing, 0.5 mL of diluted anti-DM1 biotin was removed and mixed with 9.5 mL of diluent. The final dilution was 20,000 fold (1:20 K).

Biotinylated donkey anti-human IgG antibody. This antibody was used at 1:50,000 dilution (1:50 K). The dilution was made by first combining 5 μL of stock solution with 4,995 μL of assay diluent in a 15 mL conical tube to obtain a 1:1,000 dilution. After thorough mixing, 0.5 mL of diluted anti-human IgG biotin was removed and mixed with 24.5 mL of diluent. The final dilution was now 50,000 fold (1:50 K).

The plate was removed from the incubator and washed three times with wash buffer using a microplate washer. 100 μL of biotinylated mouse anti-DM1 antibody was pipetted into wells located from rows A to D and columns from 1 to 6. 100 μL of biotinylated donkey anti-human IgG was pipetted into all the remaining wells, except wells located in column 12. The plate was placed on a plate shaker located in a 25°±2° C. incubator and set at 500±50 rpm for 1 hour. The plate was washed three times with wash buffer using a microplate washer. A 1:50,000 dilution of streptavidin-HRP solution was prepared by combing 5 μL of streptavidin with 4,995 μL of assay diluent. After thorough mixing, 0.5 mL was removed and added to 24.5 mL of assay diluent. The diluted streptavidin-HRP was placed in a reagent reservoir and then delivered at 100 μL/well of streptavidin to all wells from row to row except wells located in column 12. The plate was covered and incubated at 25° C. with shaking for 30 to 35 minutes. The plate was then washed three times with wash buffer using a microplate plate washer and room temperature TMB was poured into a reagent reservoir. 100 μL/well of TMB was dispensed from row to row for all the desired wells and the plate incubated at room temperature or at 25°±2° C. in the dark for 15±2 minutes. SOFTmax PRO4.0 was then set at 650 nm and automix for 5 seconds and the absorbance read at 650 nm using a microplate spectrophotometer. To determine the % unconjugated deJ591 in samples of DS-DM1-deJ591, a column was added in the display by creating % deJ591 with the formula “(Mean Result/Concentration)×100” and the % unconjugated deJ591 from three concentration levels of DS-DM1-deJ591 (180, 90 and 45 ng/mL) was averaged. For the three levels of deJ591 controls, a column was created to include accuracy with the formula “(Mean Result/Concentration)×100”.

Assay Acceptance Criteria: 1) % CV of each concentration on the linear portion of the standard curve (11.25 to 0.35 ng/mL) should be ≦15%; 2) R² value for the entire curve should be no less than 0.980; 3) the accuracy of each control level should be within ±25% of the expected value; 4) the CV of each control level should be ≦20%. The OD values from all levels of DS-DM1-deJ591 after the depletion process should be ≦(mean back ground+3 standard deviations). In this context, background consists of wells coated with anti-DM1 antibody, containing blocking solution, biotinylated anti-DM1 antibody, streptavidin-HRP and TMB but no DS-DM1-deJ591 test article. The percentage of unconjugated deJ591 in DS-DM1-deJ591 reference standard should be ≧0.5% but ≦4%.

### Example 4

### Method for Measuring Free deJ591 in Human Serum

The methods described in this example were developed to measure free deJ591 antibody (also known as naked deJ591 or unconjugated deJ591) in human serum after a patient has been administered DS-DM1-deJ591. The methods include a sample pre-treatment step in which deJ591-DM1 is separated from free deJ591 by incubating the sample in anti-DM1 coated microtiter plate wells. The supernatant containing free deJ591 is then assayed using the assay for total deJ591. Since the anti-DM1 coated wells have a limited binding capacity, the separation step is performed twice to ensure complete removal of deJ591-DM1. The method described below is for one sample dilution, but can be adapted for use with two or more dilutions.

For each sample to be assayed, 4 microtiter plate wells were coated with 100 μL anti-DM1 antibody at a concentration of 10 μg/ml in carbonate buffer.

The plate was sealed and incubated at room temperature for 60±10 minutes.

While the plate was coating, each serum sample was diluted to a concentration of approximately 20 ng/ml in a volume of 0.5 ml. The anti-DM1 coated plate was washed and blocked with 150 μl of 5% nonfat dry milk in PBS (blocking buffer). The plate was sealed and incubated at room temperature for 60±10 minutes.

While the plate was blocking, plates, standards and controls for the detecting total deJ591 were prepared using anti-deJ591 (anti-idiotype) coated plates. The anti-DM1 coated plate was washed and 100 μl of each diluted sample was added to two of the four anti-DM1 coated wells. The other two wells were reblocked with 150 μl blocking buffer; and used for the second separation. The plate was sealed and incubated at room temperature for 60±10 minutes on a plate shaker.

Using an 8-position manifold and vacuum aspirator, the blocking buffer was aspirated from each well, then each set of 2 samples from the first set of anti-DM1 wells was transferred into those wells, using an 8-channel multi-pipette. The plate was sealed and incubated at room temperature for 60±10 minutes on a plate shaker.

The treated samples, plus standards and controls, were transferred into the plate prepared for the detection of total deJ591. The detection of total deJ591 was performed and the concentration of free deJ591 was calculated.

### Example 5

### The Use of Pyridyl Disulfide for Rapid Capture and Sensitive Quantification of Free DM1 in Biological Matrices

The methods described in this example are useful for quantifying DM1 or any molecule which possesses a free thiol group in a biological fluid, including animal and human serum/plasma and urine.

Free DM1 is a highly potent cytotoxin which is nondiscriminatory against both malignant and healthy cells. In order to improve the specificity of DM1 and reduce its potential toxicity, a conjugate (e.g., DM1-deJ591) has been recently developed.

The DM1-deJ591 linkage is a disulfide bond which is expected to be stable in circulation and to dissociate within targeted cancer cells to release free DM1 within the cell in order to kill the targeted cancer cell. Monitoring the extracellular free DM1 concentration is important in order to assess the potential efficacy and toxicity of a DM1 conjugated antibody molecule which might unexpectedly release free DM1 in circulation.

However, direct detection and quantification of DM1 in serum/plasma are difficult due to the reactivity of free DM1 with biological matrices, leading to its rapid loss after sample collection. In addition, the lack of a charge center in the DM1 molecule leads to low sensitivity in DM1 quantification even when using highly sensitive LC/MS/MS based techniques.

In order to solve the problem, a methodology has been developed that uses pyridyl disulfide for rapidly capturing free DM1 from biological samples such as serum, plasma or urine to form a more stable derivatization product (DM1-PDS). A polar pyridine group is incorporated into the derivative which significantly improved the sensitivity for quantification by at least 30 fold. The sensitivity improvement makes it possible to detect free DM1 at a concentration lower than its IC₅₀ towards cells.

### Example 6

### MALDI-TOF Mass Spectroscopy Methods for Characterizing Conjugated Antibodies Such as DOTA-deJ591 and DM1-deJ591

Since the conjugation reaction between either DM1 and deJ591 or DOTA and deJ591 results in a heterogeneous mixture, representing various levels of conjugation, the number of DM1 molecules conjugated to deJ591 or the number of DOTA molecules conjugated to deJ591 needs to be accurately determined. Recent advances in MALDI-TOF MS have provided a novel analytical technique capable of detecting and characterizing macromolecules (Siegel et al., Calicheamicin Derivatives Conjugated to Monoclonal Antibodies: Determination of Loading Values and Distributions by Infrared and UV Matrix-Assisted Laser Desorption/Ionization Mass Spectrometry and Electrospray Ionization Mass Spectrometry Anal. Chem. 1997, 69, 2716-2726; Guidance for Industry, Bioanalytical Method Validation by FDA; available on the world wide web at fda.gov/cder/guidance/index.htm). The MALDI-TOF MS analytical strategy described herein focuses on resolving and identifying the different masses of deJ591 representing various levels of DM1 or DOTA conjugation. The use of MALDI-TOF MS provides the advantage of using an uncomplicated and rapid bimolecular measurement, as opposed to LC/ESI-MS, to characterize both DOTA and DM1 distribution ratios to deJ591, especially when compared with other analytical methods requiring separation coupled with quantitation for each individual level of conjugation.

A MALDI-TOF mass spectrometer (Voyager Elite, Applied Biosystem, Framingham, Mass.) was used to determine the level of DOTA conjugation to deJ591 before and after quality control of DOTA-NHS starting material. The unconjugated deJ591 and deJ591-DOTA complex were provided in a solution of 0.3M ammonium acetate buffer, pH 6.8, at an antibody concentration of approximately 5 to 10 mg/mL. The deJ591-DOTA was desalted using G25 UltraMicroSpin column (Nest Group, Inc.) by first adding 200 uL of milliQ water to the column and spinning at 5000 rpm for 3 min, transferring the column to a new collection tube, loading 25 uL of the deJ591-DOTA containing solution to bed of column, spinning at 5000 rpm for 3 min. The resulting purified sample was eluted into a collection tube and this sample was then concentrated to approximately 0.5 to 1.0 mg/mL of antibody by addition of milliQ water with 0.1% trifluoroacetic acid. Sinapinic acid at 10 mg/mL in 50/50 ACN/H2O with 0.1% TFA was used as MALDI matrix. A volume ratio of 1:1 for sample to matrix was used and 1 μL of this mixture was deposited to the MALDI sample stage. The MALDI-TOF mass spectrometer was operated in positive ion, delayed extraction, mode using a nitrogen laser (337 nm; 100-μm spot size) pulsed at 4 Hz; each mass spectra is the result of 200 laser pulses. External calibration was performed using single and double charge state peaks from a Bovine Serum Albumin standard, resulting in an average of +/−0.1% mass assignment across sample plate. Direct comparison of unconjugated deJ591 and conjugated (DOTA or DM1) deJ591 was performed by placing samples in adjacent sample well locations on a MALDI sample plate, providing optimal reproducibility of mass measurements of less than +/−0.05%. Distribution ratios for conjugation of either DOTA or DM1 to deJ591 were determined using the doubly-protonated (2+ charge state) mass spectral peak for the conjugated antibody, followed by data processing using Gaussian deconvolution and peak fitting software (PeakFit, Systat, Inc., Richmond, Calif.).

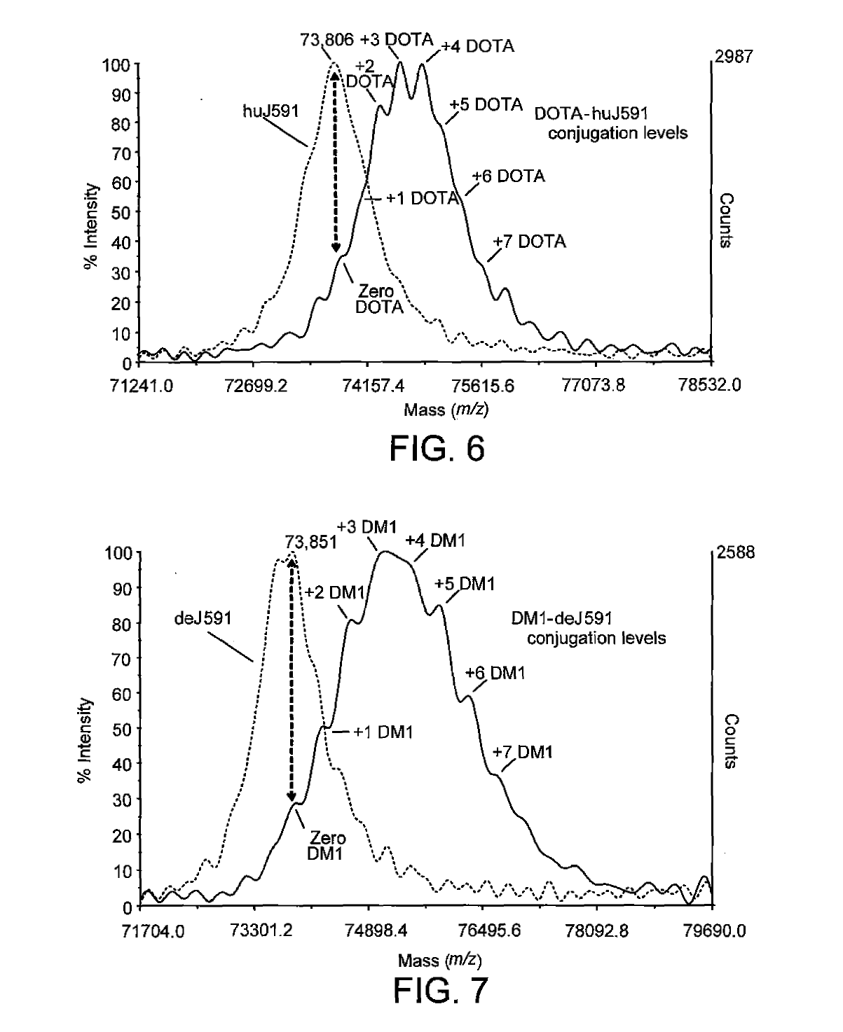

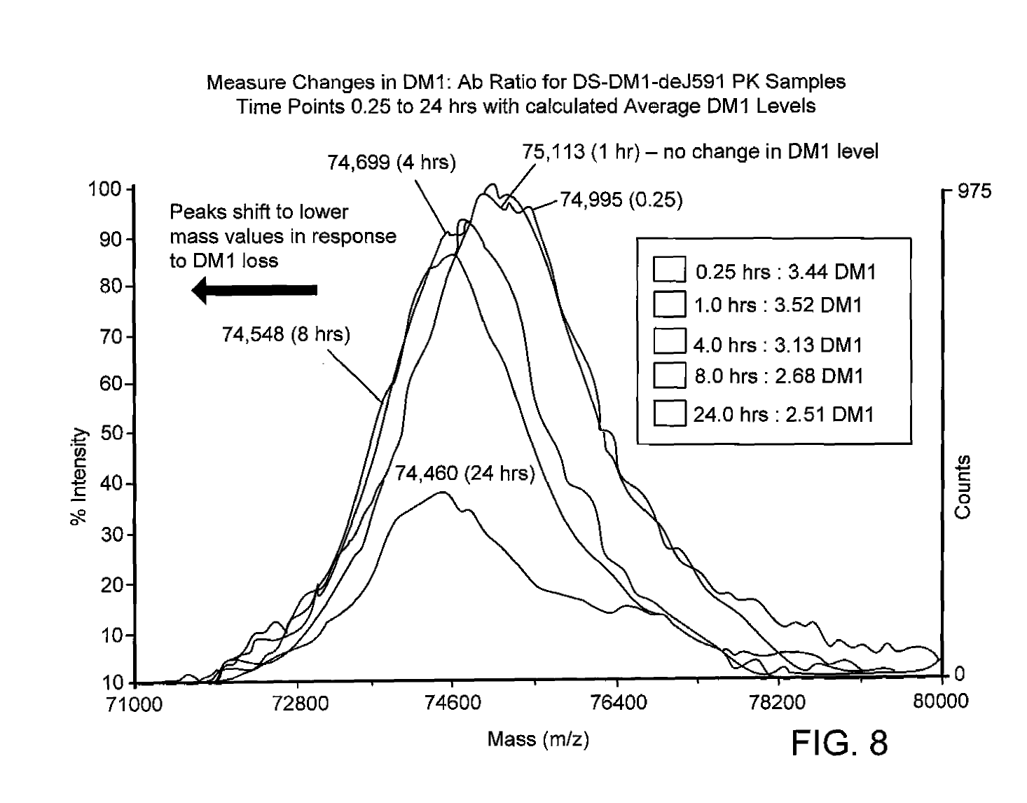

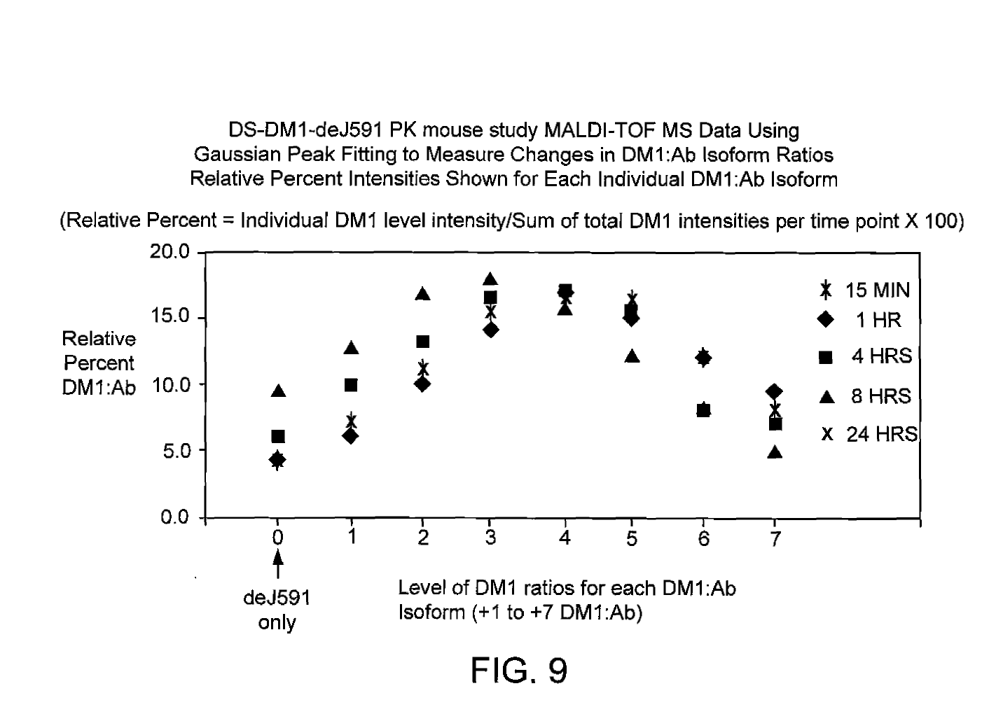

The UV-laser MALDI-TOF MS used in this study, as compared with IR-laser MALDI-TOF MS, has provided the ability to measure DOTA-deJ591 conjugates and determine the average level of DOTA coupling during conjugation reactions. To further examine details for these coupling profiles the MALDI-TOF MS mass spectra results may be processed and interpreted using Gaussian deconvolution and peak fitting software. As shown in FIG. 7, the doubly-protonated peak profile of DOTA-deJ591 is approximately twofold wider than that of the unconjugated deJ591. It was hypothesized that this resulted from a heterogeneous, partially-resolved, set of peaks representing a distribution of various DOTA:deJ591 conjugation levels. In order to determine the DOTA coupling distribution, the doubly-charged DOTA-deJ591 peak in FIG. 6 was produced using Gaussian deconvolution followed by Gaussian peak fitting. Comparing this processed peak result to the doubly-charged unconjugated deJ591 peak, in an overlay mass spectrum, provides the ability to identify the resolved and fitted peak for zero conjugation, with the adjacent peaks representing various degrees of DOTA conjugation. The results, consequently, display DOTA conjugation levels from zero DOTA up to seven DOTA, with an average conjugation level, based on total peak centroid measurement, of 4.9 DOTA. The resulting mass differences between the fitted peaks is an average of 518 mass units with a % CV of 3.2%, while the expected mass difference for each DOTA conjugation is 386 mass units. The expected mass addition of 386 was confirmed with previous measurements of DOTA-peptide conjugates using monoisotopic resolved mass assignment. The higher average mass value of 518 observed for each DOTA conjugation to the deJ591 antibody may be a result of the Gaussian deconvolution and peak fitting processes, where the software detects the underlying peaks resulting from multiple levels of DOTA conjugation, using the minimal representation and resolution present in the raw data. Another possibility for this higher average mass value is an unidentified contaminant forming an adduct ion with DOTA conjugated antibody, or DOTA molecules, when this intact antibody is prepared and analyzed using MALDI-TOF MS.

The conjugation level and distribution ratio were determined for DM1-deJ591 binding experiments. FIG. 7 shows the overlay mass spectrum view containing both the unconjugated deJ591 and DM1-deJ591 doubly-protonated peaks. As in the DOTA-deJ591 example, the zero level of DM1 incorporation can be determined by comparing the unconjugated deJ591 peak centroid with the first partially resolved peak from the conjugated DM1-deJ591 complex peak profile. The mass differences between adjacent peaks for the DM1-deJ591 complex resulted in an average mass of 826, with a % CV of 4.8, representing conjugation levels of zero DM1 up to seven DM1 molecules; this average mass difference is in good agreement with the expected mass of 852 for addition of a single DM1 molecule with the SPP linker. The observed accuracy for measuring DM1 conjugation, compared to the slightly less than expected mass differences observed for each measured DOTA-deJ591 conjugation level, may be a result of the improved resolution and peak definition obtained for the larger molecular weight DM1 molecules separating each DM1-deJ591 conjugation level.

Affinity purification using magnetic beads can be used to isolate and purify both unconjugated and conjugated antibody from a biological fluid, e.g. plasma or serum, for use with MALDI-TOF MS analysis. M270 Epoxy beads are suitable for this purpose. They are hydrophilic and have a slightly negative zeta-potential and have a diameter of 2.8 μm. Linker molecule is added to the beads according to the manufacturer's instructions (3 ug ligand (linker) per 10⁷ Dynal beads). A procedure for making and using antibody-coupled beads is provided below.
The beads are first washed as follows:
1 Weigh 15 mg beads (15 mg is about 10⁹ of beads) in an eppendorf tube.
2. Add 1 ml 0.1 M NaH2PO4, PH 7.4
3. Vortex for 30 seconds and incubate for 10 minutes with mixing.
4. Remove supernatant by placing the tube on a magnet and pipette off the liquid carefully.
5. Resuspend the beads in the same buffer, vertex and discard the supernatant in the same way as step 4.
This provides beads suitable for coating, which is performed as follows.
6. Add 1 ml of following:
   200 μl of 1 mg/ml anti-deJ591
   330 μl of 3 M ammonium sulfate (final is 1 M)
   470 μl of 0.1 M NaH2PO4
7. Mix thoroughly and incubate for 1 hour at room temperature.
8. Remove the supernatant and wash with PBS, 0.05% BSA 3 times.
9. Resuspend the beads with 1 ml PBS and store at 4° C. until use.
Target is isolated from plasma or serum as follows:
10. Dilute the plasma or serum in PBS.
11. Add 1 ml of diluted sample into the tube containing beads
12. Incubate 1 hour at room temperature.
13. Wash 2 times with PBS, 0.02% Tween-20, 2 times with PBS only
14. Elute the target with elution buffer (PH <2):
   Add 200 μl of elution buffer.
   Mix well and rotation 2 min.
   Repeat 2 more times.
   Total volume is 600 μl
The material is then concentrated with a Microcon (MW 100, Millipore)
15. Load the eluent onto the sample reservoir
16. Spin @ 6000 rpm for 6 min and discard the collect tube (all the flow through is in the tube).
17. Add 10 ul of 50:50 CAN:H2O into the sample reservoir, pipetting up and down to mix.
18. Change a new collect tube and spin @ 6000 rpm for 3 min.
19. The liquid in the collect tube is purified, concentrated sample and ready to use for MALDI analysis

### Example 7

### Metal element analysis of DOTA-NHS and DOTA-deJ591 by ICP/MS

Another important aspect of DOTA conjugated deJ591 is the amount of metal such as yttrium, ⁹⁰Y, which binds in the radiolabeling step. For optimum binding to occur trace metals must be minimized, since it has been shown that ⁹⁰Y-chelation can be affected by the concentration of trace metal contaminants. Several common elements, Fe, Pb and others, have stability constants that are similar to the stability constant of ⁹⁰Y binding to DOTA. Significant quantities of any of these elements could compromise the labeling reaction and lead to unacceptable radiopharmaceutical products. Trace metals are minimized by the use of metal-free containers when possible and chelex treated or Milli-Q Element A10 purified water. Despite these precautions, large amounts of trace metals may still exist in both DOTA-NHS and the conjugated deJ591 antibody. Knowing the amount of these elements before binding the radiolabeled metal to the conjugated DOTA-antibody complex is important in the manufacture of DOTA-conjugated antibodies developed as radiopharmaceutical products.

To test for the amounts of trace metals, ICP/MS was used to measure trace metals in biological samples. Trace analysis of the following elements ⁵⁶Fe, ⁵⁸Ni, ⁵⁹Co, ⁶³Cu, ⁶⁴Zn, ¹³⁹La, ¹⁴⁰Ce, and ²⁰⁸Pb was performed on both DOTA-NHS and DOTA conjugated deJ591 complexes. These elements were chosen based on their ability to bind to DOTA. Each of these trace metals should not exceed 100 ppb (ng/mL) or they will interfere with the labeling efficiency of ⁹⁰Y.

Briefly, DOTA-NHS lots were diluted in 1% nitric acid and infused on to the ICP/MS by passing through an ARIDUS (CETAC technologies, Omaha, Nebr.) desolvator. The temperatures for the membrane desolvator and spray chamber heater were 136° C. and 70° C., respectively. The sweep gas flow was set at 2 L/min and the nitrogen to 5 mL/min. The ICP/MS ran in full scan mode with the range being 5 to 250 amu. The intensities for the following elements ⁵⁶Fe, ⁵⁸Ni, ⁵⁹Co, ⁶³Cu, ⁶⁴Zn, ¹³⁹La, ¹⁴⁰Ce, and ²⁰⁸Pb were compared to a 10 ppb standard of ⁵⁹Co. The 1% nitric acid diluent was used for background subtraction. Using elemental targeting within the Data Explorer software (ABI, Framingham, Mass. 01701) matches were made for the isotope patterns for each of the desired elements. The deJ591-DOTA conjugate samples were diluted in 1% nitric acid to a concentration of 100 mg/mL. Samples were run in both selected ion recording (SIR) and full scan mode. The full scan mode used had a range of 5 to 250 amu. Masses monitored for SIR mode were ⁵⁶Fe, ⁵⁸Ni, ⁵⁹Co, ⁶³Cu, ⁶⁴ Zn, ¹³⁹La, ¹⁴⁰Ce, and ²⁰⁸Pb. Calibration curves for the standard mix were generated simultaneously for 100, 10, 1, 0.1, 0.01, and 0.001 ppb using Masslynx software (Micromass, UK).

Multielement analyses of two typical DOTA-NHS batches are shown in Table 18. The values were generated by comparing the intensities in the ICP/MS spectra for each element to the intensity for ⁵⁹Co. None of the elements show a concentration higher than 19 ppb and their combined total is less than 27 ppb for Batch A DOTA-NHS and 7 ppb for Batch B DOTA-NHS. Both batches have trace metal totals well below the criteria proposed for the elements. The next step was to analyze the trace metal content of the finished DOTA-deJ591 before labeling with ⁹⁰Y. For these experiments a standard mixture of the eight elements was used to generate simultaneous calibration curves. The intensities for each of the target elements in the ICP/MS spectrum were then compared with the individual calibration curves. The ICP/MS data for multielement analyses of the DOTA-deJ591 complex Batches deJ591 A and deJ591 B are shown in Table 19. The individual element values are consistently lower than the imposed cutoff of 100 ppb except for ⁶⁴Zn in Batch deJ591 B. However, the combined metal total for Batch deJ591 B is less than 178 ppb which is well below the 1 ppm (ug/mL) criteria for a total of the eight metals. Both DOTA-NHS and the DOTA conjugated deJ591 batches show sufficiently low amounts of the trace elements tested to ensure that ⁹⁰Y-chelation will not be affected by this aspect of the reaction.

TABLE 18
Table of Trace Metals Found in Two Batches of DOTA-NHS.

| | Batch A | Batch B |
|---|---|---|
| | DOTA-NHS | DOTA-NHS |
| Element | (ppb or ng/mL) | (ppb or ng/mL) |
| Fe | 4.00 | 5.00 |
| Ni | 0.09 | 0.15 |
| Co | 2.58 | 0.79 |
| Cu | 0.32 | 0.31 |
| Zn | 19.00 | 5.00 |
| La | ND | ND |
| Pb | 0.82 | 0.14 |
| Ce | ND | ND |

ND = Not Detected

TABLE 19
Table of trace metals found in two batches of DOTA conjugated deJ591.

| | Batch deJ591 | Batch deJ591 |
|---|---|---|
| Element | (ppb or ng/mL) | (ppb or ng/mL) |
| Fe | 21.61 | 12.78 |
| Ni | ND | 3.62 |
| Co | ND | 0.75 |
| Cu | 0.89 | 8.40 |
| Zn | 2.27 | 149.46 |
| La | 0.01 | 0.06 |
| Pb | 0.28 | 3.12 |
| Ce | ND | ND |

### Example 8

### Hisep RP-HPLC Analysis for Quantitation of DM1 Monomer, DM1 Dimer, DM1-TPA Adduct, PPA and Mercaptopyridine in DS-DM1-deJ591

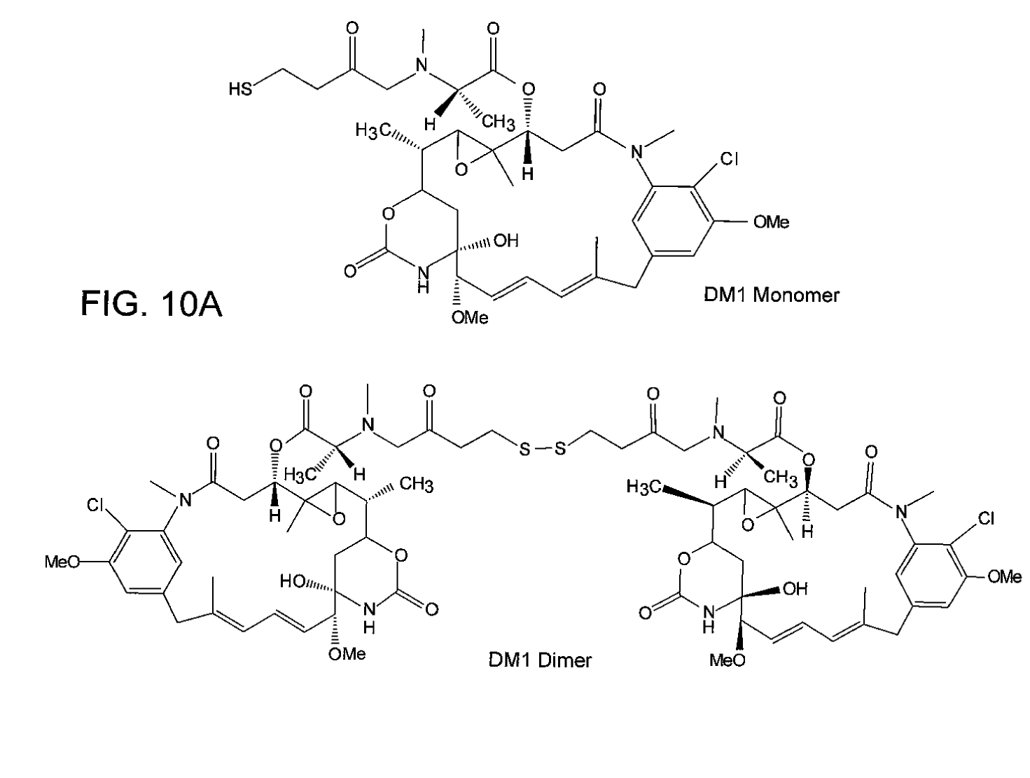

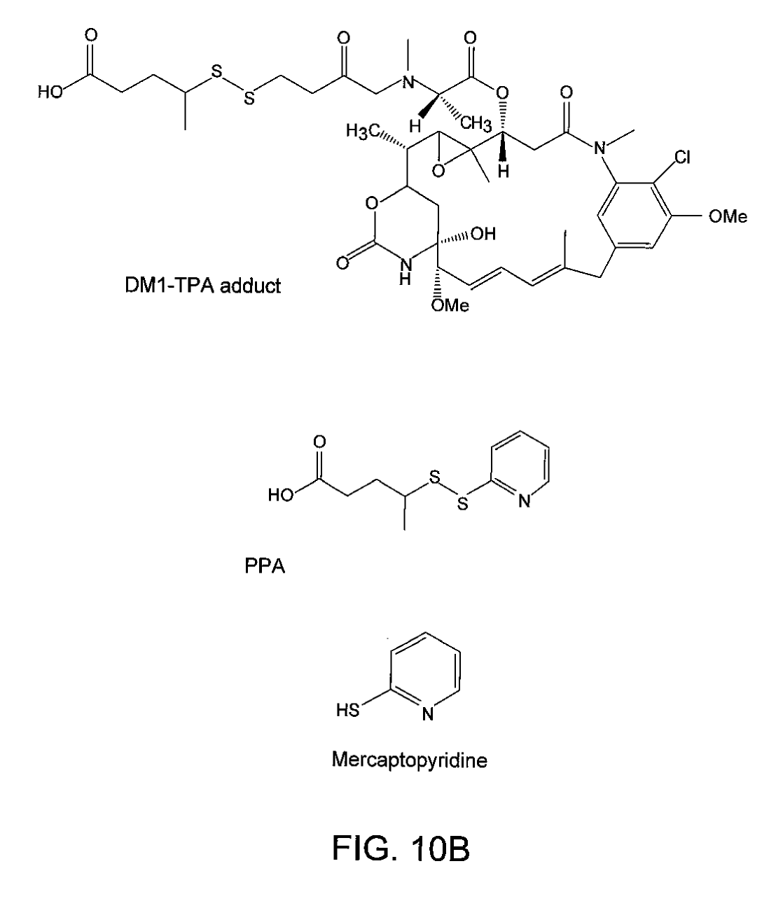

This Example describes the optimization and qualification of a reversed phase high performance liquid chromatography method used to quantitate the levels of DM1 monomer, DM1 dimer, DM1-TPA, PPA and mercaptopyridine in DS-DM1-deJ591. DS-DM1-deJ591 is an antibody/toxin conjugate consisting of DM1 conjugated to deJ591. Among the process-related and degradation impurities that are currently of concern are low molecular weight species structurally related to DM1, namely DM1 monomer, DM1 dimer, DM1-thiopentanoic acid adduct (DM1-TPA), 4-(2-pyridyldithio) pentanoic acid (PPA), and mercaptopyridine (see FIG. 10).

The maytansinoid DM1 is an analog of the well-characterized cytotoxic drug, maytansine. As such, the small molecular weight DM1-related impurities may present unwanted toxicity if present in DS-DM1-deJ591 drug product. In an effort to monitor and control the level of these DM1-related impurities, a reversed phase HPLC (RP-HPLC) method was developed to identify and quantitate the levels of such impurities in DS-DM1-deJ591. In the RP-HPLC method, a Hisep column (a C18-type reversed phase column manufactured by Supelco) was utilized because of its ability to elute large molecular weight species such as the intact antibody early, then subsequently separate small molecular weight species.

A method for the analysis of DM1 monomer, DM1 dimer, and DM1-TPA utilizing a 25% methanol solvent system for sample preparation was initially developed, but a stringent investigation of the methodology demonstrated that unacceptable recovery levels were obtained for the DM1 dimer species. In order to both improve the utility of the method for DM1 dimer quantitation and to extend its capabilities in terms of detecting other potential impurities, namely PPA and mercaptopyridine, the methodology was changed to a solvent system for sample preparation consisting of 50% acetonitrile/0.01% TFA. The subsequent modifications improved the performance of the method, and acceptable accuracy was observed with all five species of interest without impacting any other parameters. A crossover study was performed on the DM1 monomer and DM1-TPA species and demonstrated that initial data collected with the 25% methanol solvent system was still valid. In addition, studies were undertaken to determine the most appropriate formulation and storage conditions for the impurities standards.

### 8.1 Experimental Procedure

Reagents

For all chromatography, Mobile Phase A was 0.1% Trifluoroacetic acid (TFA) in water, and Mobile Phase B was 100% acetonitrile.

Tools/Equipment

Materials used included Trifluoroacetic acid (HPLC Grade, Pierce, Cat No., P128904); Acetonitrile (HPLC Grade, EMD Chemicals, Cat No., 75-05-8); Mercaptopyridine Standard (Sigma, Cat No., M5852); and Autosampler vials and caps (12×32 mm with pre-slit cap PTFE/Silicone septa, Waters, Cat No., WAT186000385).

Equipment used included a High Pressure Liquid Chromatography System consisting of a Waters 2695 Separations module with column heater unit, 2996 PDA detector and Millennium Data Chromatography Manager Software, Version 4.0; Hisep RP-HPLC Columns (25 cm×4.6 cm×5 μM, Supelco Cat No., 58919); and Hisep Guard Columns (4.0 mm×2 cm, 5 μm particle size, Supelco Cat No, 59640-U).

Procedure

Mobile Phase Blank was prepared by combining 300 μL of Mobile Phase A and 100 μL of Mobile Phase B.

A new Hisep RP-HPLC column and corresponding guard column were conditioned as follows. First, mobile phase (75% A: 25% B) was allowed to run through the column for a minimum of 8-12 hours or overnight at 0.1 mL/min. Prior to injection, the column was equilibrated at 1.0 ml/min for a minimum 1 hour. A minimum of 2 mobile phase blanks were injected to achieve a stable baseline. Once stable, test samples were injected onto the column.

For RP-HPLC analysis, the HPLC system was set up as follows. Mobile Phase A was 0.1% TFA/Water; Mobile Phase B was 100% acetonitrile; Flow Rate was 1.0 mL/minute; Column Temp. was 25° C. (±2° C.); Sample Temp was 4° C. (±3° C.); Injection Volume was 50.0 μL; Output Wavelengths were 252 and 280 nm; Run Time was 31.0 minutes (Analysis window 18.0 minutes); and Sampling Rate was 1.0 pts/sec. The gradient conditions are set out in Table 20. Samples were arranged into the autosampler according to Table 21.

TABLE 20
Gradient Conditions

| | Time | Flow | | | |
|---|---|---|---|---|---|
| | (min) | (mL/min) | % A | % B | Curve |
| 1 | — | 1.00 | 75.0 | 25.0 | — |
| 2 | 5.00 | 1.00 | 75.0 | 25.0 | 6 |
| 3 | 15.00 | 1.00 | 65.0 | 35.0 | 6 |
| 4 | 20.00 | 1.00 | 10.0 | 90.0 | 6 |
| 5 | 20.50 | 1.00 | 75.0 | 25.0 | 6 |
| 6 | 30.00 | 1.00 | 75.0 | 25.0 | 6 |
| 7 | 31.00 | 1.00 | 75.0 | 25.0 | 6 |

TABLE 21
Typical Injection Sequence for Three DS-DM1-deJ591 Test Samples

| | | |
|---|---|---|
| Sample | No. | Amt. Injected |

| No. | Sample Name | of Injections | μM | pmol |
|---|---|---|---|---|
| 1 | Blank (Mobile | 1 | — | — |
| 2 | Blank (Diluent) | 1 | — | — |
| 6 | DM1 Std. | 2 | 5.00 | 250.0 |
| 7 | DM1-Dimer | 2 | 5.00 | 250.0 |
| 8 | DM1-TPA Std. | 2 | 5.00 | 250.0 |
| 9 | M-pyd Std. | 2 | 1.25 | 62.5 |
| 10 | PPA Std. | 2 | 2.50 | 125.0 |
| 11 | Blank (Diluent) | 1 | N/A | N/A |
| 12 | Test Sample-1 | 2 | TBD | TBD |
| 13 | Test Sample-2 | 2 | TBD | TBD |
| 14 | Test Sample-3 | 2 | TBD | TBD |
| 15 | Blank (Diluent) | 1 | — | — |

The following system suitability criteria were followed. There should be no spurious peaks in the diluent blank (from 3.5 to 18 minutes) from both 280 and 252 nm that interfere with the sample analysis. The peak area of all 100% standards should pass the specification as described in Table 22.

TABLE 22
Peak Area and LOQ values for the Standards

| | | | |
|---|---|---|---|
| Standard | Standard | LOQ | Minimum |

| No. | Name | μM | pmol | Area of 100% Std |
|---|---|---|---|---|
| 1 | DM1 | 0.125 | 6.25 | 300000 |
| 2 | DM1- | 0.250 | 12.5 | 750000 |
| 3 | DM1- | 0.062 | 3.1 | 350000 |
| 4 | M-pyd | 0.150 | 7.5 | 20000 |
| 5 | PPA Std | 0.312 | 15.6 | 13000 |

The data were analyzed as follows. Average peak area was calculated for each of the standards (DM1, DM1-Dimer, DM1-TPA, M-pyd and PPA) from the replicate injections. From the average peak area of the standards the area/pmol was calculated. The average peak area of the components found in the test samples was calculated. The amount (pmol) of each component in the test samples was calculated by dividing the average area of each component by the area/pmol from the corresponding standard.

If a particular component in the test sample was not detected, the value was reported as “<X pmol”, where X=LOQ of the standard in pmol (see Table 22).

The pmol obtained from the above components for the specified injection volume was converted into pmol/L as follows:

$$pmol \text{/} L = \frac{amt . pmol \times 1000}{inj . vol . ( ml )}$$

The pmol/L value from the above components was converted into μM as follows:

$$μM = \frac{pmol \text{/} L value}{1 \text{,} 000 \text{,} 000}$$

The pmol/mg of DS-DM1-deJ591 was calculated as follows:

$$pmol \text{/} mg = \frac{pmol \text{/} L value}{protein conc . ( mg \text{/} mL ) \times 1000} .$$

The Theoretical pmol DM1/mg protein was calculated as follows:

$$Theoretical pmol DM 1 / mg protein = \frac{DM 1 / Ab Ratio \times 1}{0.000147224}$$

The % Free DM1 Species was calculated as follows:

$$\% Free DM 1 Species = \frac{pmol \text{/} mg protein value \times 100}{theoretical pmol DM 1 / mg protein value} .$$

Two distinct methods were used in this Example. The original method was developed for the analysis and quantitation of DM1 monomer, DM1 dimer, and DM1-TPA. Samples were prepared in 25% methanol and separated on a water/acetonitrile/TFA gradient, with detection at 254 nm. The method was modified to yield better accuracy for the DM1 dimer, and extended to use with PPA and mercaptopyridine. The modified method utilized the same water/acetonitrile/TFA gradient, with samples prepared for injection using 50% acetonitrile/0.01% TFA. Separation was monitored at 252 nm and 280 nm. Qualified standards were prepared and a crossover study was carried out to compare the qualification work performed using the original method with the improved method.

Specificity (Selectivity)

To identify the retention times of DM1 monomer, DM1 dimer, DM1-TPA adduct, PPA, and mercaptopyridine, individual solutions at the working concentrations of each of these DM1-related impurities were analyzed. For the purposes of this Example, the working concentrations of the DM1 monomer, DM1 dimer, and DM1-TPA adduct are defined as 5 μM, and for PPA and mercaptopyridine as 2.5 μM and 1.25 μM, respectively. A solution consisting of a mixture of each of the five compounds was also injected at the individual working concentrations to demonstrate resolution of the species. Additionally, peak purity analysis was performed to demonstrate that the resulting peaks are spectrally homogeneous.

Sample Stability

A solution containing working concentrations of DM1 monomer, DM1 dimer, DM1-TPA adduct, PPA, and mercaptopyridine in DS-DM1-deJ591 was prepared and stored in the autosampler for a period of 24 hours at ambient temperature. The sample was analyzed at times t=0 h, 12 h, and 24 h. Sample stability was determined by comparing the area percent (area %) values of the stored sample to that of the initially-prepared solution (t=0 h).

Precision

Instrument precision was assessed for both the standard solutions of DM1-related impurities and for standards in the presence of DS-DM1-deJ591. Instrument precision was assessed by analyzing six replicates each from single solutions of DM1 monomer, DM1 dimer, DM1-TPA adduct, PPA, mercaptopyridine alone and in the presence of DS-DM1-deJ591 at 100% of standard injection amounts and determining the percent relative standard deviations (% RSD).

Repeatability was assessed by analyzing triplicates of DM1 monomer, DM1 dimer, DM1-TPA, PPA, and mercaptopyridine prepared at 80, 100, and 120% of the working concentrations in 50% acetonitrile/0.01% TFA. Samples were analyzed by two different analysts using two different instruments and columns. The percent relative standard deviations (% RSD) were then determined.

Intermediate precision was assessed by using the data generated by two different analysts on two different days. The data generated from the intra-assay precisions were used to calculate the inter-assay precision.

Standard Linearity

Linearity of the method was evaluated across the range consisting of 5%-200% of the working concentrations of DM1 monomer, DM1 dimer and DM1-TPA adduct (0.25-10 μM). For PPA and mercaptopyridine, linearity over the range of 6%-400% was evaluated (0.15-10 μM and 0.075-5 μM, respectively). All standard solutions were prepared by dilution of a stock solution for each of the DM1-related impurities, and each solution was analyzed in triplicate.

The linear relationship of the above samples were evaluated by first-order least squares linear regression analysis. The correlation coefficient (r²), y-intercept, and slope of the regression line were calculated.

Spiking Recovery (Accuracy) and Linearity

The linearity of the area responses of the DM1-related impurities in the presence of DS-DM1-deJ591 were evaluated across the range of 5-200% of the working concentrations of DM1 monomer, DM1 dimer, and DM1-TPA adduct, and across the range of 6-400% of the working concentration of PPA and mercaptopyridine. The linear relationship of the above samples were evaluated by first-order least squares linear regression analysis. The correlation coefficient (r²), y-intercept and slope of the regression line were calculated.

In order to more carefully evaluate the performance of the method with DM1 dimer, an additional study was performed by a second analyst. In this set of experiments, triplicates of DM1 dimer at concentrations of 80, 100, and 120% of the working concentration were spiked into DS-DM1-deJ591 and the percent recovery was determined.

Limit of Detection (LOD)

The LOD was determined by the analysis of different concentrations of samples and establishing the minimum level at which the signal-to-noise ratio (S/N) was ideally ≧3. S/N ratios were determined using the peak-to-peak noise method calculated by the Millennium 3.20 Data Chromatography Manager Software. Noise value in each chromatogram is determined over a time of approximately two minutes. The signal was taken from the relative height of the desired peak in the chromatogram. For this purpose, triplicate injections with concentrations ranging 10-0.075 μM for the DM1 monomer, DM1 dimer and DM1-TPA, 40-0.077 μM for PPA and 5-0.039 μM for mercaptopyridine were made. The data obtained from the linearity studies was used for estimating the LOD of the method.

Limit of Quantitation (LOQ)

The LOQ was established by the analysis of the same sample replicates used in determining the LOD. As described above, S/N ratios were calculated using the peak-to-peak noise method. Specifically, the LOQ was determined by selecting the concentration that achieves a S/N ratio that was ideally ≧10.

Range

The range of the method was determined from the data obtained from the linearity studies. The upper and lower limits of the method were defined as the highest and lowest concentrations where acceptable linearity was demonstrated for the method.

### 8.2 Results and Discussion

Specificity (Selectivity)

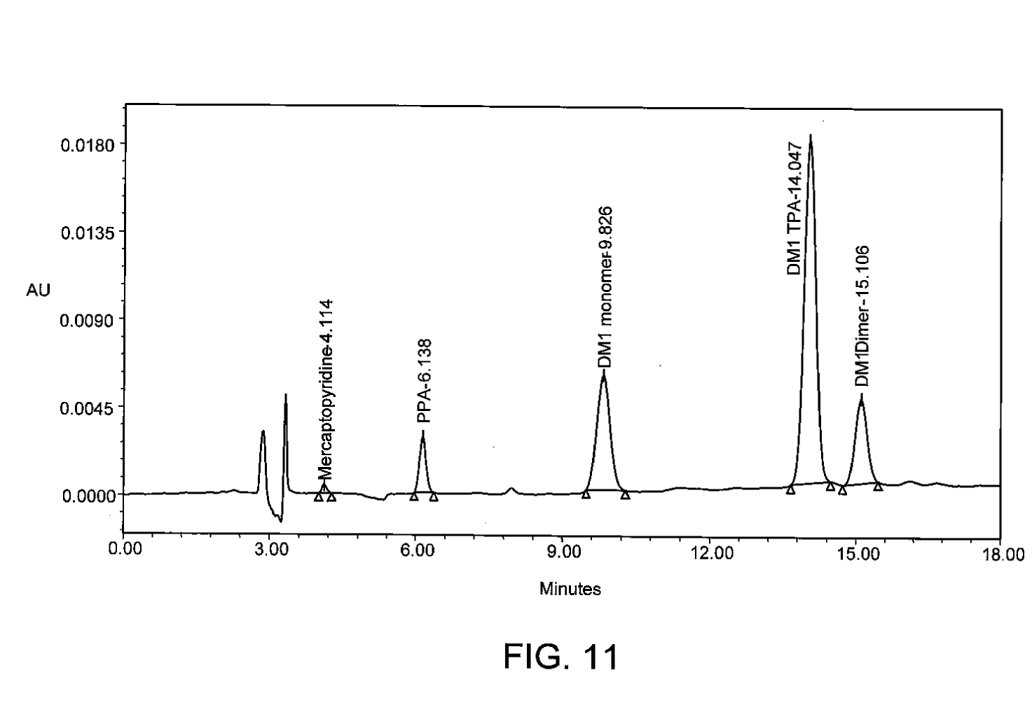

The specificity results indicate that there is no significant interference between the DM1 monomer, DM1 dimer, DM1-TPA adduct, PPA, and mercaptopyridine. As shown in FIG. 11, the peaks for each of the species at their working concentrations were resolved from one other, with retention times of approximately 4.1 minutes (mercaptopyridine), 6.1 minutes (PPA), 9.8 minutes (DM1 monomer), 14.1 minutes (DM1-TPA adduct) and 15.1 minutes (DM1 dimer).

Peak purity assessments of the DM1 monomer, DM1 dimer, DM1-TPA adduct, PPA, and mercaptopyridine are summarized in Table 23. A peak is considered to be spectrally pure if the calculated purity angle value is less than specified purity threshold angle value. As shown in Table 23, this was true for all five DM1-related impurities, indicating peak homogeneity across the whole peak for the DM1 monomer, DM1 dimer, DM1-TPA adduct, PPA, and mercaptopyridine.

Since the five DM1-related impurities were resolved from each other and the purity angles of these peaks were lower than the purity threshold angles, the DM1-related species peaks were presumed to be homogeneous and adequately resolved from any close eluting impurity under the analysis conditions. Therefore, based on the data, the method was specific for the measurement of DM1 monomer, DM1 dimer, DM1-TPA, PPA, and mercaptopyridine in DS-DM1-deJ591 samples.

TABLE 23
Peak Purity Analysis: Purity Angles and Purity Threshold Angles for
DM1 Monomer, DM1 Dimer, DM1-TPA Adduct, PPA, and
Mercaptopyridine

| | | Purity Threshold |
|---|---|---|
| Compound | Purity Angle | Angle |
| DM1 monomer | 0.350 | 0.602 |
| DM1 dimer | 0.655 | 1.150 |
| DM1-TPA adduct | 0.194 | 0.375 |
| PPA | 0.694 | 1.701 |
| Mercaptopyridine | 2.047 | 2.844 |

Preparation of Impurities Standards

Individual qualified standard stocks of three of the five species of interest (DM1 monomer, DM1 dimer, and DM1-TPA) were prepared. PPA and mercaptopyridine stocks were prepared at concentrations of 10 μM and 5 μM, respectively. In brief, all stocks were prepared in a solution of 50% acetonitrile/0.01% TFA, dispensed in 100 μL aliquots and stored at −80±10° C. Prior to use, stock aliquots were diluted 1:4 in 50% acetonitrile/0.01% TFA. The DM1 dimer, mercaptopyridine, and PPA stocks prepared in this manner were used for the entirety of this Example. The DM1 monomer and DM1-TPA adduct stocks were used for the crossover study described below and correlated to work performed with earlier stock solutions prepared in 25% methanol.

However, due to problems with long-term stability of stock solutions prepared as described above, other potential methods for preparation/storage were examined. After a period of approximately eight months, inconsistencies in the observed peak areas were seen relative to historical data. Potential causes for the problem were slow evaporation of solvent magnified by the small initial starting volume and/or difficulties in redissolving the standard after thawing. Two solutions examined to alleviate the standard stability issue were to either use a significantly more dilute initial concentration or to lyophilize the material after creating aliquots of known amounts.

Four vials each of DM1 monomer, DM1 dimer, and DM1-TPA were lyophilized individually. All vials contained a total of 15 nmol. Two vials of each standard were reconstituted by adding 150 μL of 50% acetonitrile/0.01% TFA to make a 100 μM stock solution; the remaining vials were reconstituted with 300 μL to make 50 μM stocks. The stocks were further diluted to the working concentration of 5 μM and analyzed. As shown in Table 24, lyophilization is not an appropriate technique to use for the DM1 monomor, as significant DM1 monomer→dimer conversion was observed. Preparation of more dilute standard stock solutions appeared to be a viable solution. The stability of these standards were monitored over time.

TABLE 24
Comparison of 100 μM and 50 μM Lyophilized DM1 Monomer Stocks

| | | | | | 100 μM |
|---|---|---|---|---|---|
| | 50 μM stock - | | 100 μM stock - | | stock - |
| | lyophilized | | lyophilized | | untreated |
| | Monomer | | Monomer | | Monomer |
| | area | Dimer area | area | Dimer area | area |
| Sample | observed | observed | observed | observed | observed |
| Vial 1: | | | | | |
| 1a | 476970 | N/A | 392043 | 10508 | 459407 |
| 1b | 475075 | N/A | 391526 | 8635 | 460238 |
| 1c | 476660 | N/A | 389675 | 11114 | 459885 |
| Average | 476235 | N/A | 391081 | 10086 | 459843 |
| SD | 1016 | N/A | 1245 | 1292.337 | 417 |
| % RSD | 0.2 | N/A | 0.3 | 12.814 | 0.1 |
| Vial 2: | | | | | |
| 2a | 415292 | 10097 | 418902 | 10029 | NT |
| 2b | 411974 | 10809 | 418204 | 10666 | NT |
| 2c | 410632 | 11100 | 415463 | 9780 | NT |
| Average | 412633 | 10669 | 417523 | 10158 | N/A |
| SD | 2399 | 516.016 | 1818 | 456.940 | N/A |
| % RSD | 0.6 | 4.837 | 0.4 | 4.498 | N/A |

N/A = not applicable
NT = not tested

Effect of Solvent for Sample Preparation

Initial experiments examining DM1 monomer, DM1 dimer, and DM1-TPA in the Hisep method were performed using a solvent system for sample preparation consisting of 25% methanol. The resulting data indicated that the accuracy of recovery of the monomer and DM1-TPA was good (94.60% and 97.61%, respectively) but was perhaps unacceptable for the DM1 dimer (37.85%). Based upon this information, a study to examine the effect of extraction solvent was performed. In conjunction with these experiments, the extraction efficiency of the two other potential impurities were included, PPA and mercaptopyridine.

The results of the initial solvent extraction experiments indicated that the best sample preparation condition for the DM1 dimer was 50% acetonitrile/0.01% TFA (see Table 25). Additional acetonitrile yielded a hazy solution and a poor separation profile; 75% methanol/0.01% TFA gave comparable results to the 50% acetonitrile/0.01% TFA but was less desirable to work with. Based upon these results, the sample preparation procedure was modified to use 50% acetonitrile/0.01% TFA.

TABLE 25
Effect of Extraction Solvent on Recovery of DM1 Dimer Using 5 μM
Working Solution

| | Spiked area | Unspiked area | % |
|---|---|---|---|
| | (n = 3) | (n = 3) | Recovery |
| Methanol | | | |
| concentration | | | |
| 25% | n/a | 375762 | n/a |
| 50% | 561705 | 746516 | 75.2 |
| 75% | 810984 | 839007 | 96.7 |
| Acetonitrile | | | |
| 25% | n/a | n/a | n/a |
| 50% | 755096 | 807749 | 93.48 |
| 75% | n/a | n/a | n/a |

Standards Crossover Study

In order to demonstrate that earlier data obtained for the DM1 monomer and DM1-TPA in experiments carried out using the 25% methanol extraction system did not impact the results of these studies, a crossover study comparing extraction efficiency for both of these species was performed. In addition, a comparison was made between the two sets of standards used for the DM1 monomer and DM1-TPA.

The extraction crossover results are presented in Table 26. The data show that the efficiency of extraction for both the DM1 monomer and the DM1-TPA using either the 25% methanol system or the 50% ACN/0.01% TFA was comparable, indicating that this change in the methodology would have not affected data collected using the 25% methanol extraction protocol in use at an earlier date.

Table 27 summarizes the comparison of the two standards prepared for the DM1 monomer, DM1 dimer, and the DM1-TPA adduct. The results of this comparison indicate that there is little difference in any of their concentrations, so that earlier work performed with the older stocks was still valid.

TABLE 26
Comparison of the Efficiency of Extraction of DM1 Monomer and
DM1-TPA Adduct Using 25% Methanol and 50% Acetonitrile/0.01%
TFA

| Concentration | DM1 | DM1 | DM1-TPA | DM1-TPA |
|---|---|---|---|---|
| level/ | 25% | 50% ACN/ | 25% | 50% ACN/0.01% |
| Replicate No. | Methanol | 0.01% TFA | Methanol | TFA |
| 80% | | | | |
| Rep. #1 | 287180 | 271531 | 311051 | 322382 |
| Rep. #2 | 288714 | 251038 | 311115 | 333869 |
| Rep. #3 | 288461 | 264906 | 312630 | 358890 |
| Average = | 288118 | 262492 | 311599 | 338380 |
| SD = | 822 | 10458 | 894 | 18667 |
| % RSD = | 0.3 | 4.0 | 0.3 | 5.5 |
| 100% | | | | |
| Rep. #1 | 346079 | 339524 | 387491 | 387016 |
| Rep. #2 | 352347 | 320596 | 385933 | 388947 |
| Rep. #3 | 356079 | 349506 | 384500 | 388751 |
| Average = | 351502 | 336542 | 385975 | 388238 |
| SD = | 5053 | 14684 | 1496 | 1063 |
| % RSD = | 1.4 | 4.4 | 0.4 | 0.3 |
| 120% | | | | |
| Rep. #1 | 437121 | 421097 | 507649 | 503964 |
| Rep. #2 | 440968 | 432519 | 510492 | 514745 |
| Rep. #3 | 440968 | 440981 | 537270 | 550333 |
| Average = | 439686 | 431532 | 518470 | 523014 |
| SD = | 2221 | 9979 | 16343 | 24265 |
| % RSD = | 0.5 | 2.3 | 3.2 | 4.6 |

TABLE 27
Comparison of the Two Stock Standards of DM1 Monomer, DM1
Dimer, and DM1-TPA

| | | Original standard | | | New standard | | | |
|---|---|---|---|---|---|---|---|---|
| | Concentration | Injection | Injection | | Injection | Injection | | % |
| Sample | (μM) | #1 | #2 | Avg | #1 | #2 | Avg | diff |
| DM1 | 0.25 | 7449 | 8478 | 7963.5 | 7368 | 7276 | 7322 | 8.3 |
| monomer | 0.5 | 19627 | 19399 | 19513 | 20538 | 19616 | 20077 | 1.7 |
| | 1.0 | 39984 | 40048 | 40016 | 52407 | 52125 | 52266 | 30.4 |
| | 2.5 | 109868 | 110274 | 110071 | 122225 | 123769 | 122997 | 12.1 |
| | 5.0 | 223654 | 223627 | 223640 | 255949 | 254849 | 255399 | 14.1 |
| | 7.5 | 335428 | 336568 | 335998 | 382624 | 382812 | 382718 | 13.9 |
| | 10.0 | 461364 | 462114 | 461739 | 521439 | 518253 | 519846 | 12.4 |
| DM1 | 0.25 | 10965 | 11557 | 11261 | 10546 | 11701 | 11124 | 1.2 |
| dimer | 0.5 | 19654 | 24953 | 22304 | 18538 | 24498 | 21518 | 3.5 |
| | 1.0 | 49028 | 54795 | 51911.5 | 59255 | 66850 | 63053 | 21.5 |
| | 2.5 | 161484 | 173522 | 167503 | 167175 | 191828 | 179502 | 7.2 |
| | 5.0 | 361497 | 381222 | 371360 | 372337 | 389111 | 380724 | 2.5 |
| | 7.5 | 528588 | 562835 | 545712 | 559936 | 595406 | 577671 | 5.9 |
| | 10.0 | 774806 | 832657 | 803732 | 818575 | 888608 | 853592 | 6.2 |
| DM1- | 0.25 | 15948 | 15636 | 15792 | 24727 | 24702 | 24715 | 56.5 |
| TPA | 0.5 | 31950 | 32909 | 32430 | 43612 | 43322 | 43467 | 34.0 |
| adduct | 1.0 | 66361 | 67051 | 66706 | 90061 | 90246 | 90154 | 35.2 |
| | 2.5 | 177263 | 177861 | 177562 | 206274 | 206405 | 206340 | 16.2 |
| | 5.0 | 335553 | 335542 | 335548 | 376957 | 378978 | 377968 | 12.6 |
| | 7.5 | 505316 | 505925 | 505621 | 589906 | 594742 | 592324 | 17.1 |
| | 10.0 | 689531 | 690603 | 690067 | 785497 | 789393 | 787445 | 14.1 |

Sample Stability

The sample stabilities of DM1 monomer, DM1 dimer, PPA, and mercaptopyridine in the presence of DS-DM1-deJ591 were determined by analyzing solutions of DS-DM1-deJ591 spiked with working concentrations of each of these analytes individually at t=0, 12, and 24 hours. The DM1-TPA adduct was not spiked into the solution due to the already high level of this species in DS-DM1-deJ591, but was analyzed directly.

Satisfactory stability was observed for DM1 monomer, DM1 dimer, DM1-TPA adduct, PPA and mercaptopyridine, with greater than 95% of the analytes remaining after 24 hours (see Table 28).

TABLE 28
Sample Stability Data of DM1 Monomer, DM1 Dimer, DM1-TPA
Adduct, PPA, and Mercaptopyridine with DS-DM1-deJ591 in the
Autosampler

| | Average peak area | | |
|---|---|---|---|
| compound | t = 0 h | t = 12 h^{a} | t = 24 h^{a} |
| DM1 monomer | 223667 | 217293 (97.15%) | 214265 (95.79%) |
| DM1 dimer | 759441 | 756467 (99.61%) | 752857 (99.13%) |
| DM1-TPA adduct | 246302 | 245410 (9.63%) | 245126 (99.52%) |
| PPA | 15881 | 15377 (96.83%) | 15595 (98.20%) |
| Mercaptopyridine | 15446 | 15654 (101.35%) | 15620 (101.13%) |

^{a}Values in parenthesis represent the % remaining area counts at a given time point relative to the t = 0 h time point.

Precision

Six replicates of the standard solutions of DM1 monomer, DM1 dimer, DM1-TPA, PPA, and mercaptopyridine were separately analyzed and the results are summarized in Table 29. The % RSD was determined to be 0.24, 0.015, 0.27, 1.53, and 0.94% for the DM1 monomer, DM1 dimer, DM1-TPA adduct, PPA, and mercaptopyridine, respectively.

Six replicates of impurities in the presence of DS-DM1-deJ591 were also analyzed and the results are given in Table 30. In order to maximize comparability between instrument precision results obtained with standard solutions and DS-DM1-deJ591, DS-DM1-deJ591 spiked with a solution of 100% working concentration of DM1 monomer, DM1 dimer, PPA, and mercaptopyridine was analyzed in place of neat DS-DM1-deJ591. The amount of DM1-TPA present in the DS-DM1-deJ591 sample did not necessitate further spiking for this study. The % RSD was determined to be 1.01, 0.59, 0.82, 1.22, and 0.78% for the DM1 monomer, DM1 dimer, DM1-TPA adduct, PPA, and mercaptopyridine, respectively.

The results from the standard solutions and spiked DS-DM1-deJ591 solutions indicate that the RP-HPLC method was performing with suitable instrument precision.

TABLE 29
Instrument Precision: DM1 Monomer, DM1 Dimer, DM1-TPA
Adduct, PPA, and Mercaptopyridine in Standard Solutions

| Replicate | DM1 monomer | DM1 dimer | DM1-TPA | PPA | Mercaptopyridine |
|---|---|---|---|---|---|
| No. | (area) | (area) | adduct (area) | (area) | (area) |
| 1 | 218813 | 463573 | 278860 | 15450 | 13389 |
| 2 | 218967 | 462492 | 277920 | 15987 | 13336 |
| 3 | 217947 | 459287 | 277436 | 15501 | 13582 |
| 4 | 219171 | 460437 | 278731 | 15319 | 13655 |
| 5 | 218985 | 449400 | 276990 | 15746 | 13444 |
| 6 | 218006 | 447245 | 277517 | 15633 | 13583 |
| Average = | 218648 | 457072 | 277909 | 15606 | 13498 |
| SD = | 533 | 6975 | 749 | 238 | 126 |
| % RSD = | 0.24 | 0.015 | 0.27 | 1.53 | 0.94 |

TABLE 30
Instrument Precision: DM1 Monomer, DM1 Dimer, DM1-TPA
Adduct, PPA, and Mercaptopyridine in DS-DM1-deJ591

| Replicate | DM1 monomer | DM1 dimer | DM1-TPA | PPA | Mercaptopyridine |
|---|---|---|---|---|---|
| No. | (area) | (area) | adduct (area) | (area) | (area) |
| 1 | 226590 | 756767 | 300961 | 14262 | 15053 |
| 2 | 226897 | 762144 | 301986 | 14123 | 14982 |
| 3 | 230120 | 757575 | 302740 | 14294 | 14884 |
| 4 | 229791 | 753698 | 304692 | 14078 | 15058 |
| 5 | 224178 | 751248 | 299841 | 14120 | 15218 |
| 6 | 225818 | 750049 | 297485 | 13808 | 15146 |
| Average = | 227232 | 755247 | 301284 | 14114 | 15057 |
| SD = | 2313 | 4488 | 2484 | 173 | 118 |
| % RSD = | 1.01 | 0.59 | 0.82 | 1.22 | 0.78 |

Intra-Assay Precision (Repeatability)

Triplicate sample preparations of each of the five species at 80, 100, and 120% of the working concentration of DS-DM1-deJ591 were analyzed by the same analyst and the results are shown in Table 31. The % RSD values at the three concentrations analyzed for each species were determined to be <4.5, <0.5, <1.2, <3.4, and <3.1 for the DM1 monomer, DM1 dimer, DM1-TPA, PPA, and mercaptopyridine, respectively. The results indicate that the method was performing with suitable repeatability.

A second set of data generated by a second analyst is summarized in Table 32. % RSD values were <0.7 (DM1 monomer), <0.7 (DM1 dimer), <1.3 (DM1-TPA), <9.5 (PPA), and <1.6 (mercaptopyridine). Good repeatability was also observed in this set of experiments, confirming the suitability of the method.

TABLE 31
Intra-Assay Precision (Repeatability) - Analyst 1

| Concentration level/ | DM1 monomer | DM1 dimer | DM1-TPA | PPA | Mercaptopyridine |
|---|---|---|---|---|---|
| Replicate No. | (252 nm) | (252 nm) | (252 nm) | (252 nm) | (280 nm) |
| 80% | | | | | |
| Rep. #1 | 271531 | 724668 | 297716 | 11549 | 21199 |
| Rep. #2 | 251038 | 729452 | 298350 | 11582 | 21343 |
| Rep. #3 | 264906 | 730754 | 296879 | 11737 | 21263 |
| Average = | 262492 | 728291 | 297648 | 11623 | 28114 |
| SD = | 10458 | 3205 | 738 | 100 | 838 |
| % RSD = | 4.0 | 0.4 | 0.2 | 0.9 | 3.0 |
| 100% | | | | | |
| Rep. #1 | 339524 | 884027 | 389531 | 16045 | 26290 |
| Rep. #2 | 320596 | 887592 | 389156 | 15241 | 26838 |
| Rep. #3 | 349506 | 885744 | 390760 | 15108 | 26791 |
| Average = | 336542 | 885788 | 389816 | 15465 | 26640 |
| SD = | 14684 | 1783 | 839 | 507 | 304 |
| % RSD = | 4.4 | 0.2 | 0.2 | 3.3 | 1.1 |
| 120% | | | | | |
| Rep. #1 | 421097 | 1064456 | 443960 | 19495 | 32209 |
| Rep. #2 | 432519 | 1061758 | 442264 | 18830 | 32265 |
| Rep. #3 | 440981 | 1068369 | 442789 | 18742 | 32963 |
| Average = | 431532 | 1064861 | 443004 | 19022 | 32479 |
| SD = | 9979 | 3324 | 868 | 412 | 420 |
| % RSD = | 2.3 | 0.3 | 1.1 | 2.2 | 1.3 |

TABLE 32
Intra-Assay Precision (Repeatability) - Analyst 2

| Concentration level/ | DM1 Monomer | DM1 Dimer | DM1-TPA | PPA | Mercapto-pyridine |
|---|---|---|---|---|---|
| Replicate No. | (252 nm) | (252 nm) | (252 nm) | (252 nm) | (280 nm) |
| 80% | | | | | |
| Rep. #1 | 214054 | 604921 | 342390 | 17878 | 24965 |
| Rep. #2 | 216693 | 605244 | 341465 | 17234 | 24657 |
| Rep. #3 | 215598 | 611875 | 340915 | 18397 | 24208 |
| Average = | 215448 | 607347 | 341590 | 17836 | 24610 |
| SD = | 1326 | 3925 | 745 | 583 | 381 |
| % RSD = | 0.6 | 0.6 | 0.2 | 3.3 | 1.5 |
| 100% | | | | | |
| Rep. #1 | 267578 | 807102 | 425810 | 20392 | 28693 |
| Rep. #2 | 270766 | 810364 | 429388 | 22250 | 28236 |
| Rep. #3 | 268518 | 805780 | 436152 | 24603 | 28289 |
| Average = | 268954 | 807749 | 430450 | 22415 | 28406 |
| SD = | 1638 | 2359 | 5252 | 2110 | 250 |
| % RSD = | 0.6 | 0.3 | 1.2 | 9.4 | 0.9 |
| 120% | | | | | |
| Rep. #1 | 321374 | 960641 | 516591 | 27030 | 33373 |
| Rep. #2 | 321202 | 968340 | 525659 | 27074 | 33216 |
| Rep. #3 | 319804 | 963105 | 526749 | 27710 | 33820 |
| Average = | 320793 | 964029 | 523000 | 27271 | 33470 |
| SD = | 861 | 3932 | 5577 | 381 | 313 |
| % RSD = | 0.3 | 0.4 | 1.1 | 1.4 | 0.9 |

Inter-Assay Precision

The inter-assay precision was calculated from the intra-assay precision data obtained from two analysts on two different days. The results are summarized in Table 33. % RSD values of <10% was observed for DM1 dimer, DM1-TPA and mercaptopyridine. Slightly higher values, <17% and <24%, were observed for DM1 monomer and PPA, respectively. Although tight % RSD values were observed during intra-assay precision for all the standards by each analyst, there was a slight increase in the % RSD values, particularly for DM1 monomer and PPA, for the inter-assay precision. This was mainly due to the high concentration of the standard stock solutions that was used for dilution to generate the working standards. Further studies with about 100 mM stock solutions gave very good inter-assay precision (data not shown).

TABLE 33
Inter-Assay Precision

| Concentration level/ | DM1 monomer | DM1 dimer | DM1-TPA | PPA | Mercaptopyridine |
|---|---|---|---|---|---|
| Replicate No. | (252 nm) | (252 nm) | (252 nm) | (252 nm) | (280 nm) |
| 80% | | | | | |
| Rep. #1 | 214054 | 604921 | 342390 | 17878 | 24965 |
| Rep. #2 | 216693 | 605244 | 341465 | 17234 | 24657 |
| Rep. #3 | 215598 | 611875 | 340915 | 18397 | 24208 |
| Rep. #4 | 271531 | 724668 | 297716 | 11549 | 21199 |
| Rep. #5 | 251038 | 729452 | 298350 | 11582 | 21343 |
| Rep. #6 | 264906 | 730754 | 296879 | 11737 | 21263 |
| Average = | 238970 | 667819 | 319619 | 14730 | 22939 |
| SD = | 26615 | 66322 | 24077 | 3424 | 1847 |
| % RSD = | 11.1 | 9.9 | 7.5 | 23.2 | 8.1 |
| 100% | | | | | |
| Rep. #1 | 267578 | 807102 | 425810 | 20392 | 28693 |
| Rep. #2 | 270766 | 810364 | 429388 | 22250 | 28236 |
| Rep. #3 | 268518 | 805780 | 436152 | 24603 | 28289 |
| Rep. #4 | 339524 | 884027 | 389531 | 16045 | 26290 |
| Rep. #5 | 320596 | 887592 | 389156 | 15241 | 26838 |
| Rep. #6 | 349506 | 885744 | 390760 | 15108 | 26791 |
| Average = | 302748 | 846768 | 410133 | 18940 | 27523 |
| SD = | 38181 | 42785 | 22509 | 4047 | 999 |
| % RSD = | 12.6 | 5.1 | 5.5 | 21.4 | 3.6 |
| 120% | | | | | |
| Rep. #1 | 321374 | 960641 | 516591 | 27030 | 33373 |
| Rep. #2 | 321202 | 968340 | 525659 | 27074 | 33216 |
| Rep. #3 | 319804 | 963105 | 526749 | 27710 | 33820 |
| Rep. #4 | 421097 | 1064456 | 443960 | 19495 | 32209 |
| Rep. #5 | 432519 | 1061758 | 442264 | 18830 | 32265 |
| Rep. #6 | 440981 | 1068369 | 442789 | 18742 | 32963 |
| Average = | 376163 | 1014445 | 483002 | 23147 | 32974 |
| SD = | 60984 | 55324 | 43960 | 4532 | 636 |
| % RSD = | 16.2 | 5.5 | 9.1 | 19.6 | 1.9 |

Standard Linearity

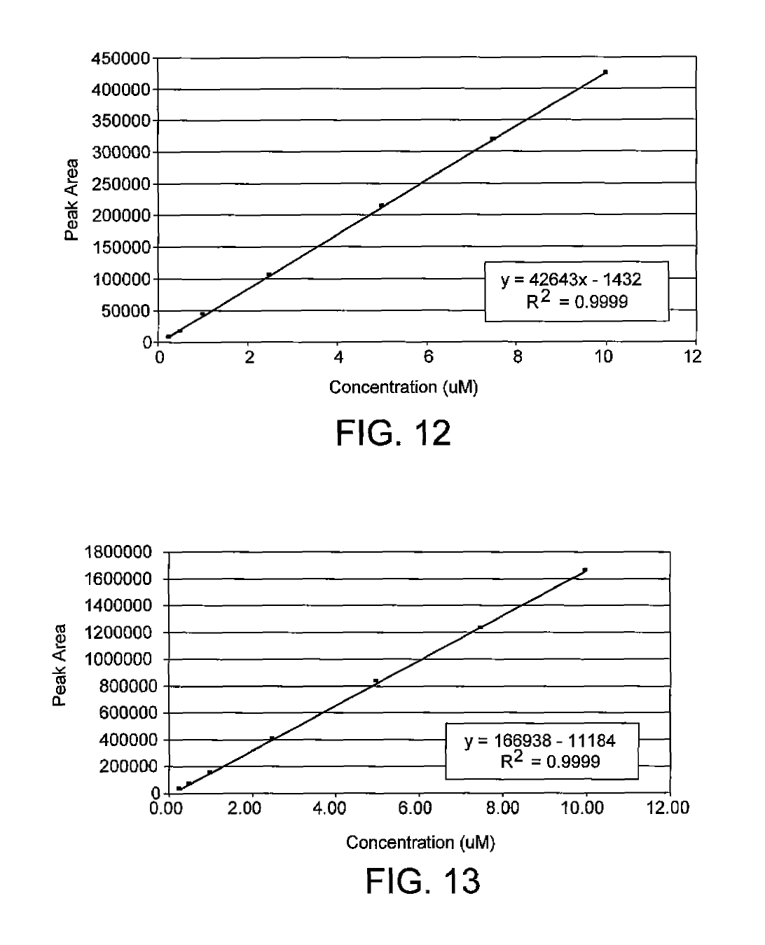

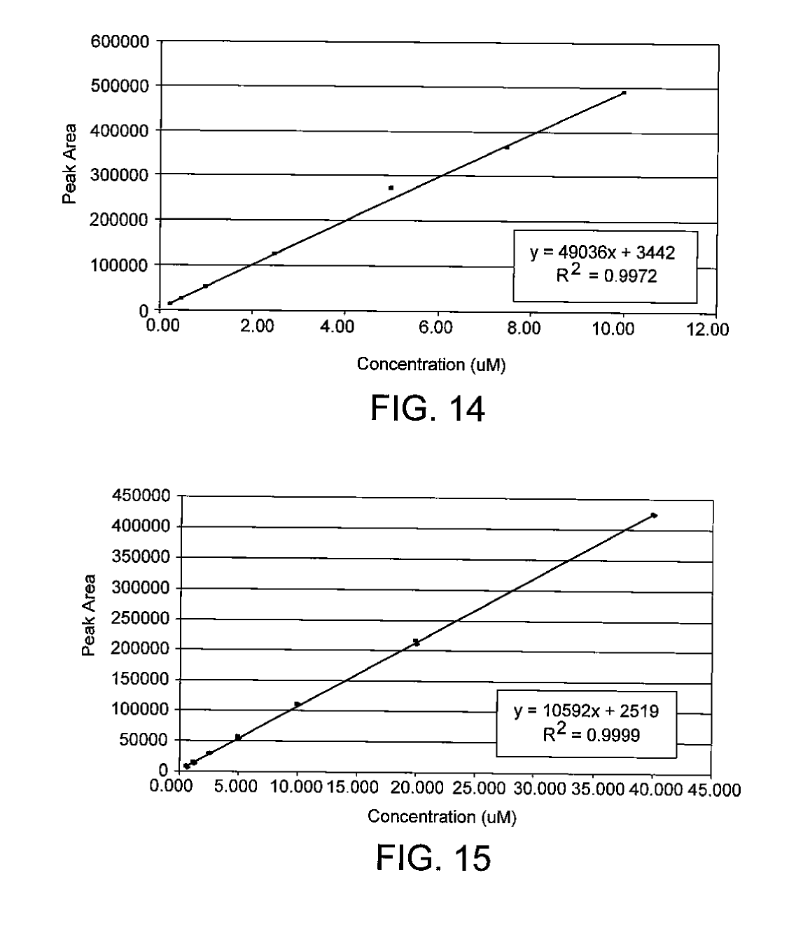

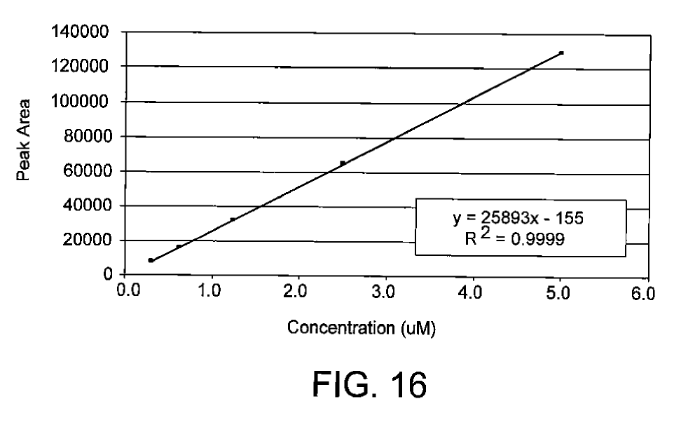

As described above, a series of seven dilutions was evaluated for each impurity. The linear relationships of % nominal amount vs. peak area were calculated using a first-order least squares regression analysis and the data are given in Tables 34 through 38, with Table 39 summarizing the slopes, y-intercepts and correlation coefficients for each of the five compounds. The standard graphs of the data including the correlation coefficient (r²), y-intercept and slope of the regression line are shown in FIGS. 12 through 16 for the DM1 monomer, DM1 dimer, DM1-TPA adduct, PPA and mercaptopyridine. For this study, r² values of 0.9999, 0.9999, 0.9972, 0.9999 and 0.9999 were achieved for the DM1 monomer, DM1 dimer, DM1-TPA adduct, PPA, and mercaptopyridine respectively. Satisfactory linearity was demonstrated for the DM1 monomer, DM1 dimer and DM1-TPA adduct over a range of 10-0.25 μM. For PPA linearity was demonstrated over a range of 40-0.625 μM and for mercaptopyridine linearity was demonstrated over a range of 5-0.312 μM.

TABLE 34
Linearity: Area Response vs. Theoretical Standard Concentration for
DM1 Monomer

| Theoretical | Injection #1 | Injection #2 | Average |
|---|---|---|---|
| concentration (μM) | (area) | (area) | (area) |
| 0.250 | 9019 | 8048 | 8534 |
| 0.500 | 18287 | 16295 | 17291 |
| 1.00 | 43545 | 42839 | 43192 |
| 2.50 | 107069 | 105397 | 106233 |
| 5.00 | 213357 | 212569 | 212963 |
| 7.50 | 317958 | 318674 | 318316 |
| 10.0 | 425959 | 422339 | 424149 |

TABLE 35
Linearity: Area Response vs. Theoretical Standard Concentration for
DM1 Dimer

| Theoretical | | | | |
|---|---|---|---|---|
| concentration | Injection #1 | Injection #2 | Injection #3 | Average |
| (μM) | (area) | (area) | (area) | (area) |
| 0.250 | 35041 | 34092 | 34653 | 34595 |
| 0.500 | 67621 | 69347 | 66904 | 67957 |
| 1.00 | 151815 | 152428 | 154590 | 152944 |
| 2.50 | 408926 | 406022 | 407410 | 407453 |
| 5.00 | 834627 | 830127 | 828352 | 831035 |
| 7.50 | 1229558 | 1235151 | 1233380 | 1232696 |
| 10.0 | 1659946 | 1658940 | 1662968 | 1660618 |

TABLE 36
Linearity: Area Response vs. Theoretical Standard Concentration for
DM1 TPA

| Theoretical | Injection #1 | Injection #2 | Average |
|---|---|---|---|
| concentration (μM) | (area) | (area) | (area) |
| 0.250 | 12789 | 14159 | 13474 |
| 0.500 | 24308 | 24265 | 24287 |
| 1.00 | 50901 | 51102 | 51002 |
| 2.50 | 123922 | 123727 | 123825 |
| 5.00 | 298396 | 242328 | 270362 |
| 7.50 | 363774 | 360835 | 362305 |
| 10.0 | 490215 | 490881 | 490548 |

TABLE 37
Linearity: Area Response vs. Theoretical Standard Concentration for
PPA

| Theoretical | | | | |
|---|---|---|---|---|
| concentration | Injection #1 | Injection #2 | Injection #3 | Average |
| (μM) | (area) | (area) | (area) | (area) |
| 0.6 | 7086 | 7530 | 7549 | 7388 |
| 1.3 | 14576 | 14507 | 14693 | 14592 |
| 2.5 | 28540 | 28469 | 28519 | 28509 |
| 5.0 | 56619 | 56731 | 55518 | 56289 |
| 10 | 110121 | 110817 | 110624 | 110521 |
| 20 | 220163 | 214111 | 211527 | 215267 |
| 40 | 424037 | 425898 | 425431 | 425122 |

TABLE 38
Linearity: Area Response vs. Theoretical Standard Concentration for
Mercaptopyridine

| Theoretical | | | | |
|---|---|---|---|---|
| concentration | Injection #1 | Injection #2 | Injection #3 | Average |
| (μM) | (area) | (area) | (area) | (area) |
| 0.08 | 1719 | 1707 | 1502 | 1657 |
| 0.2 | 3668 | 3783 | 3511 | 3654 |
| 0.3 | 7679 | 7733 | 7733 | 7715 |
| 0.6 | 15919 | 15908 | 15908 | 15912 |
| 1.3 | 32281 | 32363 | 31802 | 32149 |
| 2.5 | 64696 | 65217 | 65688 | 65267 |
| 5.0 | 129304 | 128858 | 128861 | 129008 |

TABLE 39
Linear Regression Analysis of DM1 Monomer, DM1 Dimer, DM1-
TPA, PPA and Mercaptopyridine Standard Curves

| | | | Correlation | Standard |
|---|---|---|---|---|
| Compound | Slope | y-intercept | coefficient (r²) | error_{y intercept} |
| DM1 monomer | 42643 | −1432 | 0.9999 | 1047 |
| DM1 dimer | 166938 | −11184 | 0.9999 | 3290 |
| DM1-TPA adduct | 49036 | 3442 | 0.9972 | 6053 |
| PPA | 10592 | 2519 | 0.9999 | 699 |
| Mercaptopyridine | 25893 | −155 | 0.9999 | 182 |

Spike Recovery (Accuracy) and Linearity

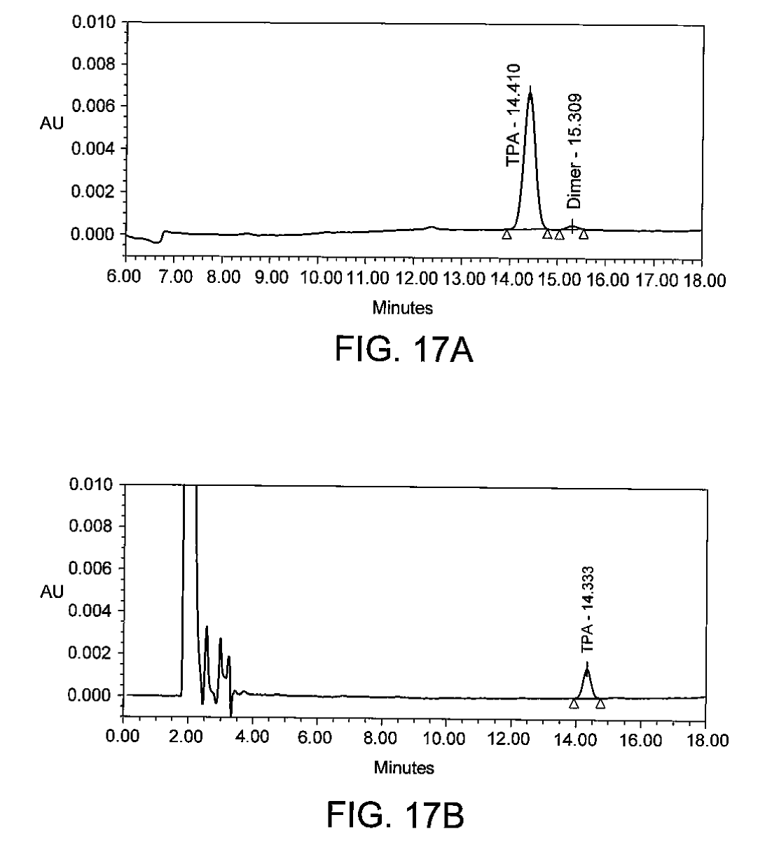

FIG. 17 displays representative chromatograms of unspiked DS-DM1-deJ591. The injection volume was 50 μl and peak detection was at 252 nm (where DM1-TPA and DM1-dimer peaks were detected, and the DM1-monomer peak was absent) and 280 nm (where PPA and mercaptopyridine peaks were not detected).

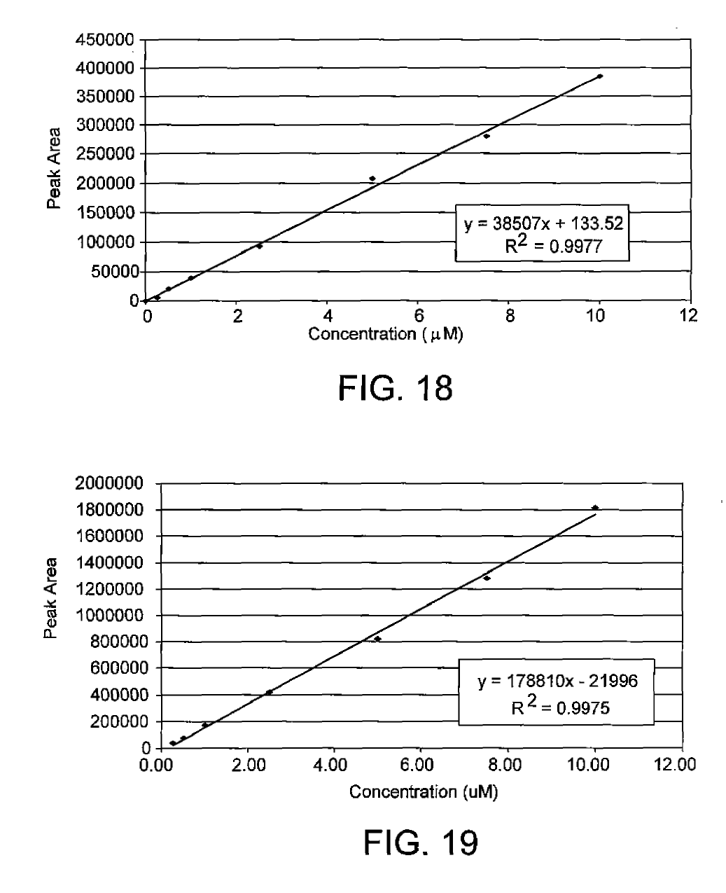

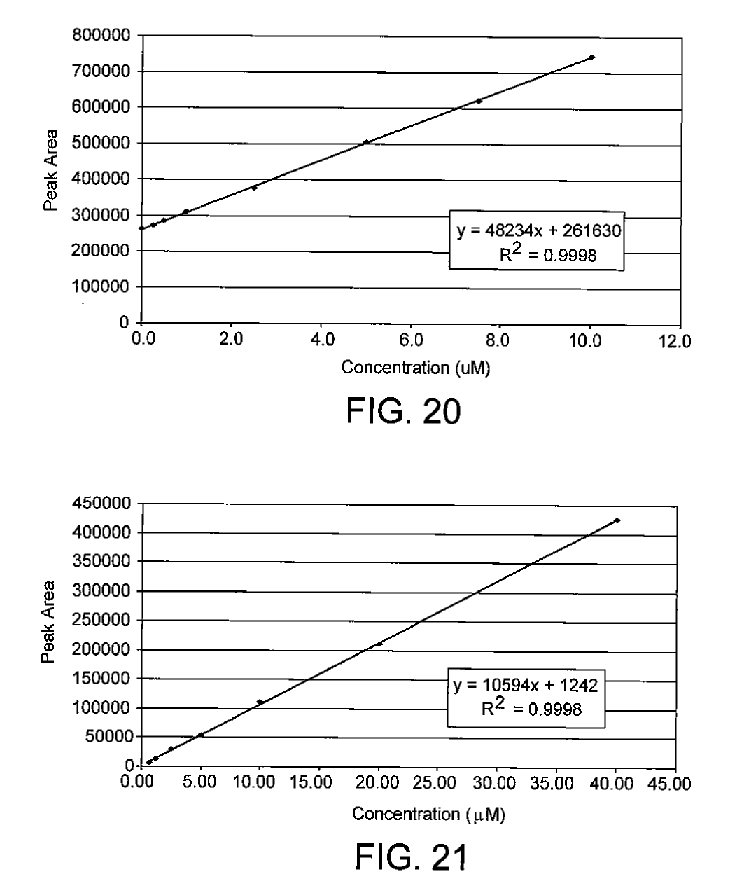

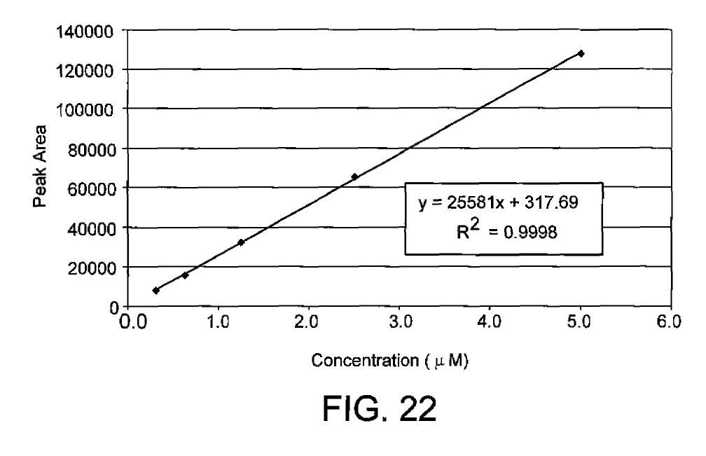

The linear relationships of % nominal amount vs. peak area were calculated for the DM1-related species in the presence of DS-DM1-deJ591 using a first-order least squares regression analysis and the data are given in Tables 40 through 44, with Table 45 summarizing the slopes, y-intercepts and correlation coefficients for each of the five compounds. The standard graphs of the data including the correlation coefficient (r²), y-intercept and slope of the regression line are shown in FIGS. 18 through 22 for the DM1 monomer, DM1 dimer and DM1-TPA, PPA, and mercaptopyridine, respectively. For this study, r² values of 0.9977, 0.9975, 0.9998, 0.9998 and 0.9998 were achieved for the DM1 monomer, DM1 dimer, DM1-TPA, PPA and mercaptopyridine respectively. Satisfactory linearity was demonstrated for the DM1 monomer, DM1 dimer, and DM1-TPA in the presence of DS-DM1-deJ591 over a range of 0.25-10 μM. Satisfactory linearity was demonstrated for PPA and mercaptopyridine in the presence of DS-DM1-deJ591 over the ranges of 0.625-40 μM and 0.312-5 μM, respectively.

TABLE 40
Spike Recovery: Area Response vs. Theoretical Concentration for
DM1 Monomer Spiked into DS-DM1-deJ591

| Theoretical | Injection #1 | Injection #2 | Average |
|---|---|---|---|
| concentration (μM) | (area) | (area) | (area) |
| 0.000 | 0 | 0 | 0 |
| 0.250 | 5449 | 5399 | 5424 |
| 0.500 | 21713 | 19076 | 20395 |
| 1.00 | 39921 | 38483 | 39202 |
| 2.50 | 91517 | 94279 | 92898 |
| 5.00 | 207750 | 208038 | 207894 |
| 7.50 | 282899 | 278254 | 280577 |
| 10.0 | 386846 | 382614 | 384730 |

TABLE 41
Spike Recovery - Area Response vs. Theoretical Concentration for
DM1 Dimer Spiked into DS-DM1-deJ591

| Theoretical | | | | |
|---|---|---|---|---|
| concentration | Injection #1 | Injection #2 | Injection #3 | Average |
| (μM) | (area) | (area) | (area) | (area) |
| 0.250 | 37356 | 38056 | 36552 | 37321 |
| 0.500 | 77245 | 74643 | 76389 | 76092 |
| 1.00 | 173507 | 173623 | 173893 | 173674 |
| 2.50 | 420454 | 419662 | 421289 | 420468 |
| 5.00 | 821521 | 821319 | 821237 | 821359 |
| 7.50 | 1282041 | 1284305 | 1284027 | 1283458 |
| 10.0 | 1823007 | 1816961 | 1810502 | 1816823 |

TABLE 42
Spike Recovery - Area Response vs. Theoretical Concentration for
DM1-TPA Adduct Spiked into DS-DM1-deJ591

| Theoretical | Injection #1 | Injection #2 | Average |
|---|---|---|---|
| concentration (μM) | (area) | (area) | (area) |
| 0.000 | 265537 | 261834 | 263686 |
| 0.250 | 275365 | 271237 | 273301 |
| 0.500 | 286799 | 285578 | 286189 |
| 1.00 | 313207 | 309303 | 311255 |
| 2.50 | 378801 | 376921 | 377861 |
| 5.00 | 504803 | 503450 | 504127 |
| 7.50 | 624074 | 616740 | 620407 |
| 10.0 | 748542 | 744422 | 746482 |

TABLE 43
Spike Recovery: Area Response vs. Theoretical Concentration for
PPA Spiked into DS-DM1-deJ591

| Theoretical | Injection #1 | Injection #2 | Injection #3 | Average |
|---|---|---|---|---|
| concentration (μM) | (area) | (area) | (area) | (area) |
| 0.625 | 6326 | 6251 | 6198 | 6258 |
| 1.25 | 12916 | 12771 | 13184 | 12957 |
| 2.5 | 29383 | 28856 | 28931 | 29057 |
| 5 | 54400 | 54241 | 53896 | 54179 |
| 10 | 110526 | 111258 | 111570 | 111118 |
| 20 | 210810 | 211120 | 210775 | 210902 |
| 40 | 424037 | 425898 | 425431 | 425122 |

TABLE 44
Spike Recovery - Area Response vs. Theoretical Concentration for
Mercaptopyridine Spiked into DS-DM1-deJ591

| Theoretical | Injection #1 | Injection #2 | Injection #3 | Average |
|---|---|---|---|---|
| concentration (μM) | (area) | (area) | (area) | (area) |
| 0.312 | 7763 | 7487 | 7639 | 7630 |
| 0.625 | 16144 | 16226 | 16077 | 16149 |
| 1.25 | 32728 | 32545 | 33025 | 32766 |
| 2.5 | 64794 | 64919 | 65570 | 65094 |
| 5.0 | 127511 | 127479 | 128281 | 127757 |

TABLE 45
Linear Regression Analysis of DM1 Monomer, DM1 Dimer, DM1-
TPA Adduct, PPA, and Mercaptopyridine Spike Recovery Curves

| | | | Correlation | Standard |
|---|---|---|---|---|
| Compound | Slope | y-intercept | coefficient (r²) | error_{y intercept} |
| DM1 monomer | 38507 | 134 | 0.9977 | 4564 |
| DM1 dimer | 178810 | −21996 | 0.9975 | 20891 |
| DM1-TPA adduct | 48234 | 261630 | 0.9998 | 1515 |
| PPA | 10594 | 1242 | 0.9998 | 854 |
| Mercaptopyridine | 25581 | 318 | 0.9998 | 316 |

The accuracy of the method in the presence of DS-DM1-deJ591 was determined at concentration levels of DM1 monomer, DM1 dimer, and DM1-TPA ranging from 0.25-10 μM. For PPA the concentration range was from 0.625-40 μM and for mercaptopyridine the concentration range was from 0.155-5.0 μM. Unspiked samples were also prepared and used in the determination of accuracy. As described above, the DM1 dimer study was performed by a second analyst, using triplicate samples at three concentrations bracketing the working concentration. The percent recoveries of the spiked samples are shown in Tables 46 through 51. The average % recovery from the spiked samples in 50% acetonitrile/0.01% TFA were determined to be 88.80%, 100.05%, 87.65%, 92.74%, and 99.32% for the DM1 monomer, DM1 dimer, DM1-TPA, PPA and mercaptopyridine, respectively. For the DM1 dimer study performed by the second analyst, recovery was determined to be 96.03%. The ranges of percent recoveries observed were: 58.77-102.54% for the DM1 monomer, 92.04-108.91 for the DM1 dimer, 61.24-97.77% for the DM1-TPA adduct, 68.48-102.47% for PPA and 96.29-101.72% for mercaptopyridine.

Significantly low recoveries were obtained at the lowest concentration examined for the DM1 monomer, DM1-TPA, and PPA. By excluding the lowest spike concentration and assessing accuracy for the six remaining spiking concentration levels of these DM1-related impurities (i.e., from 0.50-10 μM for the DM1 monomer and DM1-TPA, and 1.25-40 μM for PPA), the average % recovery from the spiked samples were determined to be 93.80%, 92.05% and 96.78% for the DM1 monomer, DM1-TPA, and PPA. The ranges of % recoveries over this modified concentration range were: 88.12-102.54% for the DM1 monomer, 91.22-98.55% for the DM1-TPA adduct and 80.48-97.77% for PPA.

Thus, the method demonstrates good accuracy for the determination of DM1 monomer, DM1 dimer, DM1-TPA, PPA, and mercaptopyridine in the presence of DS-DM1-deJ591.

TABLE 46
Spike Recovery/Accuracy of DM1 Monomer in DS-DM1-deJ591

| | | Corrected | | | |
|---|---|---|---|---|---|
| DM1 monomer | Average | average | Theo- | | |
| concentration | experimental | experimental | retical | % | |
| (μM) | area | area^{a} | area^{b} | recovery | |
| 0.250 | 5424 | 5424 | 9229 | 58.77 | |
| 0.500 | 20395 | 20395 | 19890 | 102.54 | |
| 1.00 | 39202 | 39202 | 41211 | 95.12 | |
| 2.50 | 92898 | 92898 | 105176 | 88.33 | |
| 5.00 | 207894 | 207894 | 211783 | 98.16 | |
| 7.50 | 280577 | 280577 | 318391 | 88.12 | |
| 10.0 | 384730 | 384730 | 424998 | 90.53 | |
| | | | average | 88.80 | 93.8^{c} |
| | | | SD | 14.27 | 5.83^{c} |
| | | | % RSD | 16.07 | 6.22^{c} |

^{a}Corrected experimental areas were generated by subtracting the average integrated area from DM1 monomer present in unspiked DS-DM1-deJ591.
^{b}Theoretical areas were generated using the best fit line from the linear regression analysis of the DM1 monomer linearity standards and the DM1 monomer concentration from the spike.
^{c}Average and standard deviation calculated following removal of lowest concentration spike from data set.

TABLE 47
Spike Recovery/Accuracy of DM1 Dimer in DS-DM1-deJ591 in 50%
Acetonitrile/0.01% TFA

| DM1 dimer | | Corrected | | |
|---|---|---|---|---|
| concentration | Experimental | experimental | Theoretical | % |
| (μM) | area | area^{a} | area^{b} | Recovery |
| 0.250 | 37321 | 37321 | 40550 | 92.04 |
| 0.500 | 76092 | 76092 | 82284 | 92.47 |
| 1.00 | 173674 | 173674 | 165753 | 104.78 |
| 2.50 | 420468 | 420468 | 416160 | 101.04 |
| 5.00 | 821359 | 821359 | 833505 | 98.54 |
| 7.50 | 1283458 | 1283458 | 1250850 | 102.61 |
| 10.0 | 1816823 | 1816823 | 1668195 | 108.91 |
| | | | Average | 100.05 |
| | | | SD | 6.22 |
| | | | % RSD | 6.22 |

^{a}Corrected experimental areas were generated by subtracting the average integrated area from DM1 dimer present in unspiked DS-DM1-deJ591.
^{b}Theoretical areas were generated using the best fit line from the linear regression analysis of the DM1 dimer linearity standards and the DM1 dimer concentration from the spike.

TABLE 48
Spike Recovery/Accuracy of DM1 Dimer in DS-DM1-deJ591 in 50%
Acetonitrile/0.01% TFA (Second Analyst)

| DM1 dimer | Spiked area | Unspiked area | % |
|---|---|---|---|
| concentration (μM) | (n = 3) | (n = 3) | Recovery |
| 4 | 599385 | 607347 | 98.69 |
| 5 | 755096 | 807749 | 93.48 |
| 6 | 924581 | 964029 | 95.91 |
| | | Average | 96.03 |
| | | SD | 2.61 |
| | | % RSD | 2.71 |

TABLE 49
Spike Recovery/Accuracy of DM1-TPA Adduct in DS-DM1-deJ591

| DM1-TPA | | | | | |
|---|---|---|---|---|---|
| adduct | Ex- | Corrected | Theo- | | |
| concentration | perimental | experimental | retical | % | |
| (μM) | area | area^{a} | area^{b} | Recovery | |
| 0.250 | 273301 | 9616 | 15701 | 61.24 | |
| 0.500 | 286189 | 22503 | 27960 | 80.48 | |
| 1.00 | 311255 | 47570 | 52478 | 90.65 | |
| 2.50 | 377861 | 114176 | 126032 | 90.59 | |
| 5.00 | 504127 | 240441 | 248622 | 96.71 | |
| 7.50 | 620407 | 356722 | 371212 | 96.10 | |
| 10.0 | 746482 | 482797 | 493802 | 97.77 | |
| | | | Average | 87.65 | 92.05^{c} |
| | | | SD | 13.05 | 6.46^{c} |
| | | | % RSD | 14.89 | 7.02^{c} |

^{a}Corrected experimental areas were generated by subtracting the average integrated area from DM1-TPA adduct present in unspiked DS-DM1-deJ591.
^{b}Theoretical areas were generated using the best fit line from the linear regression analysis of the DM1-TPA adduct linearity standards and the DM1-TPA adduct concentration from the spike.
^{c}Average and standard deviation calculated following removal of lowest concentration spike from data set

TABLE 50
Spike Recovery/Accuracy of PPA Adduct in DS-DM1-deJ591

| PPA adduct | Experi- | Corrected | Theo- | | |
|---|---|---|---|---|---|
| concentration | mental | experimental | retical | % | |
| (μM) | area | area^{a} | area^{b} | Recovery | |
| 0.625 | 6258 | 6258 | 9139 | 68.48 | |
| 1.25 | 12957 | 12957 | 15759 | 82.22 | |
| 2.5 | 29057 | 29057 | 28999 | 100.2 | |
| 5 | 54179 | 54179 | 55479 | 97.66 | |
| 10 | 111118 | 111118 | 108439 | 102.47 | |
| 20 | 210902 | 210902 | 214359 | 98.39 | |
| 40 | 425122 | 425122 | 426199 | 99.75 | |
| | | | Average | 92.74 | 96.78^{c} |
| | | | SD | 12.61 | 7.32^{c} |
| | | | % RSD | 13.60 | 7.57^{c} |

^{a}Corrected experimental areas were generated by subtracting the average integrated area from PPA adduct present in unspiked DS-DM1-deJ591.
^{b}Theoretical areas were generated using the best fit line from the linear regression analysis of the PPA adduct linearity standards and the PPA adduct concentration from the spike.
^{c}Average and standard deviation calculated following removal of lowest concentration spike from data set.

TABLE 51
Spike Recovery/Accuracy of Mercaptopyridine in DS-DM1-deJ591

| Mercaptopyridine | | Corrected | | |
|---|---|---|---|---|
| concentration | Experimental | experimental | Theoretical | % |
| (μM) | area | area^{a} | area^{b} | Recovery |
| 0.155 | 3764 | 3764 | 3764 | 97.55 |
| 0.312 | 7630 | 7630 | 7924 | 96.29 |
| 0.625 | 16149 | 16149 | 16028 | 100.75 |
| 1.25 | 32766 | 32766 | 32214 | 101.72 |
| 2.5 | 65094 | 65094 | 64578 | 100.80 |
| 5.0 | 127757 | 127757 | 129310 | 98.80 |
| | | | Average | 99.32 |
| | | | SD | 2.13 |
| | | | % RSD | 2.14 |

^{a}Corrected experimental areas were generated by subtracting the average integrated area from mercaptopyridine present in unspiked DS-DM1-deJ591.
^{b}Theoretical areas were generated using the best fit line from the linear regression analysis of the mercaptopyridine linearity standards and the mercaptopyridine concentration from the spike.

Limit of Detection (LOD) and Limit of Quantitation (LOQ)

Triplicates of the DM1-related impurities standard solutions over a range of 0.075-10 μM were used to determine the LOD for DM1 monomer, DM1 dimer and DM1-TPA. Ranges of 0.077-40 μM and from 0.039-5 μM were used for PPA and mercaptopyridine, respectively. The LOD for each of the impurities is summarized in Table 52. The LOD's established were 0.025 μM for DM1 monomer, 0.125 μM for DM1 dimer, 0.015 μM for DM1-TPA, 0.155 μM for PPA, and 0.075 μM for mercaptopyridine.

Triplicates used for the LOD estimation were also used for the calculation of LOQ. The LOQ for each of the impurities is summarized in Table 52. The LOQ's were established to be 0.125 μM for DM1 monomer, 0.25 μM for DM1 dimer, 0.062 μM for DM1-TPA, 0.312 μM for PPA, and 0.15 μM for mercaptopyridine.

TABLE 52
Estimates of LOD and LOQ for DM1 Monomer, DM1 Dimer,
DM1-TPA Adduct, PPA, and Mercaptopyridine in Standard Solutions

| | LOD - standard | LOQ - standard |
|---|---|---|
| Compound | solution (μM) | solution (μM) |
| DM1 monomer | 0.025 | 0.125 |
| DM1 dimer | 0.125 | 0.25 |
| DM1-TPA adduct | 0.015 | 0.062 |
| PPA | 0.155 | 0.312 |
| Mercaptopyridine | 0.075 | 0.15 |

Range

The range of the method was determined from the data obtained from the linearity studies. A range of 10-0.5 μM for the DM1 monomer, 10-0.25 μM for DM1 dimer, 10-0.5 μM for DM1-TPA, 40-1.25 μM for PPA, and of 5-0.155 μM for mercaptopyridine was established.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of quantifying unconjugated anti-PSMA antibody molecules in a sample comprising maytansinoid conjugated anti-PSMA antibody molecules, the method comprising:
(1) contacting the sample with a maytansinoid-specific antibody to deplete maytansinoid conjugated antibody molecules from the sample and to form a depleted sample;
(2) contacting at least a portion of the depleted sample with an anti-PSMA antibody-specific binding agent; and
(3) evaluating the presence or amount of anti-PSMA antibody bound by the anti-PSMA antibody-specific binding agent.

2. The method of claim 1, wherein the evaluation includes comparing the fraction or amount of unconjugated antibody molecules in the depleted sample with a reference value.

3. The method of claim 1, wherein the sample comprises a biological fluid.

4. The method claim 1, wherein the sample is from a batch of conjugated antibody molecules.

5. The method of claim 1, wherein the sample is a formulated conjugated antibody product.

6. The method of claim 1, wherein the anti-PSMA antibody molecule is selected from the group consisting of E99, J415, J533, J591, deJ591 and antigen-binding fragments thereof.

7. The method of claim 1, wherein the conjugate comprises a maytansinol.

8. The method of claim 1, wherein the conjugate molecule comprises DM1.

9. The method of claim 1, wherein the antibody-specific antibody is directly or indirectly labeled.

10. The method claim 1, wherein the sample comprises DS-DM1-deJ591.

11. The method of claim 1, wherein the sample comprises a formulated DS-DM1-deJ591 product.

12. A method of evaluating the stability of a sample comprising a maytansinoid-conjugated anti-PSMA antibody molecule, the method comprising:
providing a first portion of the sample at a first period of time;
depleting maytansinoid-conjugated anti-PSMA antibody molecules from the first portion, using an antibody molecule that specifically binds the maytansinoid;
detecting anti-PSMA antibody molecules remaining in the first portion, thereby determining a first level of anti-PSMA unconjugated antibody molecules in the sample;
providing a second portion of the sample at a second period of time;
depleting substantially all of the maytansinoid-conjugated anti-PSMA antibody molecules from the second portion using an antibody molecule that specifically binds the maytansinoid; and
detecting anti-PSMA antibody molecules remaining in the second aliquot, thereby determining a second level of unconjugated antibody molecules,
wherein a change in the level of unconjugated anti-PSMA antibody molecules between the first portion and the second portion is indicative of the stability of the maytansinoid-conjugated anti-PSMA antibody molecule in the sample.

13. A method of providing or calculating a dosage of a conjugated antibody comprising:
providing an evaluation of the amount of degradation of a conjugated antibody which has occurred or will occur in a test sample wherein the evaluation is provided by the method of claim 1 or 12;
comparing the amount of degradation to a reference value, and providing or calculating a dosage based on the relationship of the sample value to the reference value.

14. The method of claim 13, wherein the sample value is greater than the reference value and the dosage is increased.

15. A method of evaluating the level of free or unconjugated DM1 in a sample, comprising: contacting the sample with a moiety which reacts with the free or unconjugated DM1 to form derivatized DM1, which is preferably more stable than free or unconjugated DM1, and detecting the derivatized DM1 in the sample.

16. The method of claim 15, wherein the moiety is pyridyl disulfide.

17. A method of analyzing a sample containing a mixture of anti-PSMA antibody molecules conjugated to DM1 or DOTA comprising:
using MALDI-TOF MS to resolve and identify the different masses of antibody isoforms representing various levels of DM1 or DOTA conjugation.

18. A method of analyzing a sample containing a mixture of anti-PSMA antibody molecules conjugated to DM1 or DOTA comprising:
using MALDI-TOF MS to quantitate and determine concentration level for at least one, two or three detected antibody isoform.

19. The method of claim 18, comprising using MALDI-TOF MS to quantitate and determine concentration level for each detected antibody isoform

20. The method of claim 17, wherein, distribution ratios for conjugation of either DOTA or DM1 to antibody are determined by selecting a peak and analyzing it by Gaussian deconvolution and peak fitting.

21. A method of quantifying one or more DM1-related impurities in a sample comprising DM1-conjugated antibody molecules, the method comprising:
(1) applying a sample to a chromatography matrix capable of separating at least one of DM1-related impurities selected from: DM1 monomer, DM1 dimer, DM1-TPA adduct, 4-(2-pyridyldithio) pentanoic acid (PPA) and mercaptopyridine from the sample; and
(2) evaluating the presence or amount of one or more DM1-related impurities, thereby quantifying one or more DM1-related impurities in the sample.

22. The method of claim 21, wherein the evaluating step comprises comparing the fraction or amount of one or more DM1-related impurities in the sample with a reference value.

23. The method of claim 21, wherein the sample comprises a biological fluid.

24. The method claim 21, wherein the sample is from a batch of DM1-conjugated antibody molecules.

25. The method of claim 21, wherein the sample is a formulated DM1-conjugated antibody product.

26. The method of claim 21, wherein the DM1-conjugated antibody molecule comprises an anti-PSMA antibody molecule.

27. The method claim 21, wherein the sample comprises DS-DM1-deJ591.

28. The method of claim 21, wherein the sample comprises a formulated DS-DM1-deJ591 product.

29. The method of claim 21, wherein the separating step comprises chromatography.

30. The method of claim 21, wherein the chromatography is high performance liquid chromatography (HPLC).
